(19) United States
(12) Patent Application Publication
(10) Pub. No.: US 2026/0324279 A1
(43) Pub. Date: Oct. 15, 2026

(54) LIGHT-EMITTING DEVICE AND ELECTRONIC APPARATUS INCLUDING THE SAME

(71) Applicant: Samsung Display Co., Ltd., Yongin-si (KR)

(72) Inventors: Gyeongheon Kim, Yongin-si (KR); Hoilim Kim, Yongin-si (KR)

(21) Appl. No.: 19/326,971

(22) Filed: Sep. 12, 2025

(30) Foreign Application Priority Data
Apr. 2, 2025 (KR) 10-2025-0043049

(51) Int. Cl. H10K 85/60; C07C 255/31; C07C 255/52; C07D 471/14; C09K 11/06; H10K 50/12; H10K 50/15; H10K 50/17; H10K 85/30

(52) U.S. Cl. CPC H10K 85/623; C07C 255/31; C07C 255/52; C07D 471/14; C09K 11/06; H10K 85/346; H10K 85/625; C09K 2211/1007; C09K 2211/1014; C09K 2211/1022; C09K 2211/1029; C09K 2211/1044; C09K 2211/185; H10K 50/12; H10K 50/15; H10K 50/17

(57) ABSTRACT

Provided is a light-emitting device including a compound represented by Formula 1, a first electrode, a second electrode facing the first electrode, and an interlayer arranged between the first electrode and the second electrode and including an emission layer, wherein the interlayer includes at least one compound represented by Formula 1:

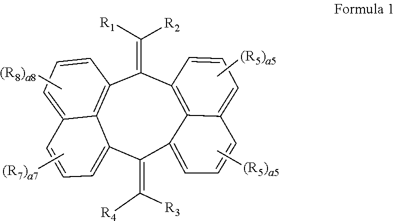

wherein Formula 1 comprises an electron-withdrawing group (EWG), and the remaining substituents of Formula 1 are as provided herein.

### CROSS-REFERENCE TO RELATED APPLICATION

This application claims priority to and the benefit of Korean Patent Application No. 10-2025-0043049, filed on Apr. 2, 2025, in the Korean Intellectual Property Office, and all the benefits accruing therefrom under 35 U.S.C. § 119, the content of which is incorporated by reference herein in its entirety.

### BACKGROUND

### 1. Field

One or more embodiments relate to a light-emitting device and an electronic apparatus or the like including the same.

### 2. Description of the Related Art

From among light-emitting devices, self-emissive devices have wide viewing angles, high contrast ratios, short response times, and excellent characteristics in terms of luminance, driving voltage, and response speed.

In a light-emitting device, a first electrode is arranged on a substrate, and a hole transport region, an emission layer, an electron transport region, and a second electrode are sequentially arranged on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. The excitons may transition from an excited state to a ground state, thereby generating light.

### SUMMARY

One or more embodiments include an excellent light-emitting device or the like.

Additional aspects will be set forth in part in the detailed description that follows and, in part, will be apparent from the detailed description, or may be learned by practice of the presented exemplary embodiments.

According to an aspect, a light-emitting device includes:
a first electrode,
a second electrode facing the first electrode, and
an interlayer arranged between the first electrode and the second electrode, wherein the interlayer includes an emission layer,
wherein the interlayer further includes at least one compound represented by Formula 1:

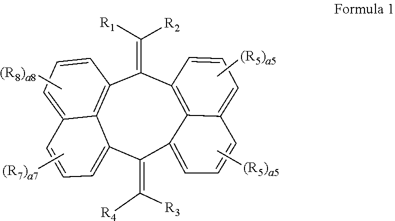

wherein, in Formula 1,
R₁ to R₈ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryl group unsubstituted or substituted with at least one R₁₀ₐ, a C₇-C₆₀ aryl alkyl group unsubstituted or substituted with at least one R₁₀ₐ, a C₇-C₆₀ alkyl aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₂-C₆₀ alkyl heteroaryl group unsubstituted or substituted with at least one R₁₀ₐ, a C₂-C₆₀ heteroaryl alkyl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a C₈-C₆ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)(Q₁), —S(═O)₂(Q₁), —P(Q₁)(Q₂), or —P(═O)(Q₁)(Q₂),
a5 to a8 are each independently an integer from 1 to 3, wherein, when a5 is 2 or more, each R₅ is identical to or different from the others, when a6 is 2 or more, each R₆ is identical to or different from the others, when a7 is 2 or more, each R₇ is identical to or different from the others, and when a8 is 2 or more, each R₈ is identical to or different from the others,
R₁₀ₐ is:
hydrogen, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, or a phosphoric acid group or a salt thereof,
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O)(Q₁₁), —S(═O)(Q₁₁), —S(═O)₂(Q₁₁), —P(Q₁₁)(Q₁₂), —P(═O)(Q₁₁)(Q₁₂), or a combination thereof,
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, or a C₁-C₆₀ heteroarylthio group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, or a C₁-C₆₀ heteroarylthio group, —Si(Q₂₁)(Q₂₂)(Q₂₃), —N(Q₂₁)(Q₂₂), —B(Q₂₁)(Q₂₂), —C(═O)(Q₂₁), —S(═O)(Q₂₁), —S(═O)₂(Q₂₁), —P(Q₂₁)(Q₂₂), —P(═O)(Q₂₁)(Q₂₂), or a combination thereof, or

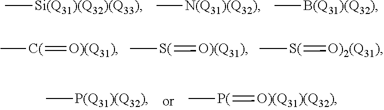

Q₁ to Q₃, Q₁₁ to Q₁₃, Q₂₁ to Q₂₃, and Q₃₁ to Q₃₃ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, or
a C₃-C₆₀ carbocyclic group, a C₁-C₆₀ heterocyclic group, a C₇-C₆₀ aryl alkyl group, a C₂-C₆₀ heteroaryl alkyl group, a C₆-C₆₀ aryloxy group, a C₆-C₆₀ arylthio group, a C₁-C₆₀ heteroaryloxy group, or a C₁-C₆₀ heteroarylthio group,
each unsubstituted or substituted with deuterium, —F, a cyano group, a C₁-C₆₀ alkyl group, a C₁-C₆₀ alkoxy group, a C₁-C₆₀ alkylthio group, a phenyl group, a biphenyl group, or a combination thereof, and
wherein Formula 1 includes an electron-withdrawing group (EWG).

According to an aspect, an electronic apparatus includes the light-emitting device.

According to an aspect, provided is the compound represented by Formula 1.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1 and 2 are each a schematic view of a structure of a light-emitting device according to one or more embodiments;

FIG. 3 is a cross-sectional view of an electronic apparatus according to one or more embodiments;

FIG. 4 is a cross-sectional view of an electronic apparatus according to another embodiment;

FIG. 5 is a block diagram of an electronic apparatus according to one or more embodiments; and

FIG. 6 is a schematic view of an electronic apparatus according to various embodiments.

### DETAILED DESCRIPTION

Reference will now be made in further detail to exemplary embodiments, examples of which are illustrated in the accompanying drawings, wherein like reference numerals refer to like elements throughout. In this regard, the present embodiments may have different forms and should not be construed as being limited to the detailed descriptions set forth herein. Accordingly, the embodiments are merely described below, and by referring to the figures, to explain certain aspects and features of the present detailed description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

The terminology used herein is for the purpose of describing one or more exemplary embodiments only and is not intended to be limiting. As used herein, the singular forms “a,” “an,” and “the” are intended to include the plural forms as well, unless the context clearly indicates otherwise. The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

It will be understood that when an element is referred to as being “on” another element, it can be directly in contact with the other element or intervening elements may be present therebetween. In contrast, when an element is referred to as being “directly on” another element, there are no intervening elements present.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

Efficiency, driving, and lifespan characteristics of light-emitting devices need to be continuously improved, and to this end, materials for forming a hole injection layer used in a hole transport region are under constant development. In this regard, a p-dopant material used in a hole injection layer is developed to improve performance of light-emitting devices.

An aspect provides a light-emitting device including:
a first electrode;
a second electrode facing the first electrode; and
an interlayer arranged between the first electrode and the second electrode, wherein the interlayer includes an emission layer, and
wherein the interlayer includes at least one compound represented by Formula 1:

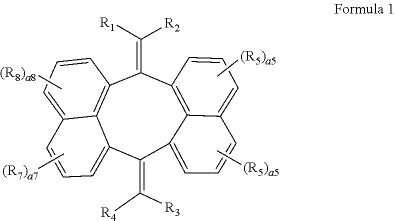

wherein, in Formula 1,
R₁ to R₈ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryl group unsubstituted or substituted with at least one R₁₀ₐ, a C₇-C₆₀ alkyl aryl group unsubstituted or substituted with at least one R₁₀ₐ, a C₇-C₆₀ aryl alkyl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₂-C₆₀ alkyl heteroaryl group unsubstituted or substituted with at least one R₁₀ₐ, a C₂-C₆₀ heteroaryl alkyl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a C₈-C₆ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)(Q₁), —S(═O)₂(Q₁), —P(Q₁)(Q₂), or —P(═O)(Q₁)(Q₂),
a5 to a8 are each independently an integer from 1 to 3, wherein, when a5 is 2 or more, each R₅ is identical to or different from the others, when a6 is 2 or more, each R₆ is identical to or different from the others, when a7 is 2 or more, each R₇ is identical to or different from the others, and when a8 is 2 or more, each R₈ is identical to or different from the others,
R₁₀ₐ is:
hydrogen, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, or a phosphoric acid group or a salt thereof;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)(Q₁₁), —S(═O)₂(Q₁₁), —P(Q₁₁)(Q₁₂), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ aryl alkyl group, or a C₂-C₆₀ heteroaryl alkyl group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)(Q₂₁), —S(═O)₂(Q₂₁), —P(Q₂₁)(Q₂₂), —P(═O)(Q₂₁)(Q₂₂), or a combination thereof; or
Si(Q₃₁)(Q₃₂)(Q₃₃), —N(Q₃₁)(Q₃₂), —B(Q₃₁)(032), —C(═O)(Q₃₁), —S(═O)(Q₃₁), —S(═O)₂(Q₃₁), —P(Q₃₁)(Q₃₂), or —P(═O)(Q₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group; or
a C₃-C₆₀ carbocyclic group, a C₁-C₆₀ heterocyclic group, a C₇-C₆₀ aryl alkyl group, a C₂-C₆₀ heteroarylalkyl group, a C₆-C₆₀ aryloxy group, a C₆-C₆₀ arylthio group, a C₁-C₆₀ heteroaryloxy group, or a C₁-C₆₀ heteroarylthio group,
each unsubstituted or substituted with deuterium, —F, a cyano group, a C₁-C₆₀ alkyl group, a C₁-C₆₀ alkoxy group, a C₁-C₆₀ alkylthio group, a phenyl group, a biphenyl group, or a combination thereof, and
wherein Formula 1 includes an electron-withdrawing group (EWG).

In one or more embodiments, the EWG may include —F, —Cl, a cyano group, a nitro group, a carbonyl group, or a combination thereof. For example, Formula 1 may include a cyano group.

In one or more embodiments, the first electrode may be an anode, the second electrode may be a cathode, and the interlayer may further include a hole transport region arranged between the first electrode and the emission layer, wherein the hole transport region includes a hole injection layer, a hole transport layer, an electron-blocking layer, an emission auxiliary layer, or a combination thereof; and/or the interlayer may further include an electron transport region arranged between the second electrode and the emission layer, wherein the electron transport region includes a hole-blocking layer, an electron transport layer, an electron injection layer, or a combination thereof.

In one or more embodiments, the hole transport region may include the at least one compound represented by Formula 1.

In one or more embodiments, the hole injection layer may include the at least one compound represented by Formula 1. For example, the hole injection layer may include a p-dopant as the at least one compound represented by Formula 1.

In one or more embodiments, the emission layer may include a first host, a second host, and a dopant, and the dopant may include a compound including a ligand that includes a metal and an imidazole moiety.

In one or more embodiments, the first host may include a hole-transporting host.

In one or more embodiments, the second host may include an electron-transporting host.

The hole-transporting host may refer to a compound having strong hole transporting characteristics. The expression “a compound having strong hole transporting characteristics” refers to a compound that can readily accept holes, and such characteristics may be obtained by including a moiety that accepts holes well (i.e., a hole-transporting (HT) moiety).

The HT moiety may include, for example, a π electron-rich heteroaromatic compound (e.g., a carbazole derivative, an indole derivative, or the like), or an aromatic amine compound.

The electron-transporting host may refer to a compound having strong electron transporting characteristics. The expression “a compound having strong electron transporting characteristics” refers to a compound that can readily accept electrons, and such characteristics may be obtained by including a moiety that accepts electrons well (i.e., an electron-transporting (ET) moiety).

The ET moiety may include, for example, a Tr electron-deficient heteroaromatic compound. For example, the ET moiety may include a nitrogen-containing heteroaromatic compound.

When a compound includes only a HT moiety or only an ET moiety, it is clear to determine whether the nature of the compound has HT characteristics or ET characteristics.

In one or more embodiments, a compound may include both a HT moiety and an ET moiety. In this embodiment, a simple comparison between the total number of HT moieties and the total number of ET moieties present in the compound may be regarded as a criterion for predicting whether the compound is a HT compound or an ET compound, but it cannot be used as an absolute criterion. One reason for this is that a single HT moiety and a single ET moiety do not necessarily have exactly the same ability to attract holes and electrons, respectively.

Therefore, a relatively reliable method of determining whether a compound with a certain structure is an HT compound or an ET compound is to implement the compound directly in a device.

In one or more embodiments, a weight ratio of the first host to the second host may be about 9:1 to about 1:9. For example, the weight ratio of the first host to the second host may be about 6:4 to about 4:6. When the weight ratio of the first host to the second host is within the ranges above, the balance of injected charges may be appropriate.

In one or more embodiments, the metal of the dopant may include a transition metal.

For example, the dopant may include a compound represented by Formula 401:

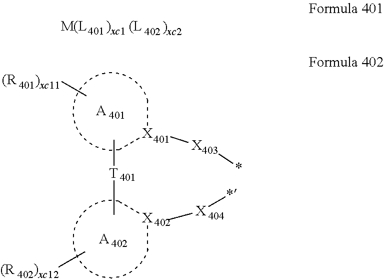

wherein, in Formulae 401 and 402,
M may be titanium (Ti), cobalt (Co), copper (Cu), zinc (Zn), zirconium (Zr), ruthenium (Ru), rhodium (Rh), palladium (Pd), platinum (Pt), gold (Au), osmium (Os), iridium (Ir), or rhenium (Re),
L₄₀₁ may be a ligand represented by Formula 402,
xc1 may be 1, 2, or 3, wherein, when xc1 is 2 or more, two or more of L₄₀₁ may be identical to or different from each other, two of ring A₄₀₁ may optionally be linked to each other via a linking group T₄₀₂, and two of ring A₄₀₂ may optionally be linked to each other via a linking group T₄₀₃,
L₄₀₂ may be an organic ligand,
xc2 may be 0, 1, 2, 3, or 4, wherein, when xc2 is 2 or more, two or more of L₄₀₂ may be identical to or different from each other,
X₄₀₁ and X₄₀₂ may each independently be nitrogen or carbon,
ring A₄₀₁ and ring A₄₀₂ may each independently a C₃-C₆₀ carbocyclic group or a C₁-C₆₀ heterocyclic group,
T₄₀₁ to T₄₀₃ may each independently be a single bond, —O—, —S—, —C(═O)—, —N(Q₄₁₁)-, —C(Q₄₁₁)(Q₄₁₂)-, —C(Q₄₁₁)═C(Q₄₁₂)-, —C(Q₄₁₁)═, or ═C═,
X₄₀₃ and X₄₀₄ may each independently be a chemical bond, —O—, —S—, —N(Q₄₁₃)-, —B(Q₄₁₃)-, —P(Q₄₁₃)-, —C(Q₄₁₃)(Q₄₁₄)-, or —Si(Q₄₁₃)(Q₄₁₄)-,
R₄₀₁ and R₄₀₂ may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group that is unsubstituted or substituted with at least one R₁₀ₐ, a C₁-C₂₀ alkoxy group that is unsubstituted or substituted with at least one R₁₀ₐ, a C₁-C₂₀ alkylthio group unsubstituted or substituted with at least one R₁₀ₐ, a C₃-C₁₀ carbocyclic group that is unsubstituted or substituted with at least one R₁₀ₐ, a C₁-C₆₀ heterocyclic group that is unsubstituted or substituted with at least one R₁₀ₐ, a C₇-C₆₀ alkyl aryl group unsubstituted or substituted with at least one R₁₀ₐ, a C₇-C₆₀ aryl alkyl group unsubstituted or substituted with at least one R₁₀ₐ, a C₂-C₆₀ alkyl heteroaryl group unsubstituted or substituted with at least one R₁₀ₐ, a C₂-C₆₀ heteroaryl alkyl group unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)(Q₄₀₁), —S(═O)₂(Q₄₀₁), —P(Q₄₀₁)(Q₄₀₂), or —P(═O)(Q₄₀₁)(Q₄₀₂),
optionally, R₄₀₁ and R₄₀₂ may be linked to each other to form a ring,
R₁₀ₐ may be:
deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, or a phosphoric acid group or a salt thereof;
a C₁-C₆₀ alkyl group, a C₂-C₆₀ alkenyl group, a 02-C₆₀ alkynyl group, a C₁-C₆₀ alkoxy group, or a C₁-C₆₀ alkylthio group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, a C₁-C₆₀ heteroaryloxy group, a C₁-C₆₀ heteroarylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)(Q₁₁), —S(═O)₂(Q₁₁), —P(Q₁₁)(Q₁₂), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, a C₁-C₆₀ heteroaryloxy group, a C₁-C₆₀ heteroarylthio group, a C₇-C₆₀ aryl alkyl group, or a C₂-C₆₀ heteroaryl alkyl group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a 02-C₆₀ heteroaryl alkyl group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O)(Q₂₁), —S(═O)(Q₂₁), —S(═O)₂(Q₂₁), —P(Q₂₁)(Q₂₂), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)(Q₃₁), —S(═O)₂(Q₃₁), —P(Q₃₁)(Q₃₂), or —P(═O)(Q₃₁)(Q₃₂), and
Q₁₁ to Q₁₃, Q₂₁ to Q₂₃, Q₃₁ to Q₃₃, Q₄₁₁ to Q₄₁₄, and Q₄₀₁ to Q₄₀₃ may each independently be: hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₇-C₆₀ aryl alkyl group, a C₂-C₆₀ heteroaryl alkyl group, a C₆-C₆₀ aryloxy group, a C₆-C₆₀ arylthio group, a C₁-C₆₀ heteroaryloxy group, or a C₁-C₆₀ heteroarylthio group, each unsubstituted or substituted with deuterium, —F, a cyano group, a C₁-C₆₀ alkyl group, a C₁-C₆₀ alkoxy group, a C₁-C₆₀ alkylthio group, a phenyl group, a biphenyl group, or a combination thereof;
xc11 and xc12 may each independently be an integer from 0 to 10,
* and *′ in Formula 402 each indicate a binding site to M in Formula 401, and
one of ring A₄₀₁ or ring A₄₀₂ may include an imidazole moiety.

For descriptions of the first host, the second host, and the dopant, reference may be made elsewhere herein.

In one or more embodiments, the compound of Formula 1 may be a symmetrical compound. For example, the compound of Formula 1 may be a line-symmetric compound or a point-symmetric compound.

In one or more embodiments, R₁ to R₄ in Formula 1 may each independently be a C₁-C₆₀ alkyl group unsubstituted or substituted with at least one R₁₀ₐ. For example, R₁ to R₄ in Formula 1 may each independently be an unsubstituted methyl group, an unsubstituted ethyl group, an unsubstituted n-propyl group, an unsubstituted iso-propyl group, an unsubstituted n-butyl group, an unsubstituted sec-butyl group, or an unsubstituted tert-butyl group.

In one or more embodiments, R₅ to R₈ in Formula 1 may each independently be a C₆-C₆₀ aryl group unsubstituted or substituted with at least one R₁₀ₐ. For example, R₅ to R₈ in Formula 1 may each independently be a phenyl group unsubstituted or substituted with at least one R₁₀ₐ, a naphthyl group unsubstituted or substituted with at least one R₁₀ₐ, a phenanthrenyl group unsubstituted or substituted with at least one R₁₀ₐ, an anthracenyl group unsubstituted or substituted with at least one R₁₀ₐ, or a pyrenyl group unsubstituted or substituted with at least one R₁₀ₐ.

In one or more embodiments, R₁ to R₄ in Formula 1 may each include an EWG. For example, R₁ to R₄ may each independently include —F, —Cl, a cyano group, a nitro group, or a carbonyl group. For example, R₁ to R₄ may each independently be —F, —Cl, a cyano group, or a nitro group.

In one or more embodiments, the compound represented by Formula 1 may include any one of the following compounds:

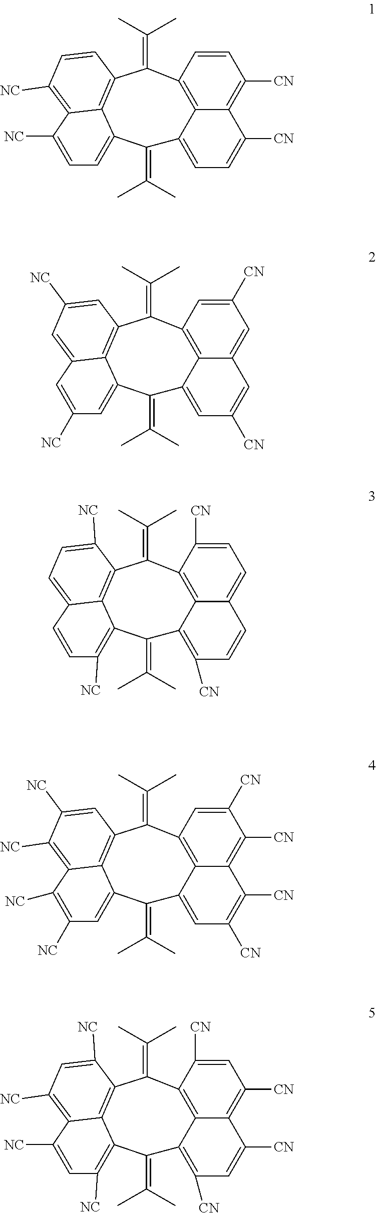 [SMILES: CC(C)=c1c2ccc(C#N)c3c(C#N)ccc(c(=C(C)C)c4ccc(C#N)c5c(C#N)ccc1c54)c32 ; CC(C)=c1c2cc(C#N)cc3cc(C#N)cc(c(=C(C)C)c4cc(C#N)cc5cc(C#N)cc1c54)c32 ; CC(C)=c1c2c(C#N)ccc3ccc(C#N)c(c(=C(C)C)c4c(C#N)ccc5ccc(C#N)c1c54)c32 ; CC(C)=c1c2cc(C#N)c(C#N)c3c(C#N)c(C#N)cc(c(=C(C)C)c4cc(C#N)c(C#N)c5c(C#N)c(C#N)cc1c54)c32 ; CC(C)=c1c2c(C#N)cc(C#N)c3c(C#N)cc(C#N)c(c(=C(C)C)c4c(C#N)cc(C#N)c5c(C#N)cc(C#N)c1c54)c32]

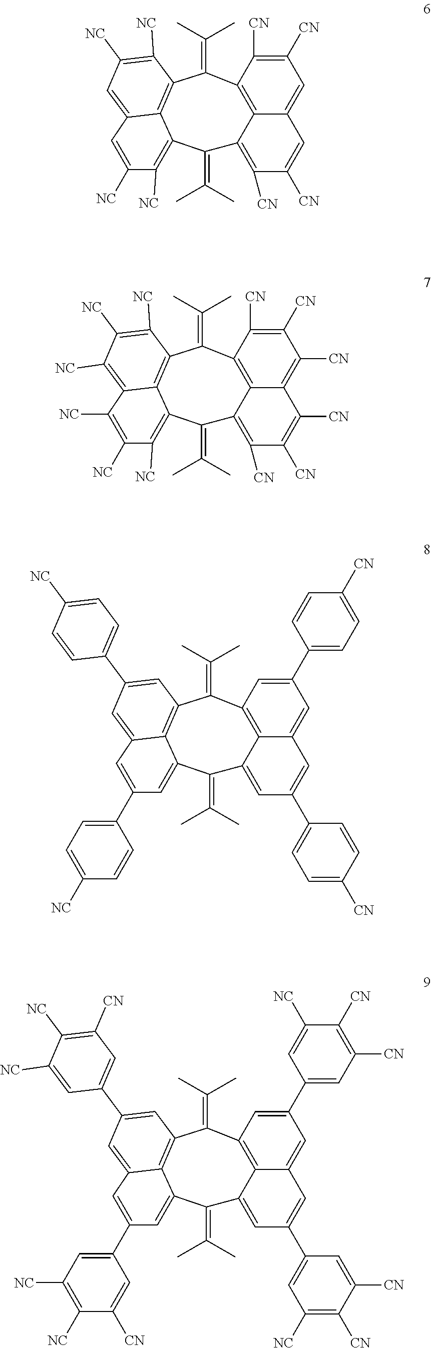 [SMILES: CC(C)=c1c2c(C#N)c(C#N)cc3cc(C#N)c(C#N)c(c(=C(C)C)c4c(C#N)c(C#N)cc5cc(C#N)c(C#N)c1c54)c32 ; CC(C)=c1c2c(C#N)c(C#N)c(C#N)c3c(C#N)c(C#N)c(C#N)c(c(=C(C)C)c4c(C#N)c(C#N)c(C#N)c5c(C#N)c(C#N)c(C#N)c1c54)c32 ; CC(C)=c1c2cc(-c3ccc(C#N)cc3)cc3cc(-c4ccc(C#N)cc4)cc(c(=C(C)C)c4cc(-c5ccc(C#N)cc5)cc5cc(-c6ccc(C#N)cc6)cc1c54)c32 ; CC(C)=c1c2cc(-c3cc(C#N)c(C#N)c(C#N)c3)cc3cc(-c4cc(C#N)c(C#N)c(C#N)c4)cc(c(=C(C)C)c4cc(-c5cc(C#N)c(C#N)c(C#N)c5)cc5cc(-c6cc(C#N)c(C#N)c(C#N)c6)cc1c54)c32]

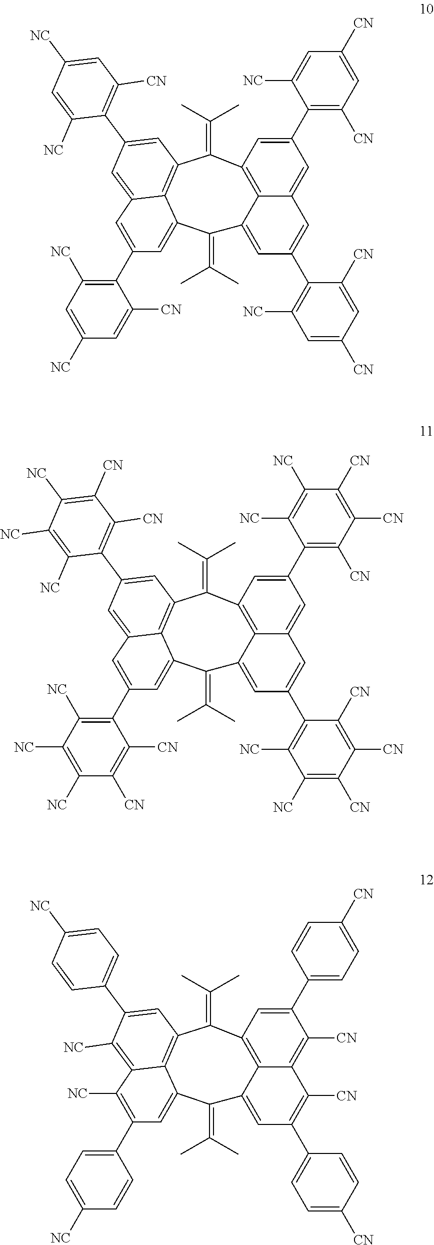 [SMILES: CC(C)=c1c2cc(-c3c(C#N)cc(C#N)cc3C#N)cc3cc(-c4c(C#N)cc(C#N)cc4C#N)cc(c(=C(C)C)c4cc(-c5c(C#N)cc(C#N)cc5C#N)cc5cc(-c6c(C#N)cc(C#N)cc6C#N)cc1c54)c32 ; CC(C)=c1c2cc(-c3c(C#N)c(C#N)c(C#N)c(C#N)c3C#N)cc3cc(-c4c(C#N)c(C#N)c(C#N)c(C#N)c4C#N)cc(c(=C(C)C)c4cc(-c5c(C#N)c(C#N)c(C#N)c(C#N)c5C#N)cc5cc(-c6c(C#N)c(C#N)c(C#N)c(C#N)c6C#N)cc1c54)c32 ; CC(C)=c1c2cc(-c3ccc(C#N)cc3)c(C#N)c3c(C#N)c(-c4ccc(C#N)cc4)cc(c(=C(C)C)c4cc(-c5ccc(C#N)cc5)c(C#N)c5c(C#N)c(-c6ccc(C#N)cc6)cc1c54)c32]

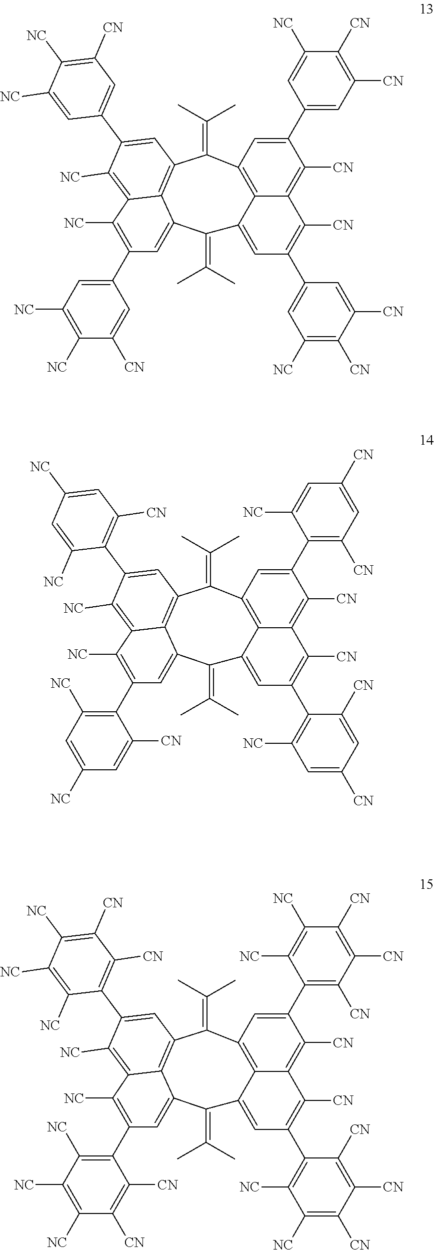

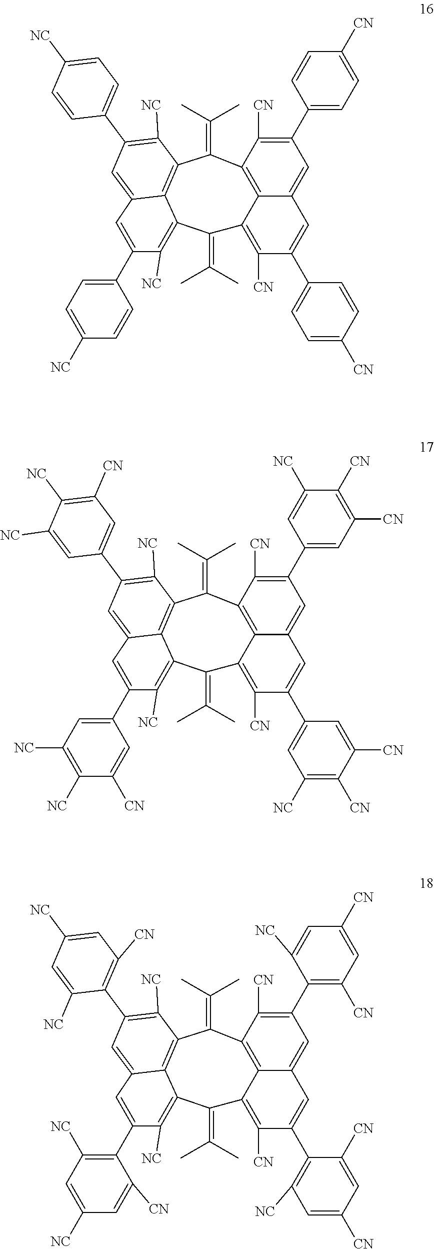 [SMILES: CC(C)=c1c2c(C#N)c(-c3ccc(C#N)cc3)cc3cc(-c4ccc(C#N)cc4)c(C#N)c(c(=C(C)C)c4c(C#N)c(-c5ccc(C#N)cc5)cc5cc(-c6ccc(C#N)cc6)c(C#N)c1c54)c32 ; CC(C)=c1c2c(C#N)c(-c3cc(C#N)c(C#N)c(C#N)c3)cc3cc(-c4cc(C#N)c(C#N)c(C#N)c4)c(C#N)c(c(=C(C)C)c4c(C#N)c(-c5cc(C#N)c(C#N)c(C#N)c5)cc5cc(-c6cc(C#N)c(C#N)c(C#N)c6)c(C#N)c1c54)c32 ; CC(C)=c1c2c(C#N)c(-c3c(C#N)cc(C#N)cc3C#N)cc3cc(-c4c(C#N)cc(C#N)cc4C#N)c(C#N)c(c(=C(C)C)c4c(C#N)c(-c5c(C#N)cc(C#N)cc5C#N)cc5cc(-c6c(C#N)cc(C#N)cc6C#N)c(C#N)c1c54)c32]

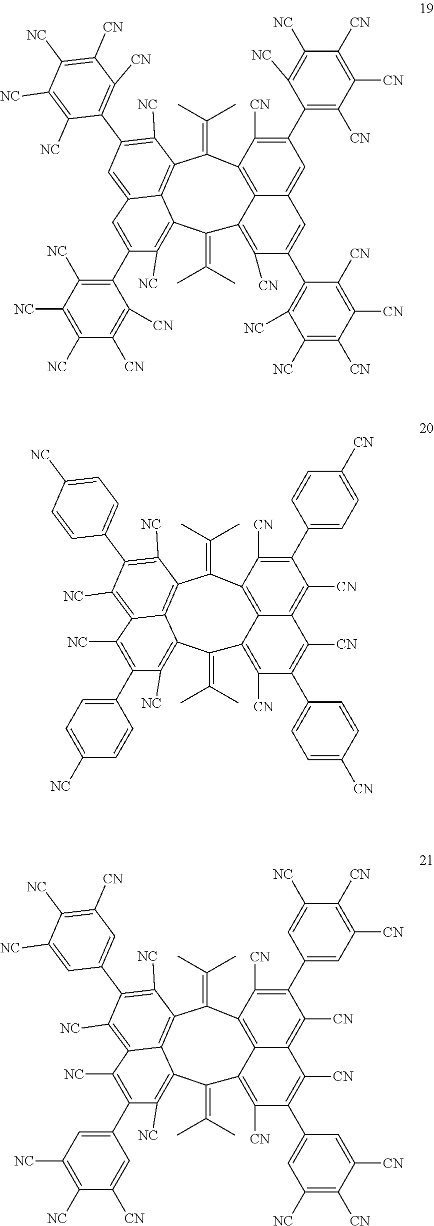

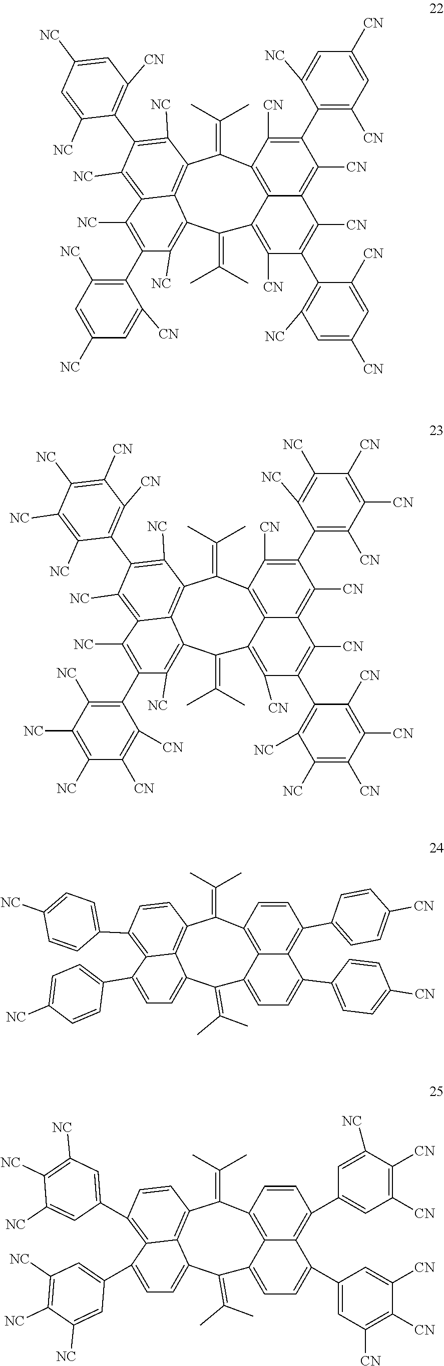

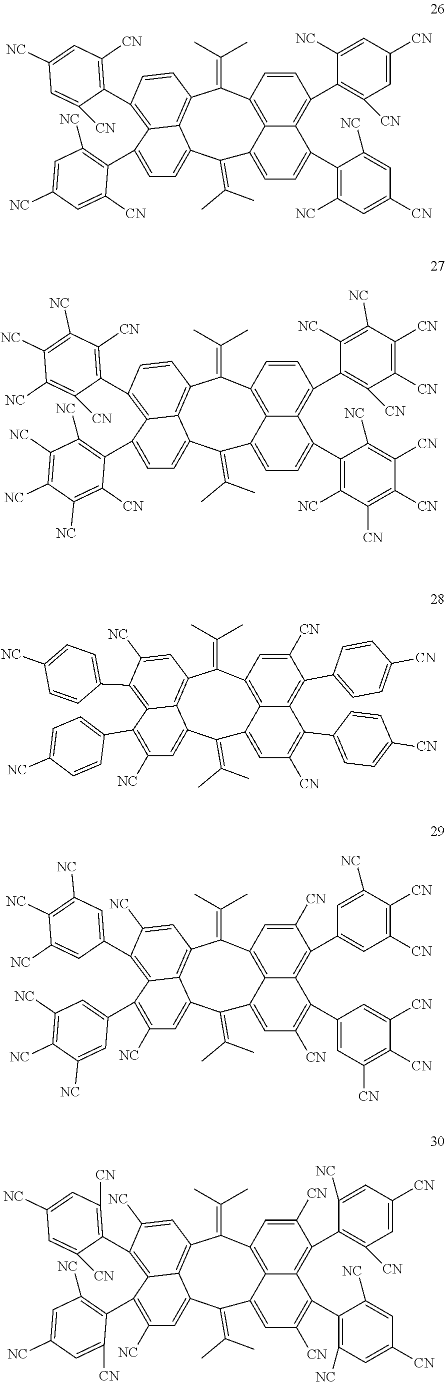

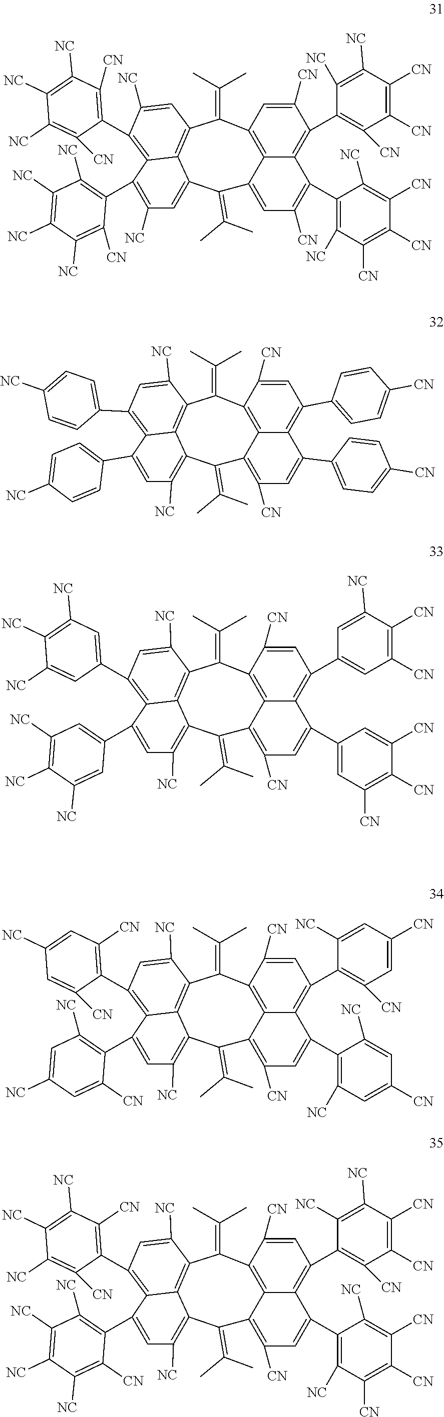

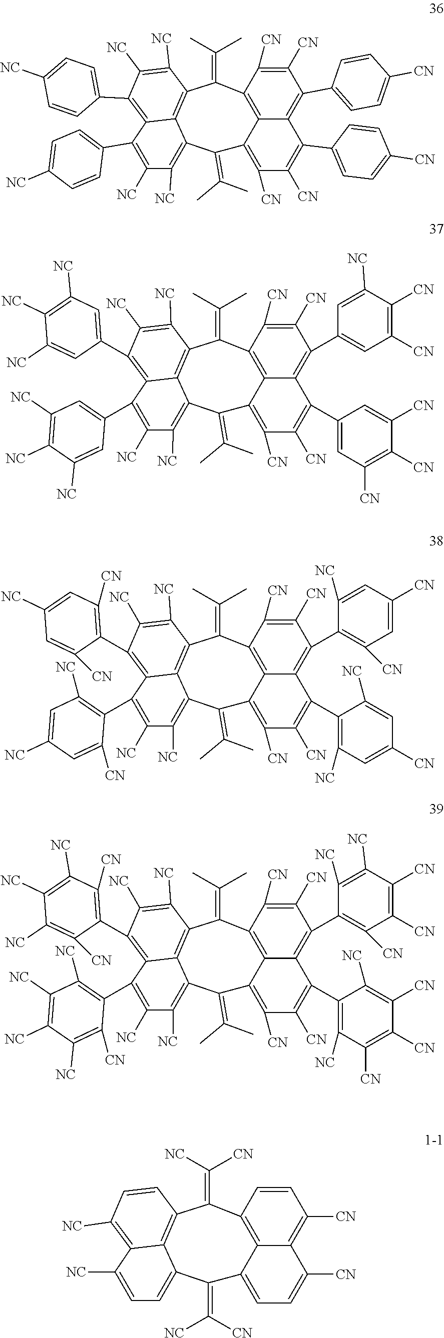

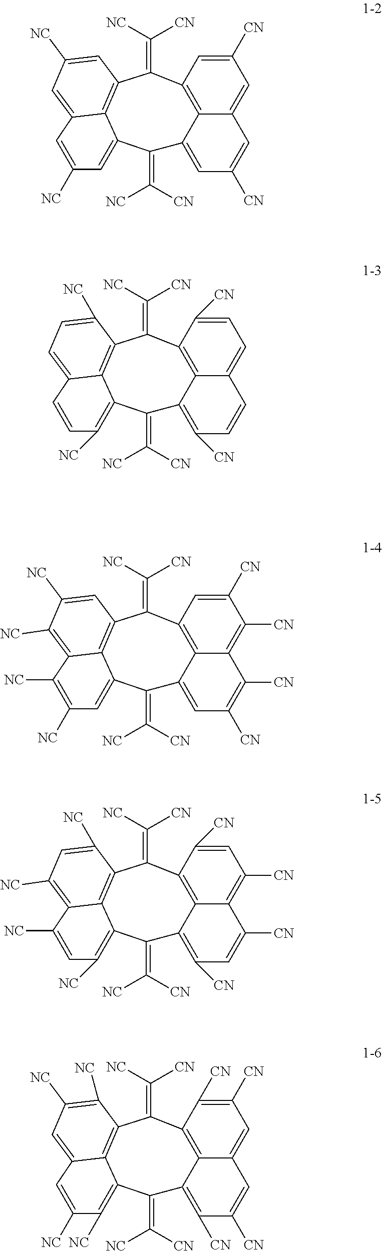 [SMILES: N#CC(C#N)=c1c2cc(C#N)cc3cc(C#N)cc(c(=C(C#N)C#N)c4cc(C#N)cc5cc(C#N)cc1c54)c32 ; N#CC(C#N)=c1c2c(C#N)ccc3ccc(C#N)c(c(=C(C#N)C#N)c4c(C#N)ccc5ccc(C#N)c1c54)c32 ; N#CC(C#N)=c1c2cc(C#N)c(C#N)c3c(C#N)c(C#N)cc(c(=C(C#N)C#N)c4cc(C#N)c(C#N)c5c(C#N)c(C#N)cc1c54)c32 ; N#CC(C#N)=c1c2c(C#N)cc(C#N)c3c(C#N)cc(C#N)c(c(=C(C#N)C#N)c4c(C#N)cc(C#N)c5c(C#N)cc(C#N)c1c54)c32 ; N#CC(C#N)=c1c2c(C#N)c(C#N)cc3cc(C#N)c(C#N)c(c(=C(C#N)C#N)c4c(C#N)c(C#N)cc5cc(C#N)c(C#N)c1c54)c32]

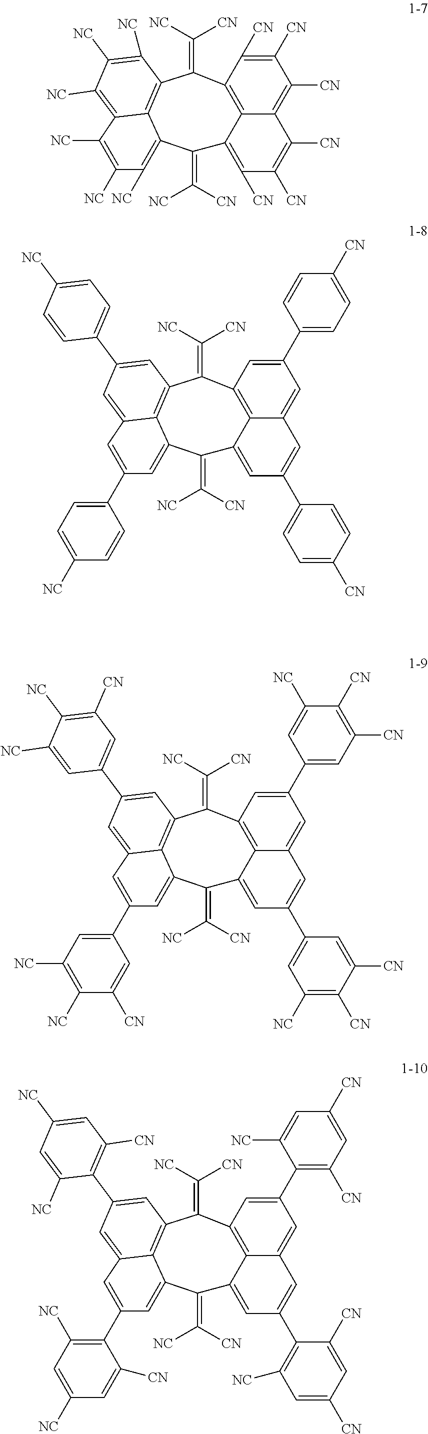

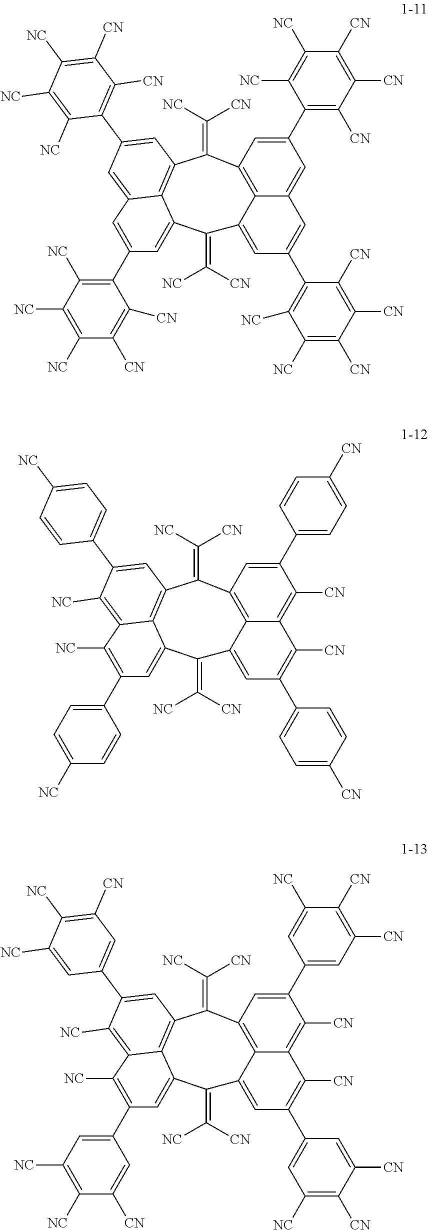

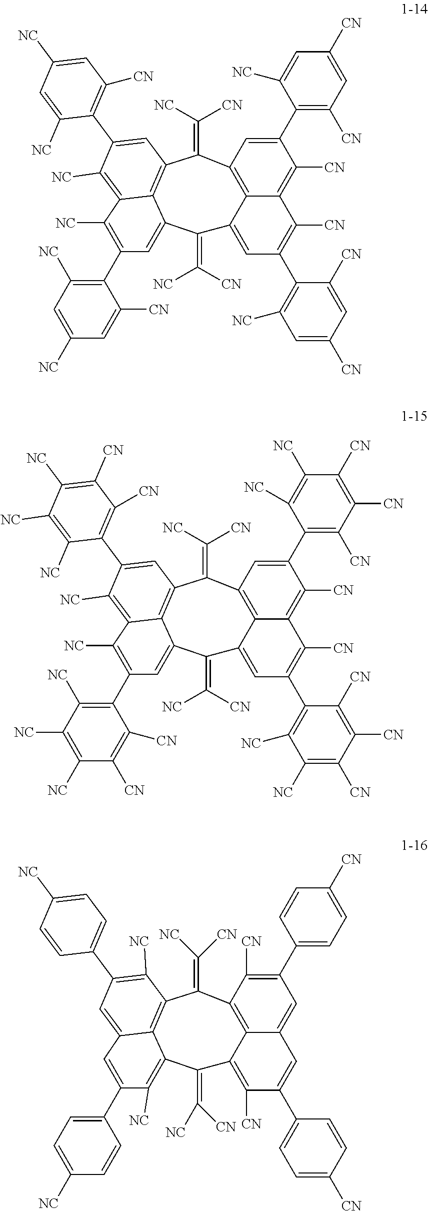

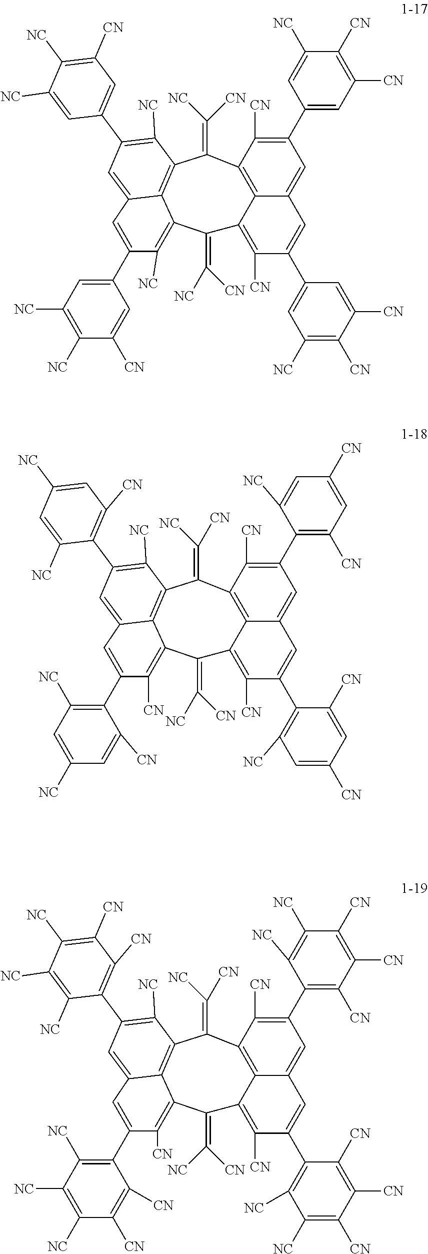

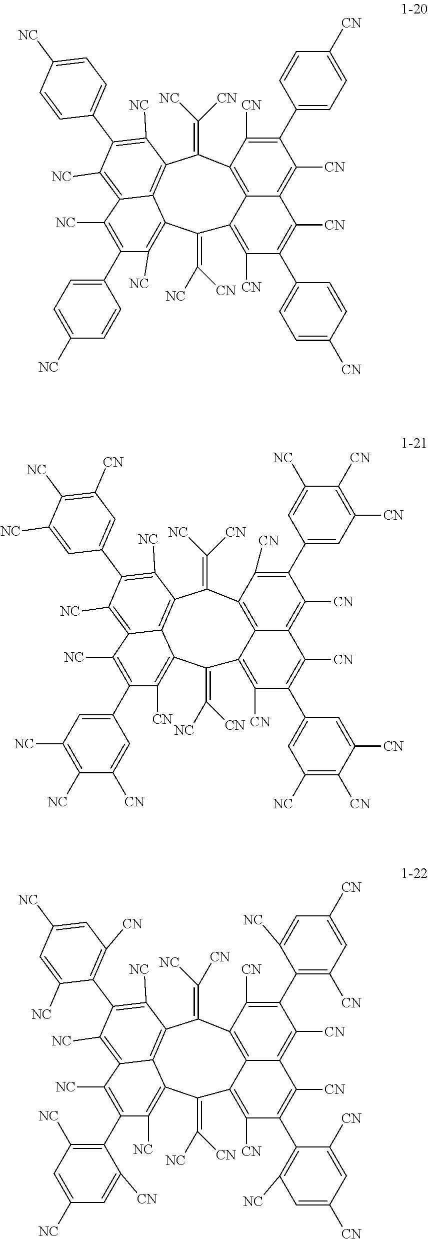

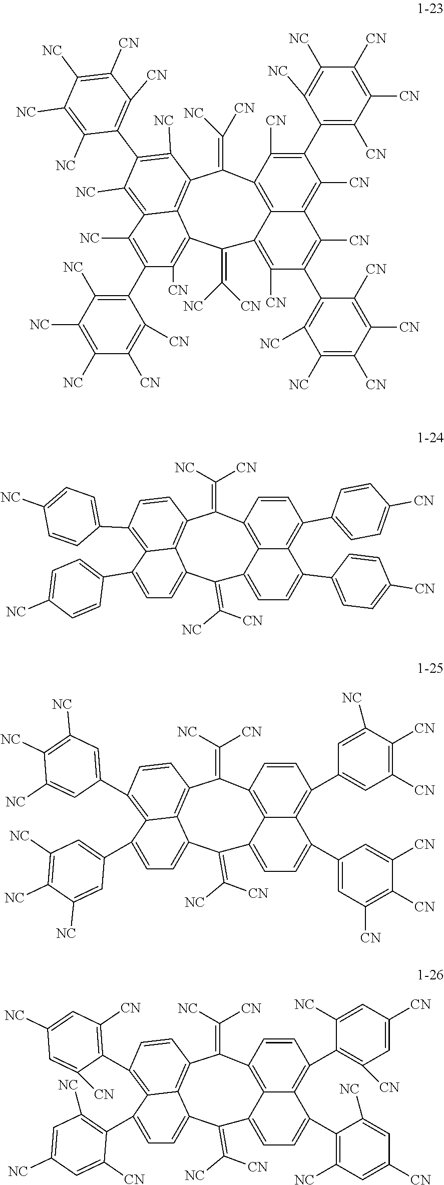

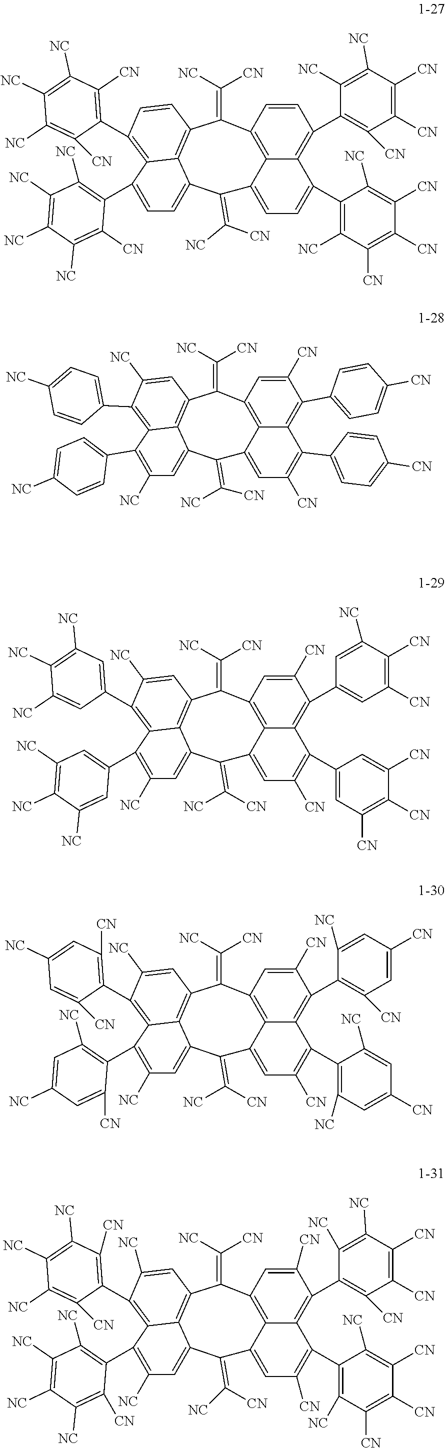

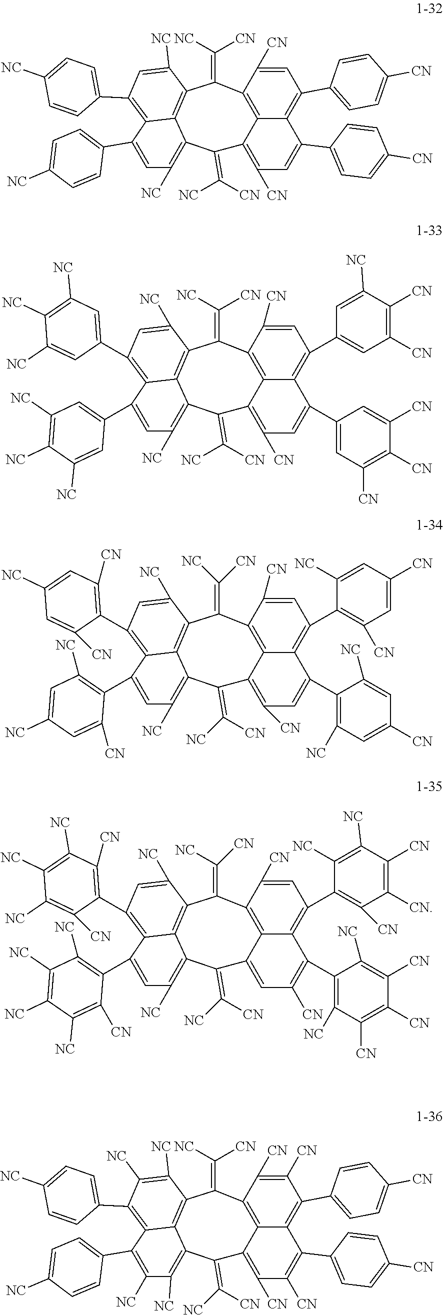

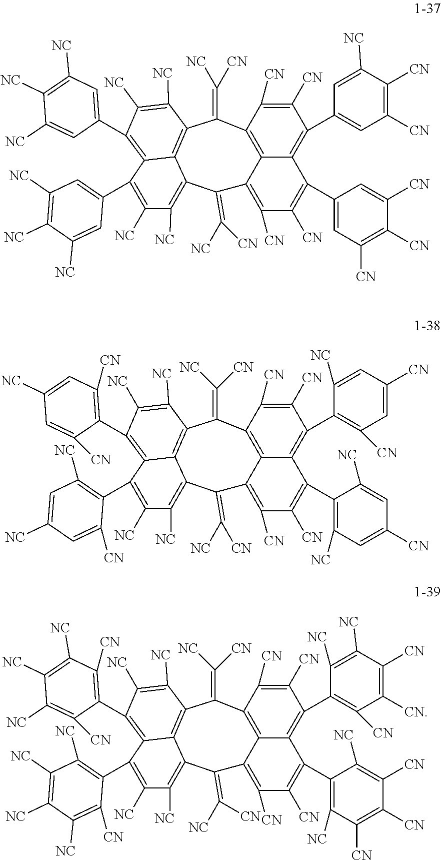

In one or more embodiments, the emission layer may be a phosphorescent emission layer.

In one or more embodiments, the emission layer may be a blue emission layer.

In one or more embodiments, the interlayer may include a red mission layer, a blue emission layer, and/or a green emission layer. For example, the interlayer may include the red emission layer, the blue emission layer, and/or the green emission layer in series or in parallel on the same plane. For example, the interlayer may include the red emission layer, the blue emission layer, the green emission layer, and the hole injection layer, wherein the hole injection layer includes at least one of the compounds represented by Formula 1.

In one or more embodiments, the emission layer may include an emission layer in the number of m.

The interlayer may further include m-1 charge generation layer(s) each arranged between two neighboring emission layers among the emission layers in the number of m,
wherein m may be an integer of 2 or more, and
the charge generation layer may include the at least one compound represented by Formula 1.

For example, the charge generation layer may include an n-charge generation layer and a p-charge generation layer. For example, the p-charge generation layer may include the at least one compound represented by Formula 1.

Referring to FIG. 2, an interlayer 130 may include emission layers in the number of m 145(1), 145(m-1)(not shown), and 145(m), and m-1 charge generation layer(s) 144(m-1) each arranged between neighboring emission layers.

In one or more embodiments, when m is 2, a first electrode, a first emission layer, a first charge generation layer, and a second emission layer may be sequentially arranged. In this embodiment, the first emission layer may emit a first-color light, and the second emission layer may emit a second-color light, wherein a maximum emission wavelength of the first-color light and a maximum emission wavelength of the second-color light may be identical to or different from each other.

In another embodiment, when m is 3, a first electrode, a first emission layer, a first charge generation layer, a second emission layer, a second charge generation layer, and a third emission layer may be sequentially arranged. In this embodiment, the first emission layer may emit a first-color light, the second emission may emit a second-color light, and the third emission layer may emit a third-color light, wherein a maximum emission wavelength of the first-color light, a maximum emission wavelength of the second-color light, and a maximum emission wavelength of the third-color light may be identical to or different from one another.

In another embodiment, when m is 4, a first electrode, a first emission layer, a first charge generation layer, a second emission layer, a second charge generation layer, a third emission layer, a third charge generation layer, and a fourth emission layer may be sequentially arranged. In this embodiment, the first emission layer may emit a first-color light, the second emission layer may emit a second-color light, the third emission layer may emit a third-color light, and the fourth emission layer may emit a fourth-color light, wherein a maximum emission wavelength of the first-color light, a maximum emission wavelength of the second-color light, a maximum emission wavelength of the third-color light, and a maximum emission wavelength of the fourth-color light may be identical to or different from one another.

The same applies for m being an integer from 5 to 7.

Another aspect provides an electronic apparatus including the light-emitting device. The electronic apparatus may further include a thin-film transistor. In one or more embodiments, the electronic apparatus may further include a thin-film transistor including a source electrode and a drain electrode, wherein the first electrode of the light-emitting device may be electrically connected to at least one of the source electrode or the drain electrode.

In another embodiment, the electronic apparatus may further include a color filter, a color conversion layer, a touch screen layer, a polarizing layer, or a combination thereof. More details on the electronic apparatus may be referred to the descriptions provided herein.

The term “interlayer” as used herein refers to a single layer and/or multiple layers arranged between the first electrode and the second electrode of the light-emitting device.

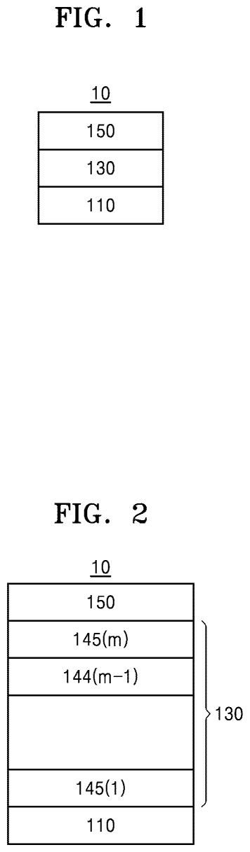

### Description of FIG. 1

FIG. 1 is a schematic cross-sectional view of a light-emitting device 10 according to one or more embodiments. The light-emitting device 10 includes a first electrode 110, an interlayer 130, and a second electrode 150.

Hereinafter, a structure of the light-emitting device 10 according to one or more embodiments and a method of manufacturing the light-emitting device 10 are described with reference to FIG. 1.

### First Electrode 110

In FIG. 1, a substrate may be additionally arranged under the first electrode 110 or on the second electrode 150. As the substrate, a glass substrate or a plastic substrate may be used. In one or more embodiments, the substrate may be a flexible substrate and may include a plastic with excellent heat resistance and durability, such as polyimide, polyethylene terephthalate (PET), polycarbonate, polyethylene naphthalate, polyarylate (PAR), polyetherimide, or a combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a transflective electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or a combination thereof. In one or more embodiments, when the first electrode 110 is a transflective electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or a combination thereof.

The first electrode 110 may have a single-layer structure consisting of a single layer or a multilayer structure including a plurality of layers. In one or more embodiments, the first electrode 110 may have a three-layer structure of ITO/Ag/ITO.

### Interlayer 130

The interlayer 130 is arranged on the first electrode 110. The interlayer 130 includes an emission layer.

The interlayer 130 may further include a hole transport region arranged between the first electrode 110 and the emission layer, and an electron transport region arranged between the emission layer and the second electrode 150.

The interlayer 130 may further include, in addition to various organic materials, a metal-containing compound such as an organometallic compound, an inorganic material such as quantum dots, or the like.

In one or more embodiments, the interlayer 130 may include i) two or more emitting units sequentially stacked between the first electrode 110 and the second electrode 150, and ii) a charge generation layer arranged between neighboring two emitting units. When the interlayer 130 includes the emission layers and the charge generation layer as described above, the light-emitting device 10 may be a tandem light-emitting device.

### Hole Transport Region in Interlayer 130

The hole transport region may have: i) a single-layer structure consisting of a single layer consisting of a single material, ii) a single-layer structure consisting of a single layer consisting of a plurality of materials that are different from each other, or iii) a multilayer structure including a plurality of layers including a plurality of materials that are different from each other.

The hole transport region may include a hole injection layer, a hole transport layer, an emission auxiliary layer, an electron-blocking layer, or a combination thereof.

In one or more embodiments, the hole transport region may have a multilayer structure including a hole injection layer/hole transport layer structure, a hole injection layer/hole transport layer/emission auxiliary layer structure, a hole injection layer/emission auxiliary layer structure, or a hole injection layer/hole transport layer/electron-blocking layer structure, wherein the layers of each structure are sequentially stacked from the first electrode 110 in the stated order.

For example, the hole transport region may have a multilayer structure of hole injection layer/hole transport layer/emission auxiliary layer, or hole injection layer/hole transport layer/electron-blocking layer, wherein the layers of each structure are sequentially stacked from the first electrode 110 in the stated order.

The hole transport region may include a compound represented by Formula 201, a compound represented by Formula 202, or a combination thereof, but embodiments are not limited thereto:

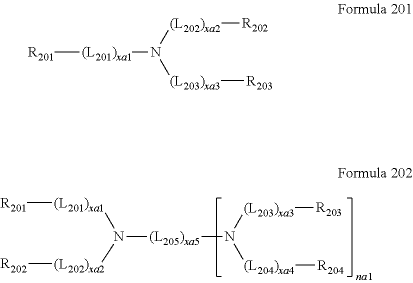

wherein, in Formulae 201 and 202,
L₂₀₁ to L₂₀₄ may each independently be a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
L₂₀₅ may be *—O—*′, *—S—*, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
R₂₀₁ and R₂₀₂ may optionally be linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group (for example, a carbazole group or the like) that is unsubstituted or substituted with at least one R₁₀ₐ (for example, Compound HT16 or the like),
R₂₀₃ and R₂₄ may optionally be linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group that is unsubstituted or substituted with at least one R₁₀ₐ, and
na1 may be an integer from 1 to 4.

In one or more embodiments, each of Formulae 201 and 202 may include at least one of groups represented by Formulae CY201 to CY217:

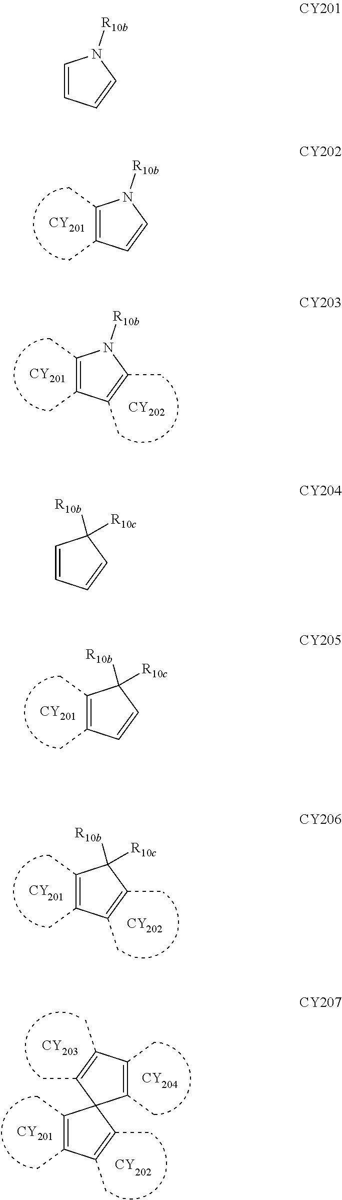

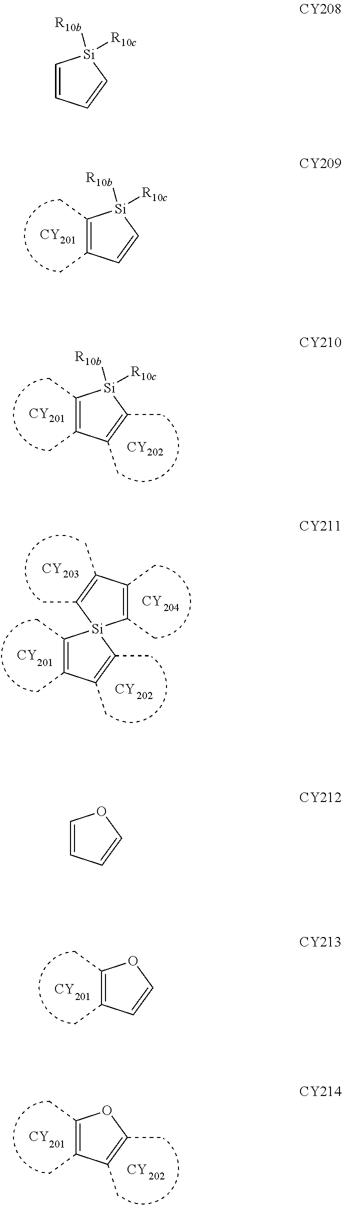

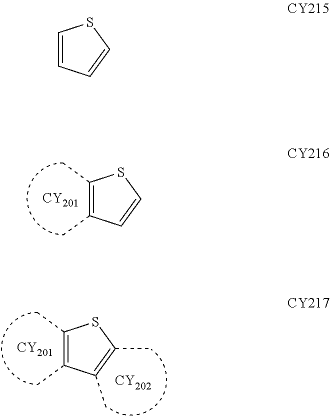

wherein, in Formulae CY201 to CY217, R_{10b} and R_{10c} may each be as described herein in connection with R₁₀ₐ, ring CY21 to ring CY204 may each independently be a C₃-C₂₀ carbocyclic group or a C₁-C₂₀ heterocyclic group, and at least one hydrogen in Formulae CY201 to CY217 may be substituted with R₁₀ₐ.

In one or more embodiments, ring CY201 to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one of the groups represented by Formulae CY201 to CY203.

In one or more embodiments, Formula 201 may include at least one of the groups represented by Formulae CY201 to CY203 and at least one of the groups represented by Formulae CY204 to CY217.

In one or more embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more embodiments, each of Formulae 201 and 202 may not include the groups represented by Formulae CY201 to CY203.

In one or more embodiments, each of Formulae 201 and 202 may not include the groups represented by Formulae CY201 to CY203 and may include at least one of the groups represented by Formulae CY204 to CY217.

In one or more embodiments, each of Formulae 201 and 202 may not include the groups represented by Formulae CY201 to CY217.

In one or more embodiments, the hole transport region may include one of Compounds HT1 to HT46, 4,4′,4″-tris(3-methylphenylphenylamino)triphenylamine (m-MTDATA), 4,4′,4″-tris(N,N-diphenylamino)triphenylamine (TDATA), 4,4′,4″-tris{N-(2-naphthyl)-N-phenylamino}-triphenylamine (2-TNATA), N,N′-di(1-naphthyl)-N,N′-diphenylbenzidine (NPB), β-NPB, N,N′-bis(3-methylphenyl)-N,N′-diphenyl-[1, 1-biphenyl]-4,4′-diamine (TPD), Spiro-TPD, Spiro-NPB, methylated NPB, 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or a combination thereof, but embodiments are not limited thereto:

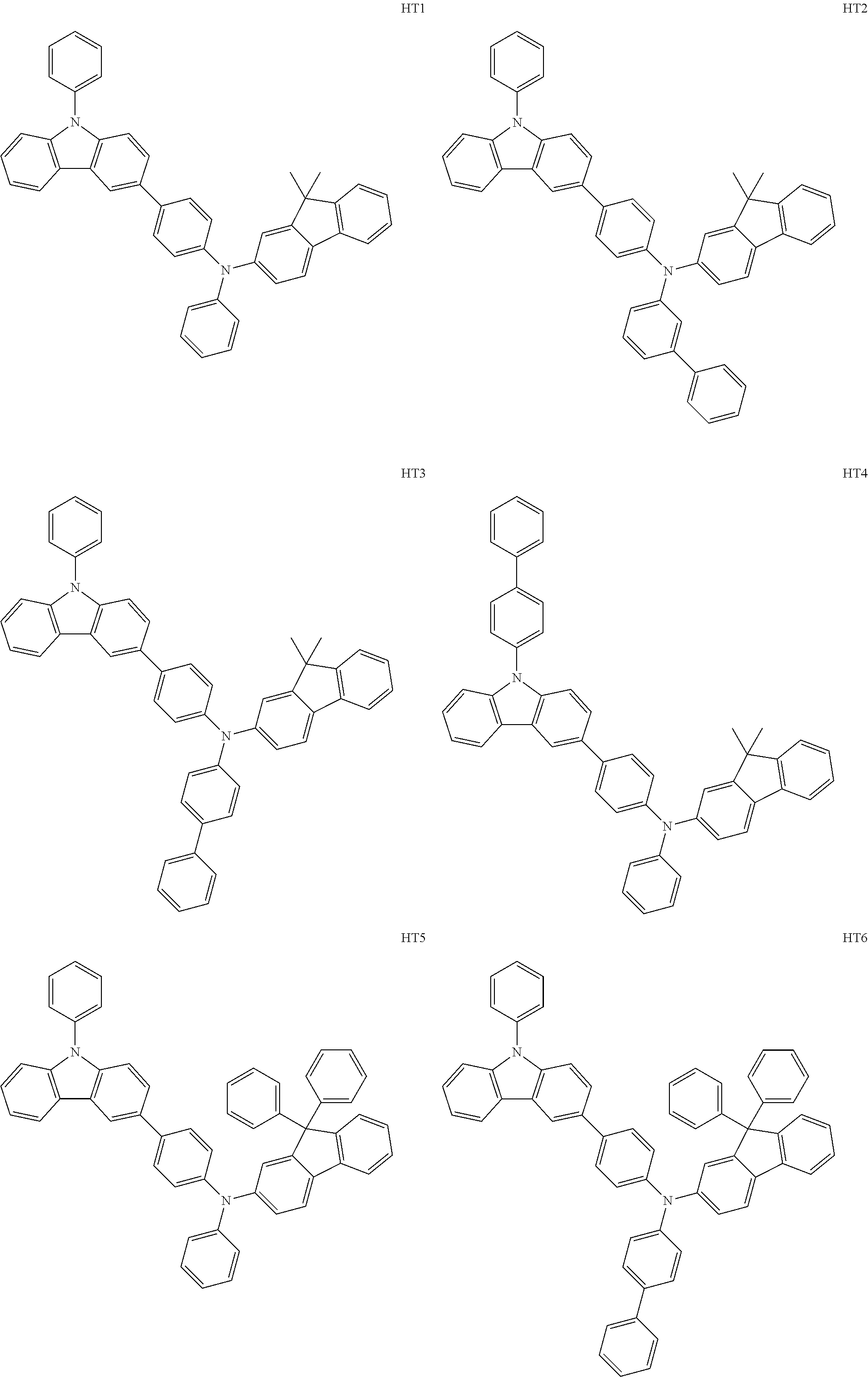

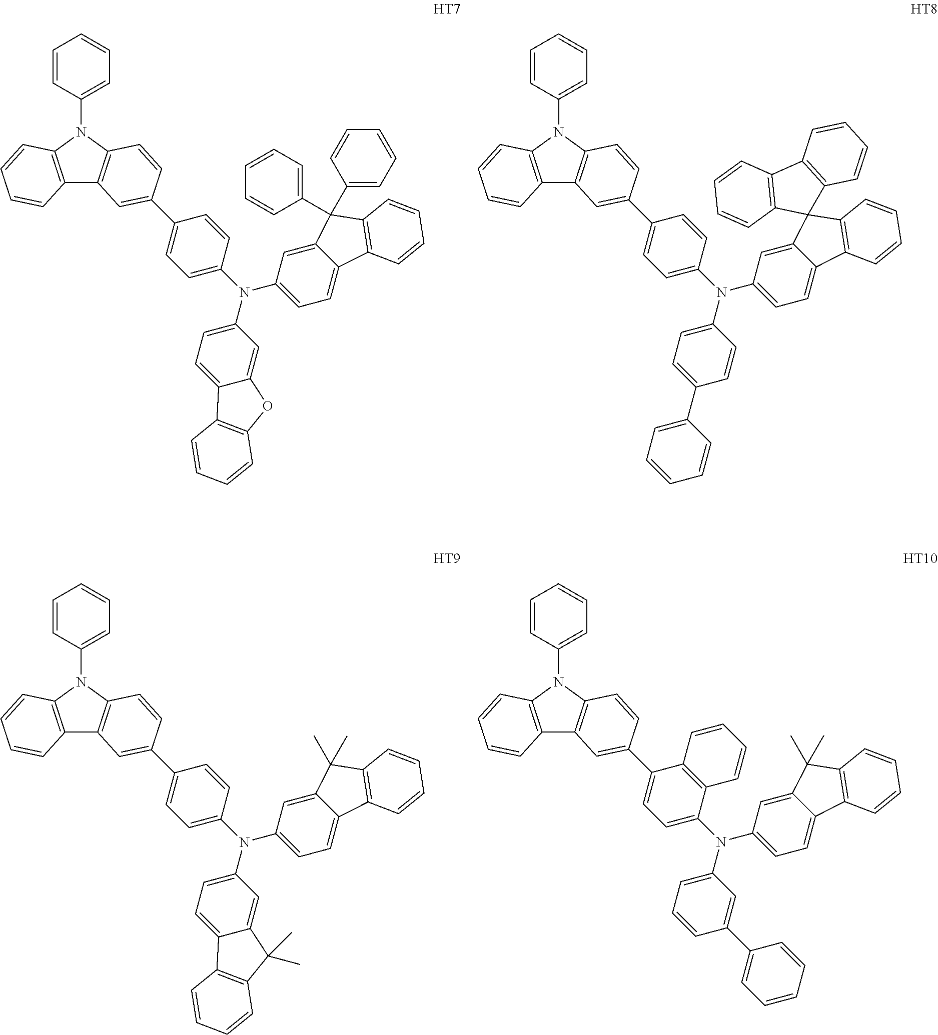 [SMILES: c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21]

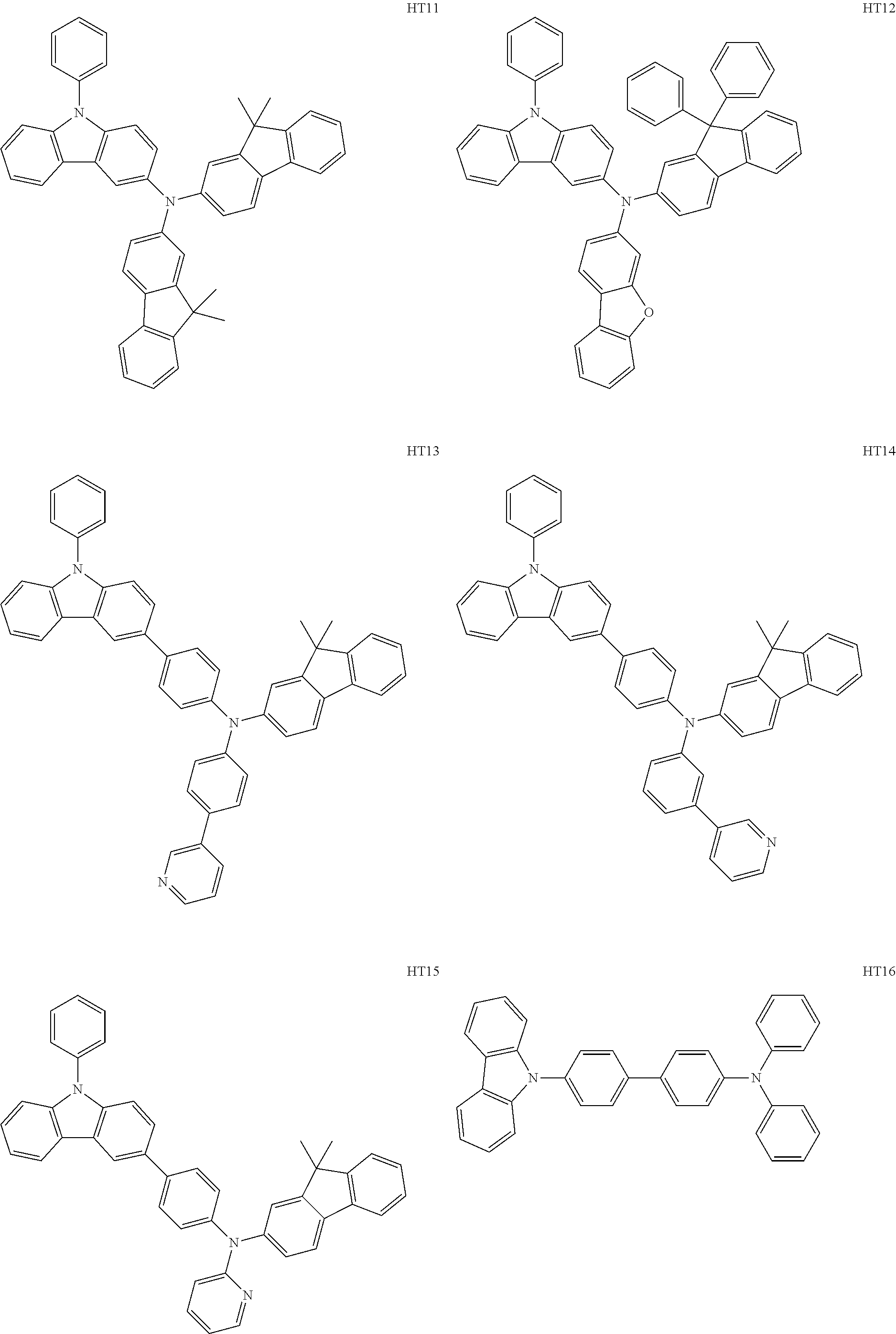

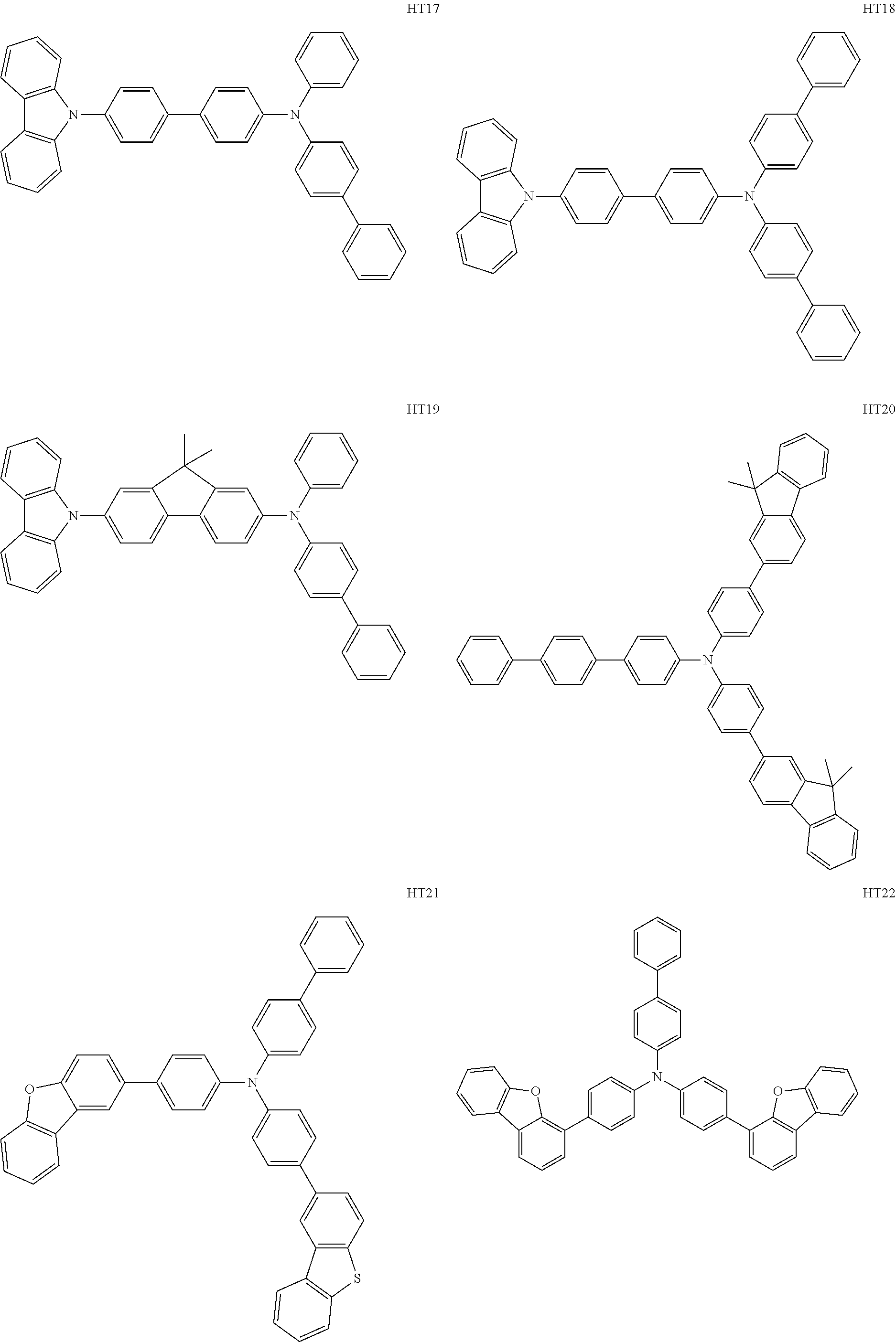

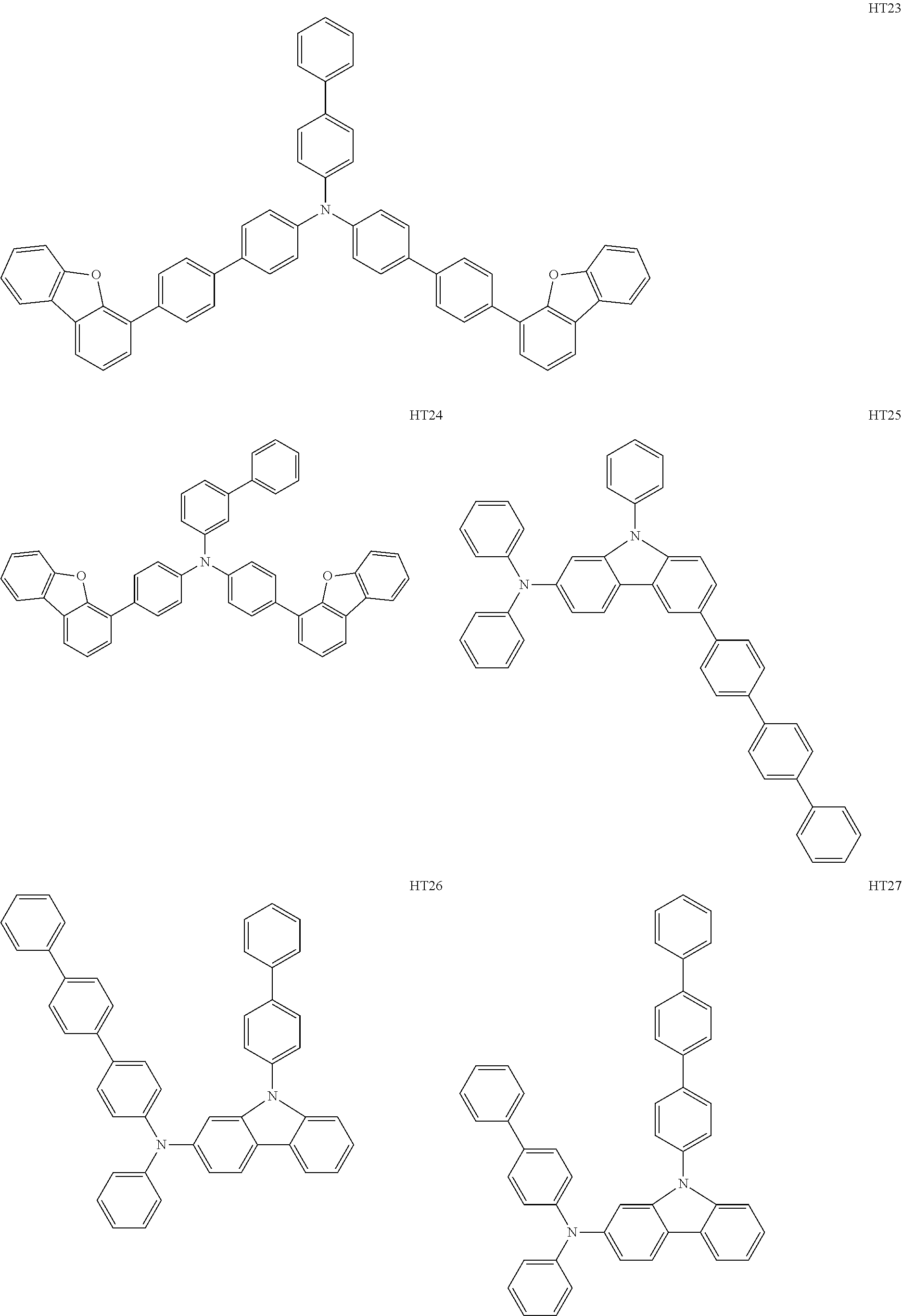 [SMILES: c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

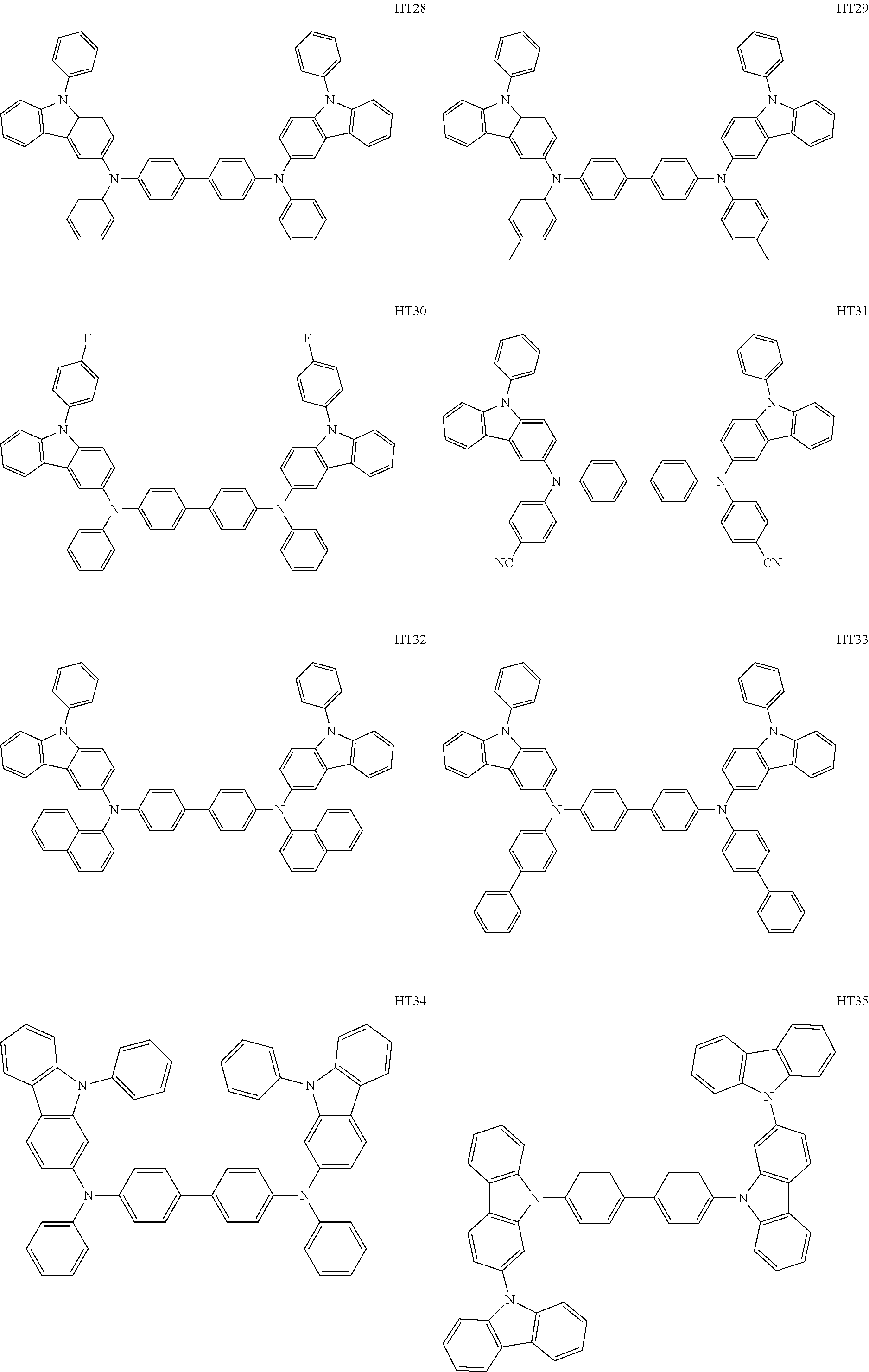

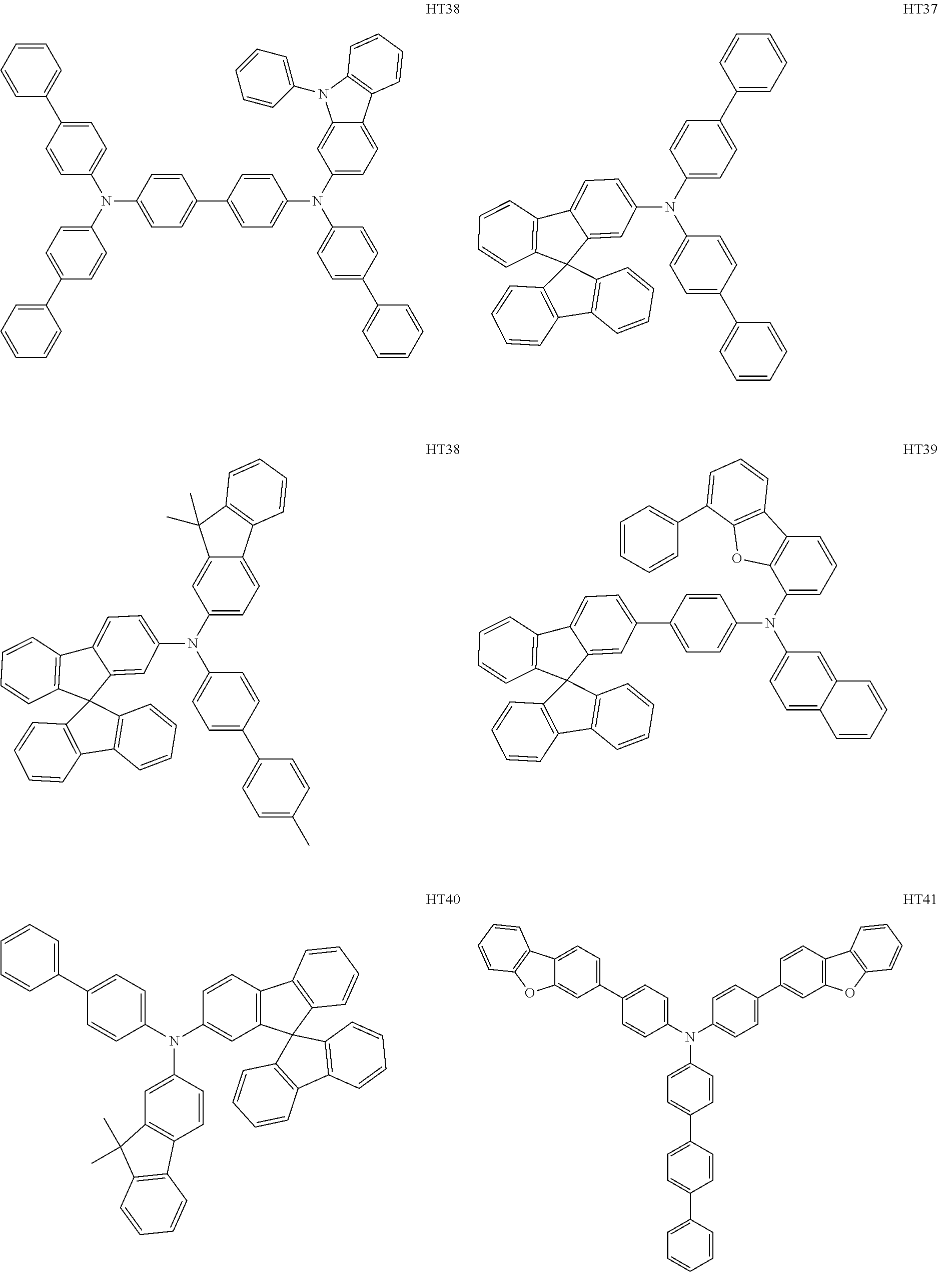

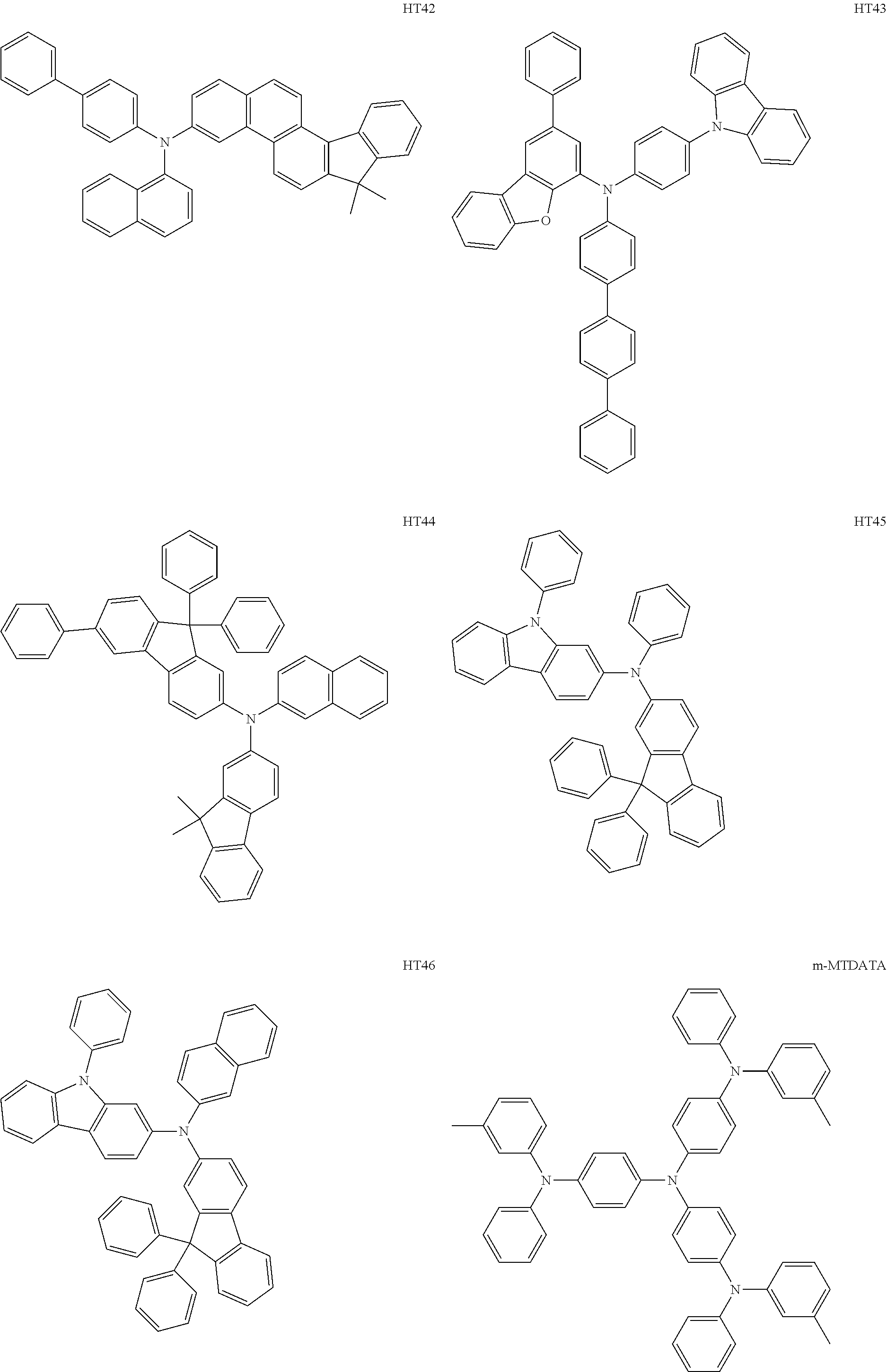

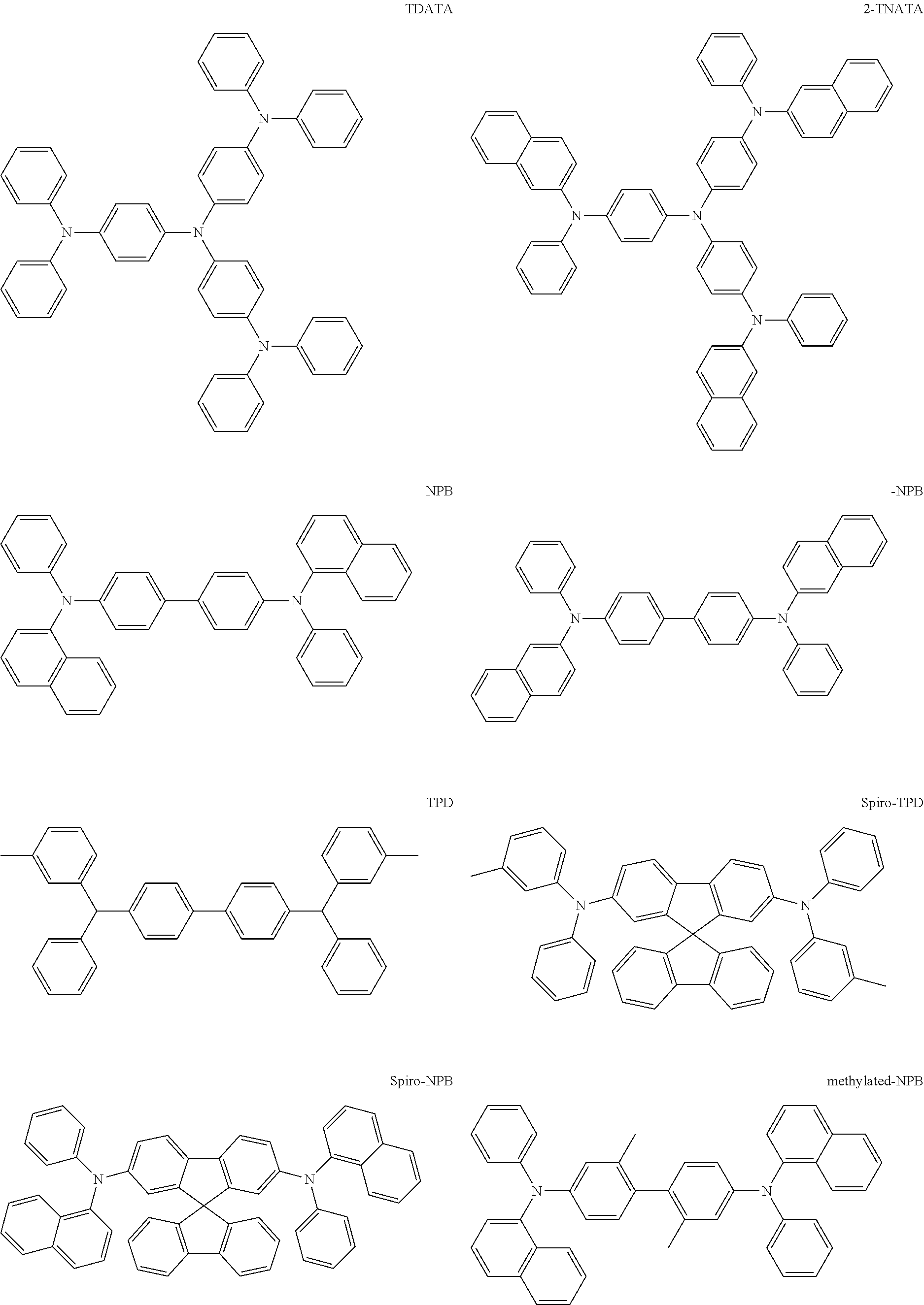

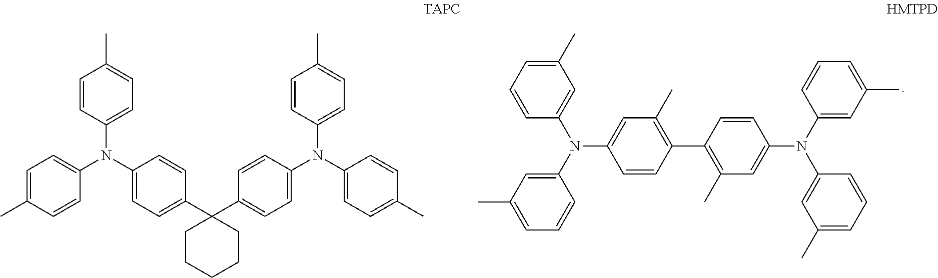

A thickness of the hole transport region may be about 50 Å to about 10,000 Å, for example, about 100 Å to about 4,000 Å. A thickness of the hole injection layer may be about 10 Å to about 1,000 Å, for example, about 20 Å to about 500 Å. When the thickness of the hole injection layer is within the ranges above, satisfactory hole injection characteristics may be obtained without a substantial increase in driving voltage.

In one or more embodiments, the hole injection layer may include at least one of the compounds represented by Formula 1 as a p-dopant.

A doping ratio of the p-dopant may be greater than 0% and less than 50% (weight percent, wt %). For example, the doping ratio may be greater than 0% and up to 30% (wt %). For example, the doping ratio may be greater than 0% and up to 10% (wt %).

The emission auxiliary layer may increase light-emission efficiency by compensating for an optical resonance distance of the wavelength of light emitted by an emission layer, and the electron-blocking layer may block the leakage of electrons from an emission layer to a hole transport region. Materials that may be included in the hole transport region may be included in the emission auxiliary layer and the electron-blocking layer.

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and/or a blue emission layer, in which the two or more layers contact each other or are separated from each other, to emit white light. In another embodiment, the emission layer may include two or more materials of a red light-emitting material, a green light-emitting material, and/or a blue light-emitting material, in which the two or more materials are mixed with each other in a single layer, to emit white light.

The emission layer may include a host and a dopant. The host may include the first host and the second host. The dopant included in the emission layer may include the aforementioned dopant. For example, the dopant may include a phosphorescent dopant.

In the emission layer, an amount of the dopant may be about 0.01 part by weight to about 15 parts by weight, based on 100 parts by weight of the total hosts.

In one or more embodiments, the emission layer may include a delayed fluorescence material. The delayed fluorescence material may act as a host or a dopant in the emission layer.

A thickness of the emission layer may be about 100 Å to about 1,000 Å, for example, about 200 Å to about 600 Å. For example, the interlayer may include the emission layer in the number of m, and when m is 2 or more, a thickness of each of the emission layers may be about 50 Å to about 500 Å. When the thickness of the emission layer is within the ranges above, excellent luminescence characteristics may be obtained without a substantial increase in driving voltage.

### Host in Emission Layer

The host may include the first host and the second host.

In one or more embodiments, the first host and the second host may each independently include a compound represented by Formula 301:

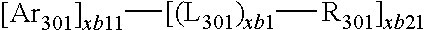

wherein, in Formula 301,
Ar₃₀₁ and L₃₀₁ may each independently be a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₇-C₆₀ alkyl aryl group that is unsubstituted or substituted with at least one R₁₀ₐ, a C₇-C₆₀ aryl alkyl group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, a C₁-C₆₀ alkyl heteroaryl group that is unsubstituted or substituted with at least one R₁₀ₐ, a C₂-C₆₀ heteroaryl alkyl group that is unsubstituted or substituted with at least one R₁₀ₐ, a C₁-C₆₀ heteroaryloxy group that is unsubstituted or substituted with at least one R₁₀ₐ, a C₁-C₆₀ arylthio group that is unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)(Q₃₀₁), —S(═O)₂(Q₃₀₁), —P(Q₃₀₁)(Q₃₀₂), or —P(═O)(Q₃₀₁)(Q₃₀₂),
xb21 may be an integer from 1 to 5, and
Q₃₀₁ to Q₃₀₃ may each be as described herein in connection with Q₁.

In one or more embodiments, when xb11 in Formula 301 is 2 or more, two or more of Ar₃₀₁ may be linked to each other via a single bond.

In another embodiment, the first host and the second host may each independently be a compound represented by Formula 301-1, a compound represented by Formula 301-2, or a combination thereof, but embodiments are not limited thereto:

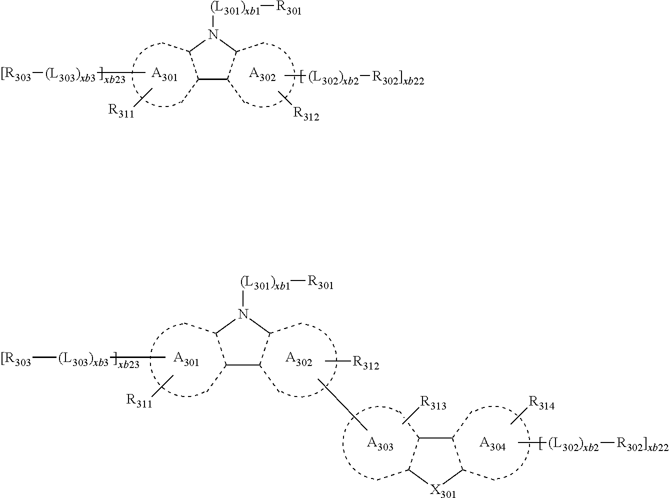

wherein, in Formulae 301-1 and 301-2,
ring A₃₀₁ to ring A₃₀₄ may each independently be a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
X₃₀₁ may be —O—, —S—, —N—[(L₃₀₄)_{xba}-R₃₀₄]—, —C(R₃₀₄)(R₃₀₅)—, or —Si(R₃₀₄)(R₃₀₅)—,
xb22 and xb23 may each independently be 0, 1, or 2,
L₃₀₁, xb1, and R₃₀₁ may each be as described herein,
L₃₀₂ to L₃₀₄ may each independently be as described herein in connection with L₃₀₁,
xb2 to xb4 may each independently be as described herein in connection with xb1, and
R₃₀₂ to R₃₀₅ and R₃₁₁ to R₃₁₄ may each be as described herein in connection with R₃₀₁.

In another embodiment, the first host and the second host may each independently be an alkaline earth metal complex, a post-transition metal complex, or a combination thereof. In one or more embodiments, the first host and the second host may each independently include a Be complex (for example, Compound H55 or the like), a Mg complex, a Zn complex, or a combination thereof.

In another embodiment, the first host and the second host may include one of Compounds H1 to H130; 9,10-di(2-naphthyl)anthracene (ADN); 2-methyl-9,10-bis(naphthalen-2-yl)anthracene (MADN); 9,10-di-(2-naphthyl)-2-t-butyl-anthracene (TBADN); 4,4′-bis(N-carbazolyl)-1,1′-biphenyl (CBP); 1,3-di-9-carbazolylbenzene (mCP); 1,3,5-tri(carbazol-9-yl)benzene (TCP); or a combination thereof, but embodiments are not limited thereto:

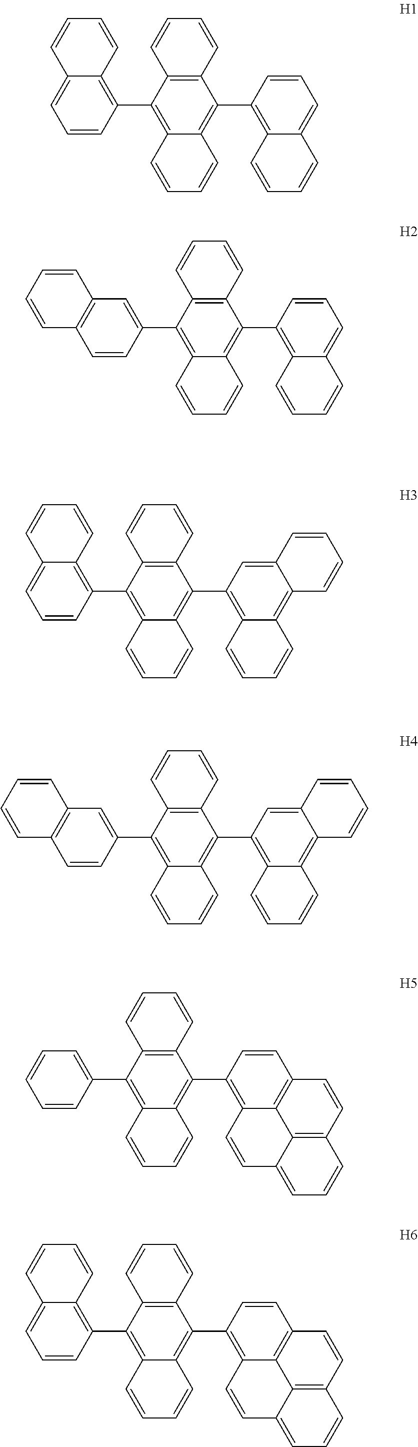

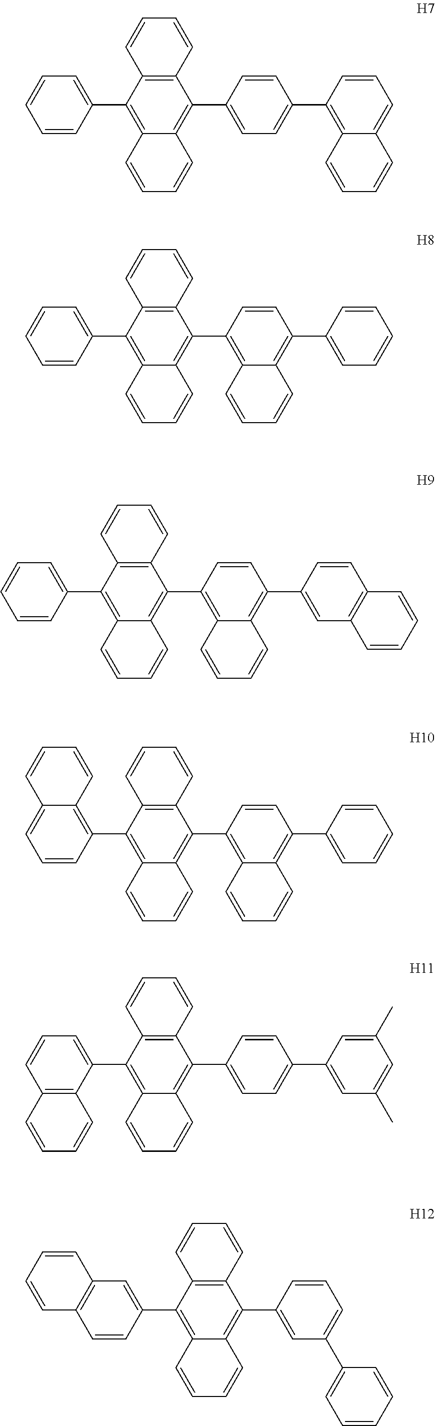

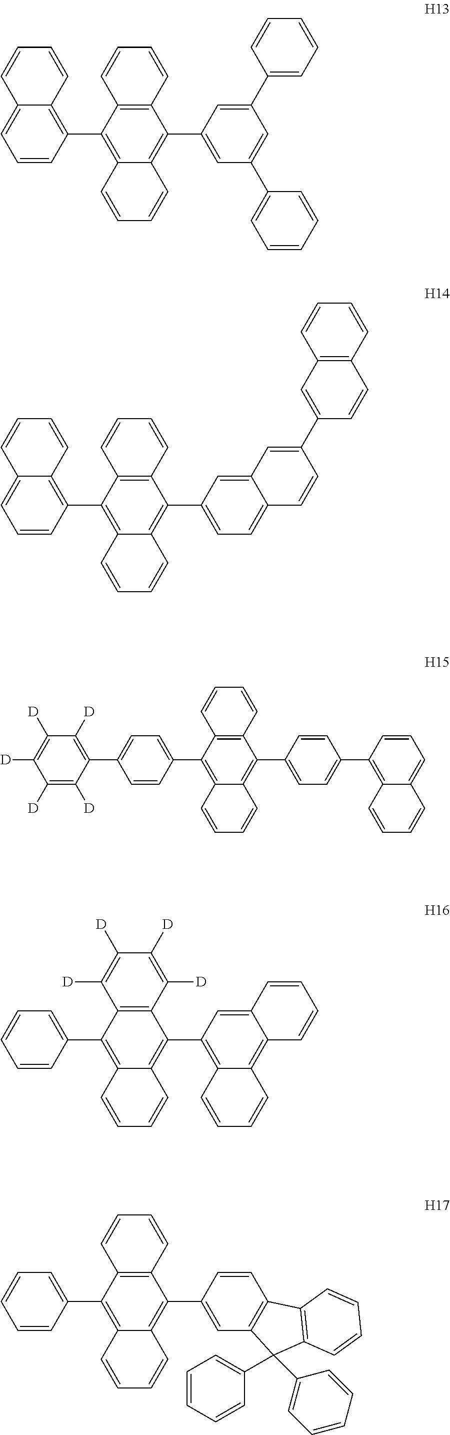

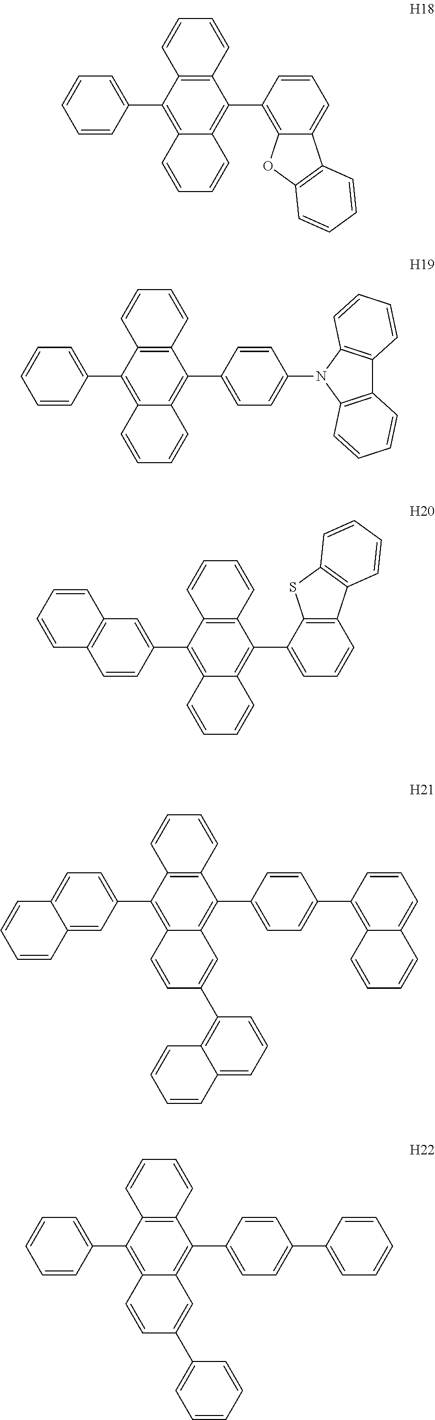

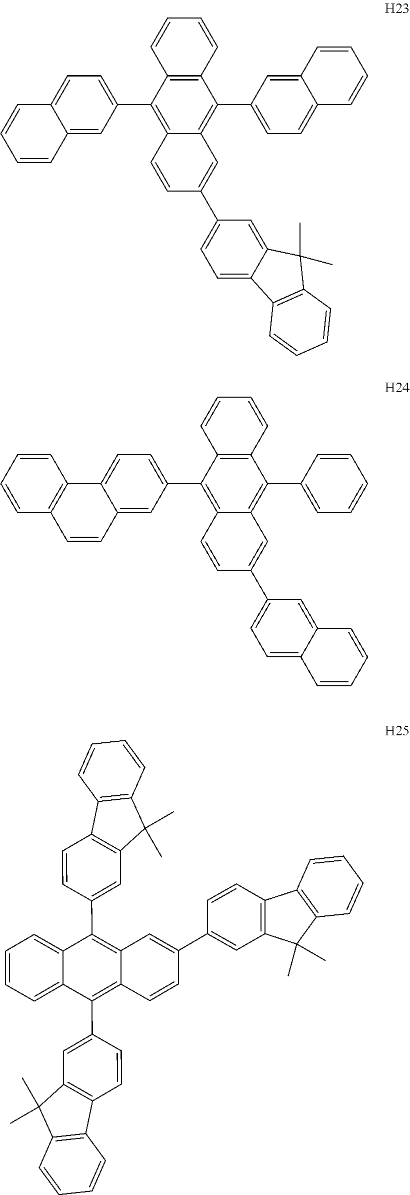

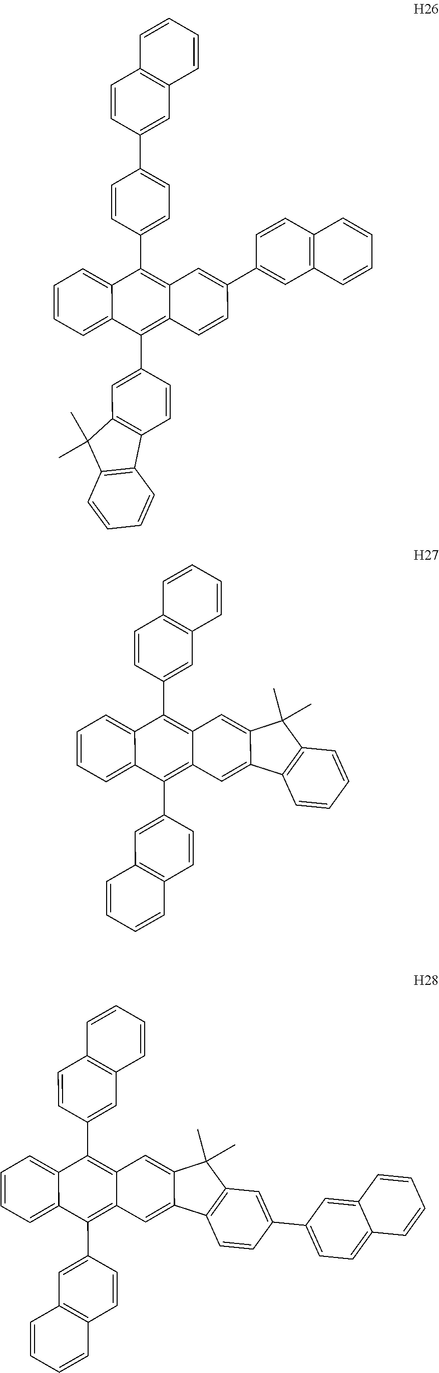

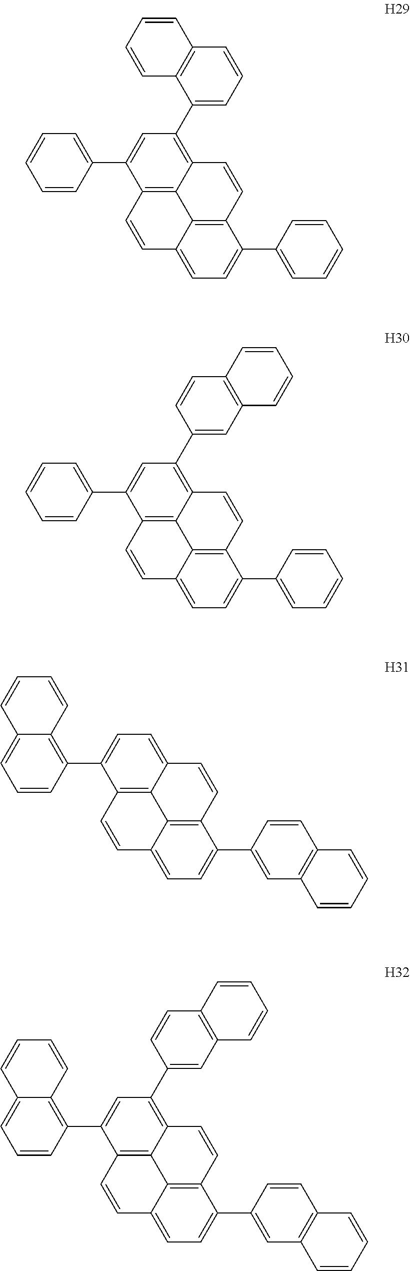

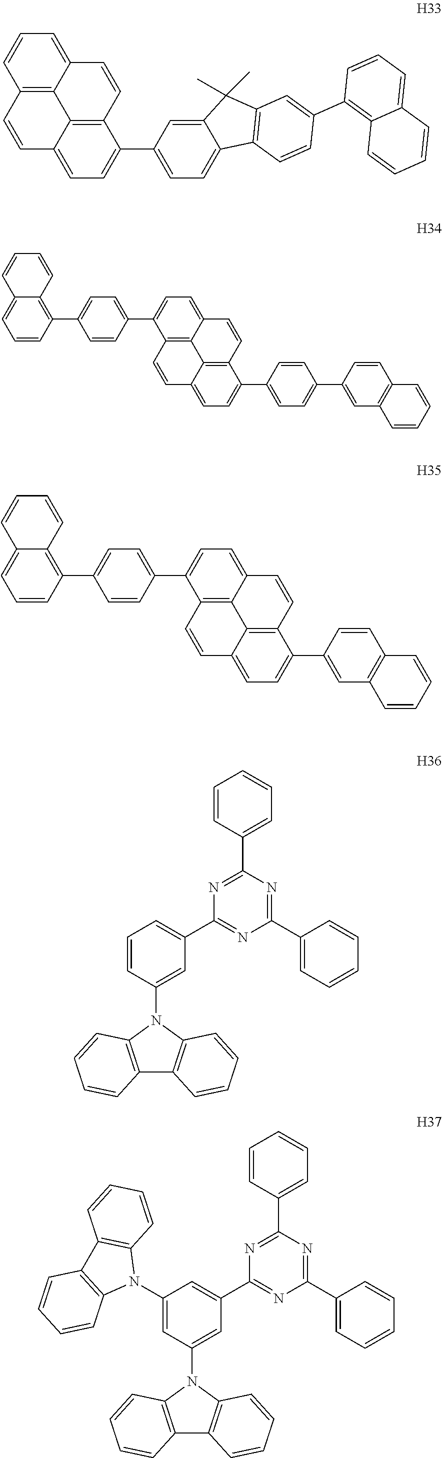

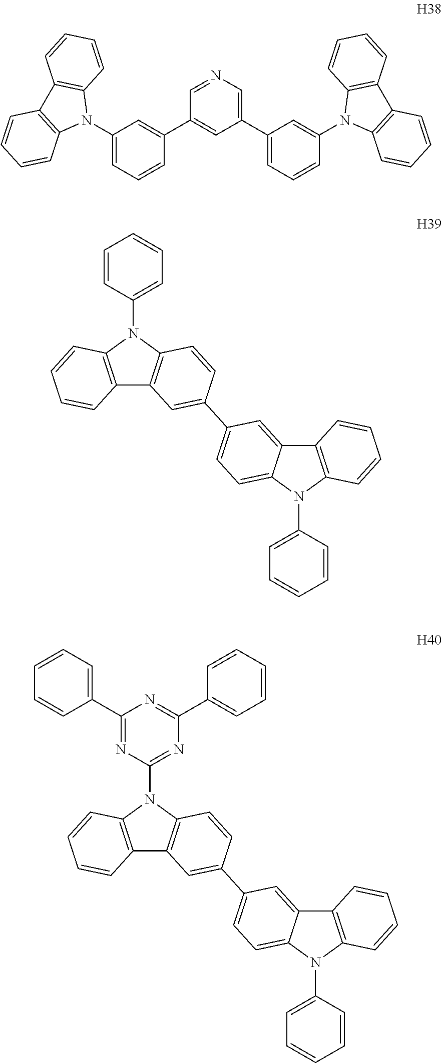 [SMILES: c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1]

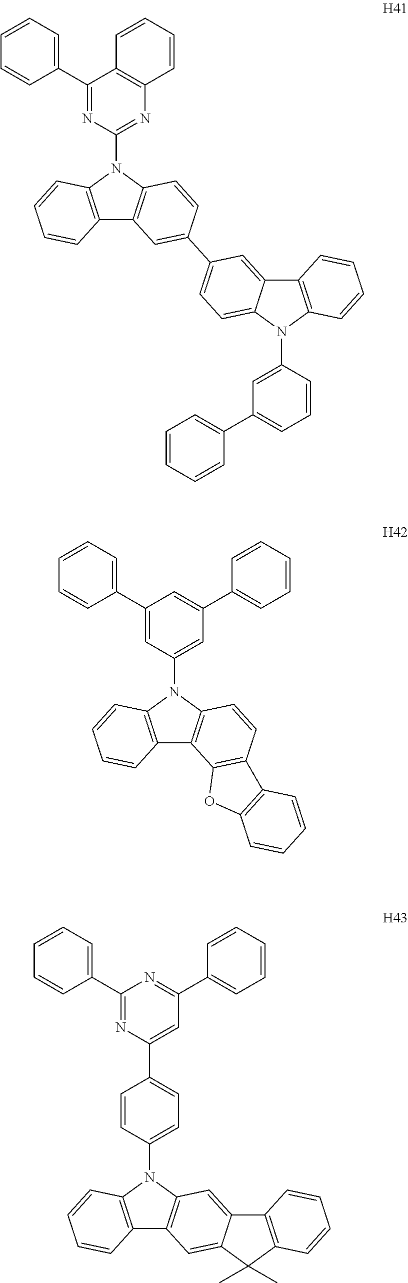 [SMILES: c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1 ; CC1(C)c2ccccc2-c2cc3c(cc21)c1ccccc1n3-c1ccc(-c2cc(-c3ccccc3)nc(-c3ccccc3)n2)cc1]

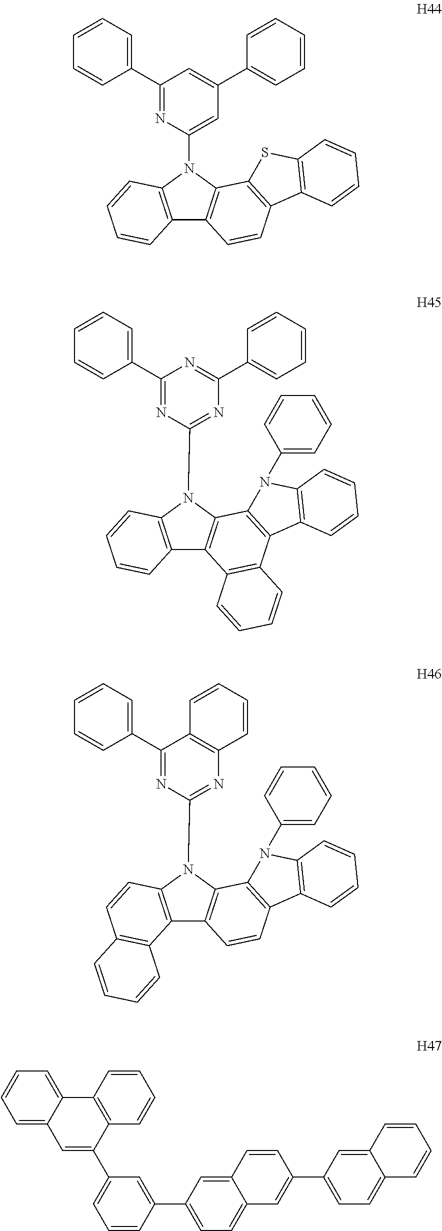

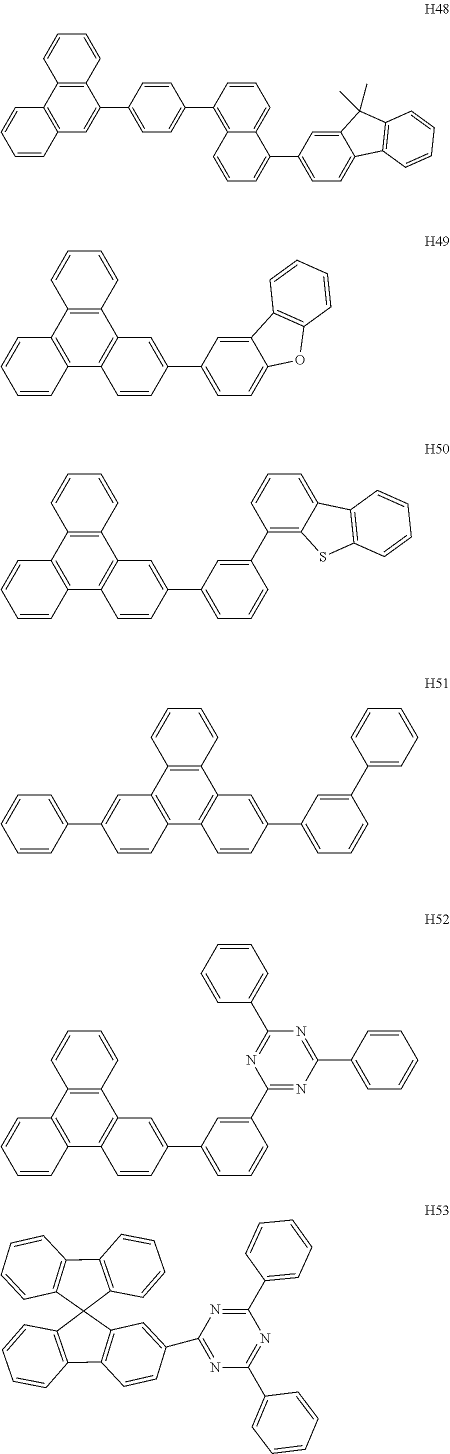

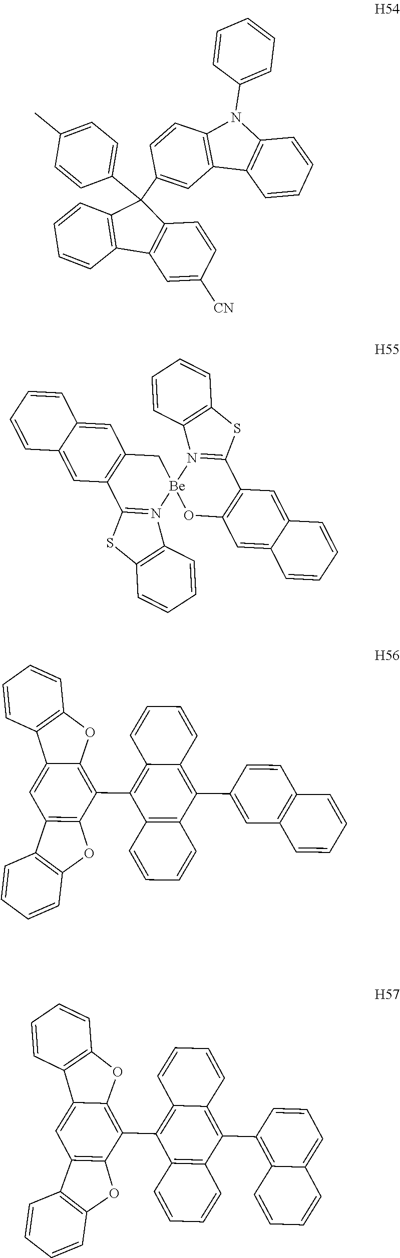

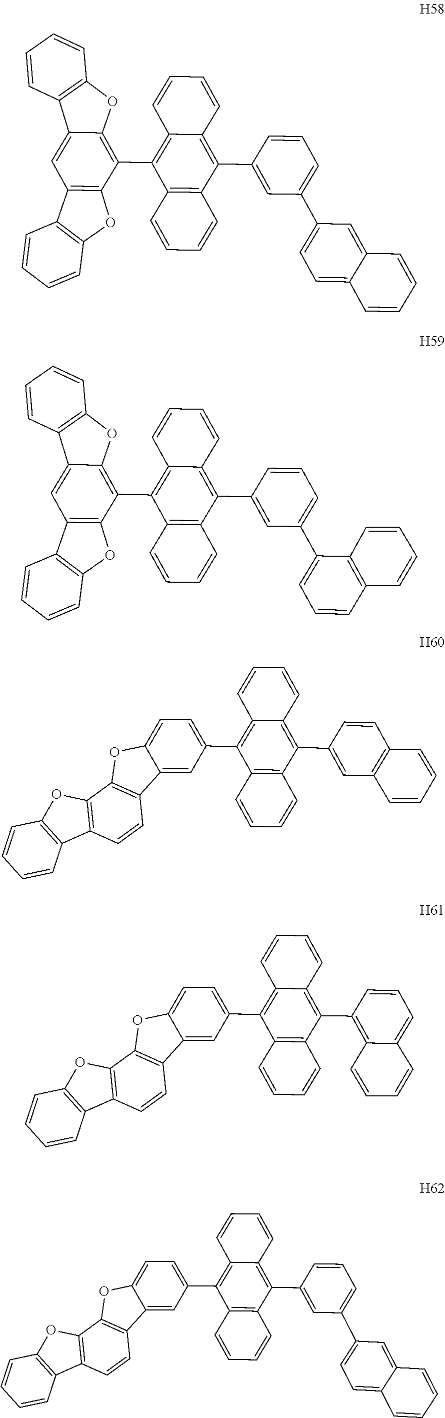 [SMILES: c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1]

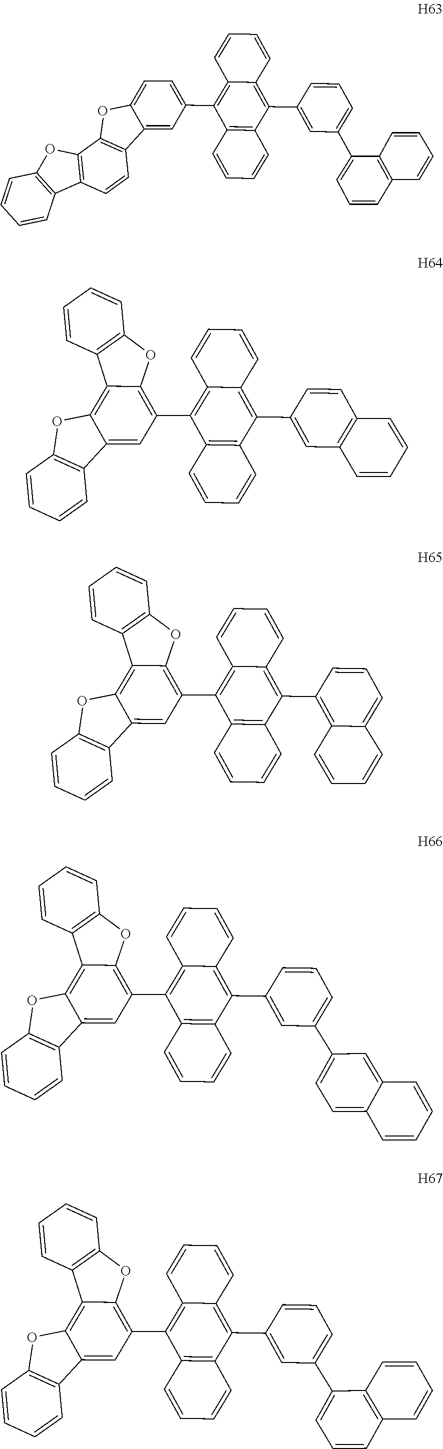 [SMILES: c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1 ; c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1]

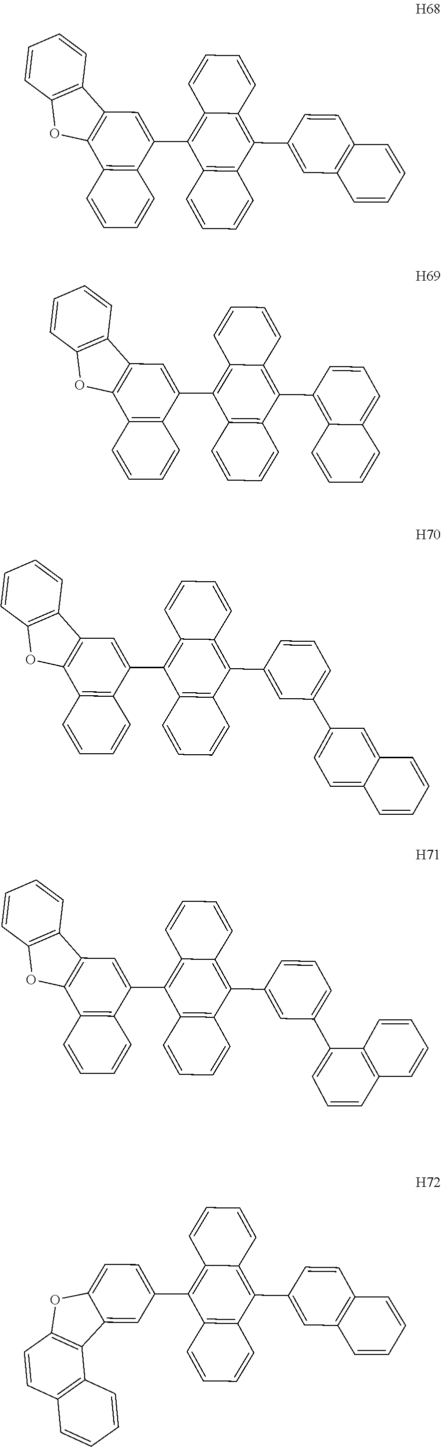 [SMILES: c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1]

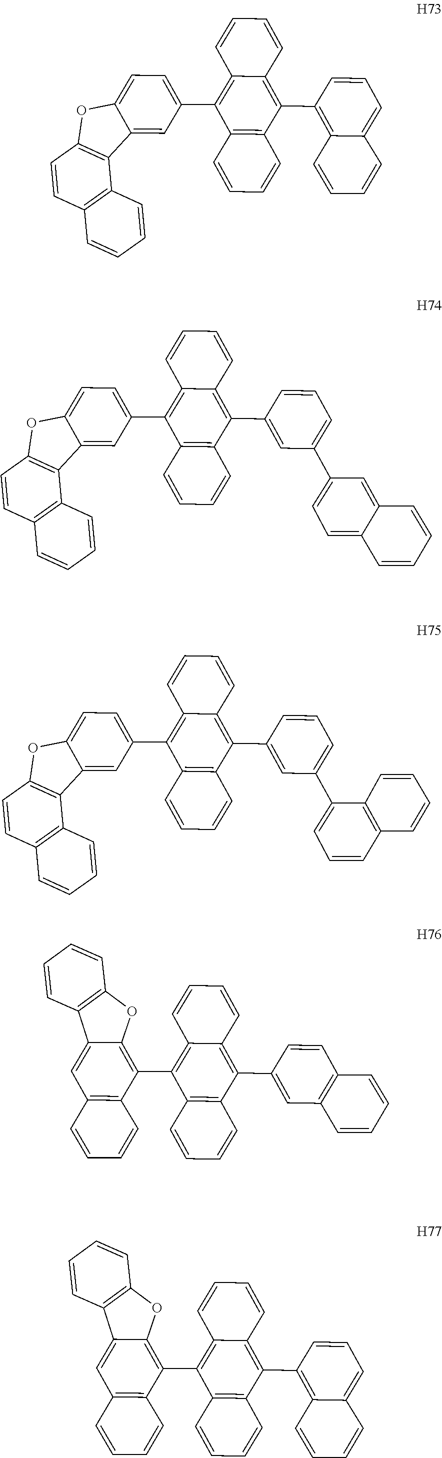 [SMILES: c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1 ; c1ccc2c(-c3c4ccccc4c(-c4c5ccccc5cc5c4oc4ccccc45)c4ccccc34)cccc2c1]

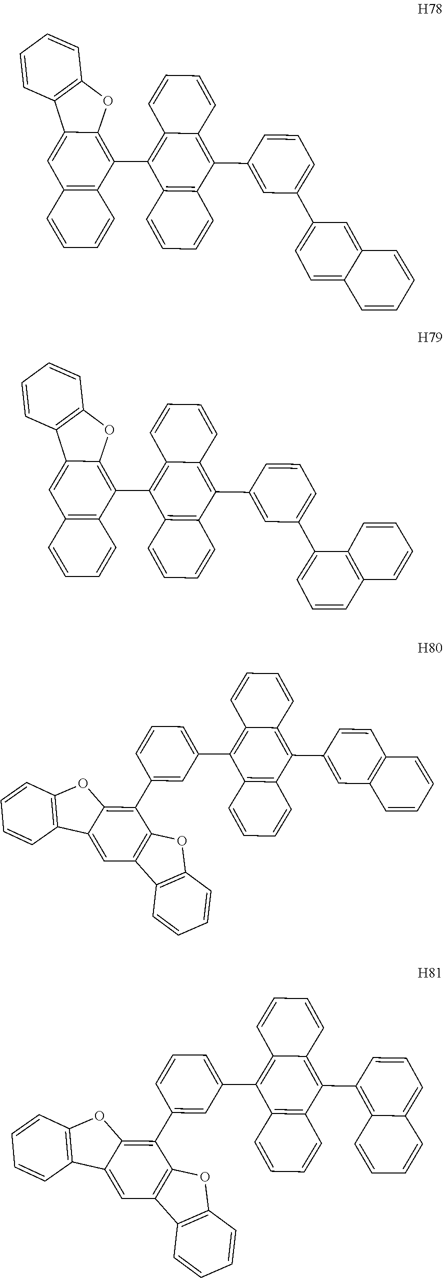 [SMILES: c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1]

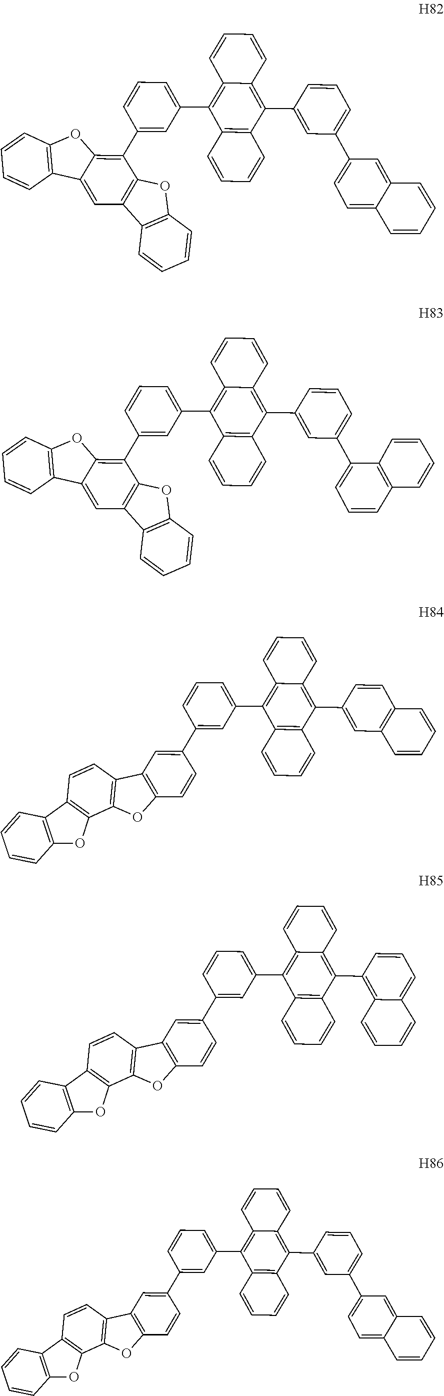 [SMILES: c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1]

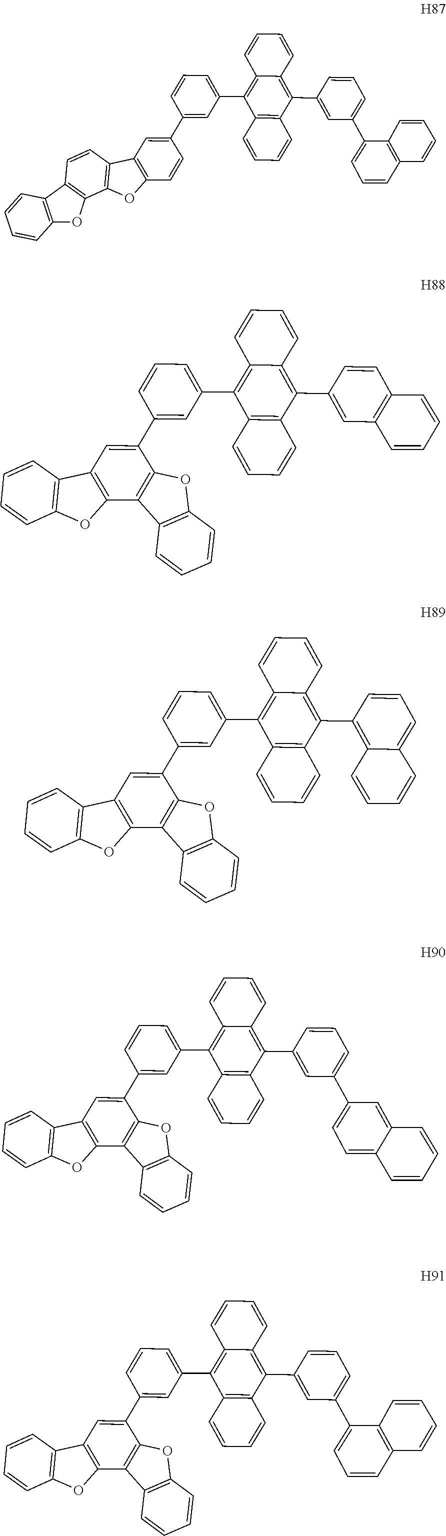 [SMILES: c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1]

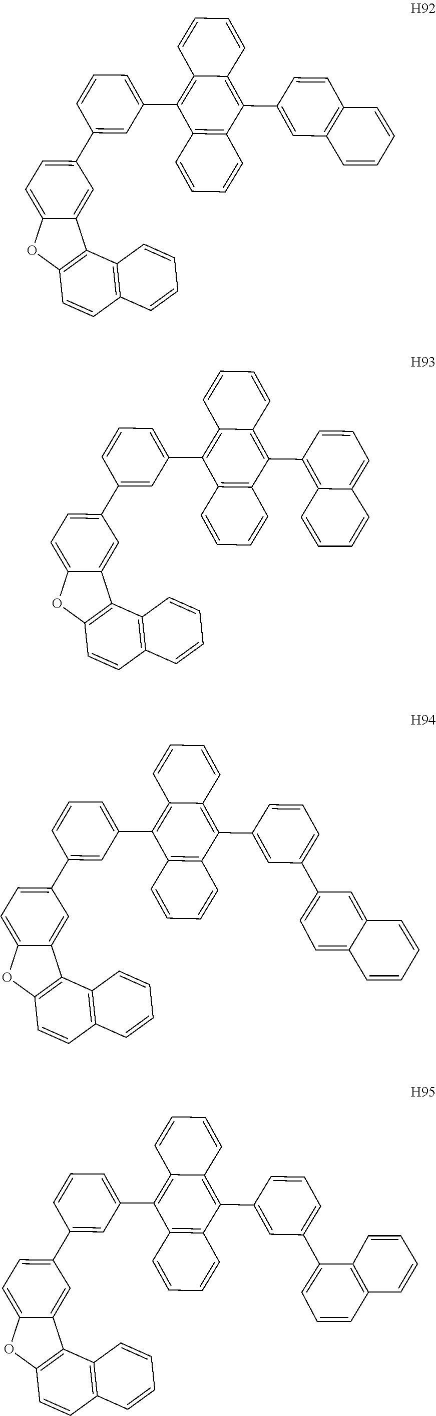 [SMILES: c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1]

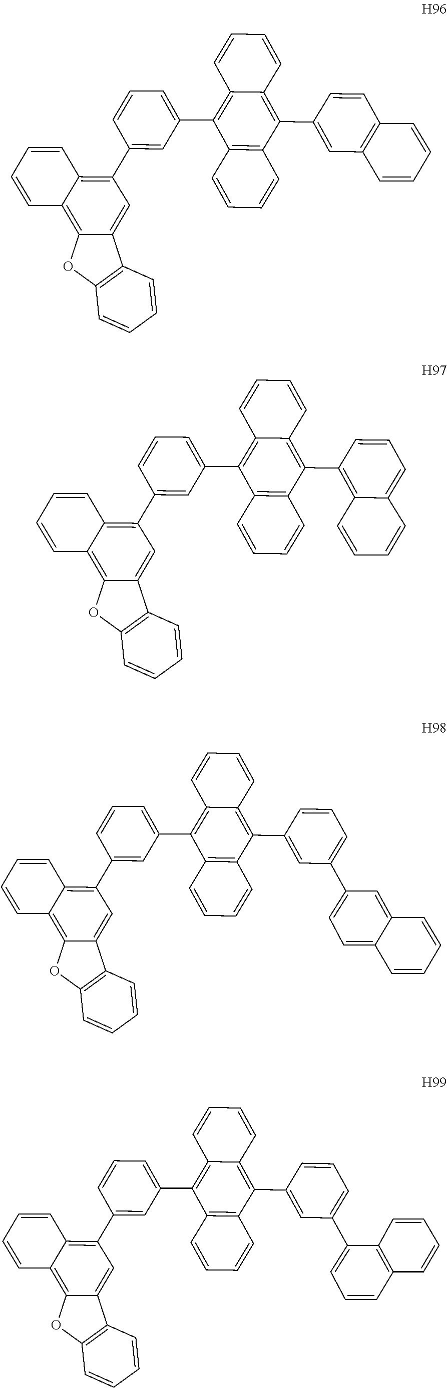 [SMILES: c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1]

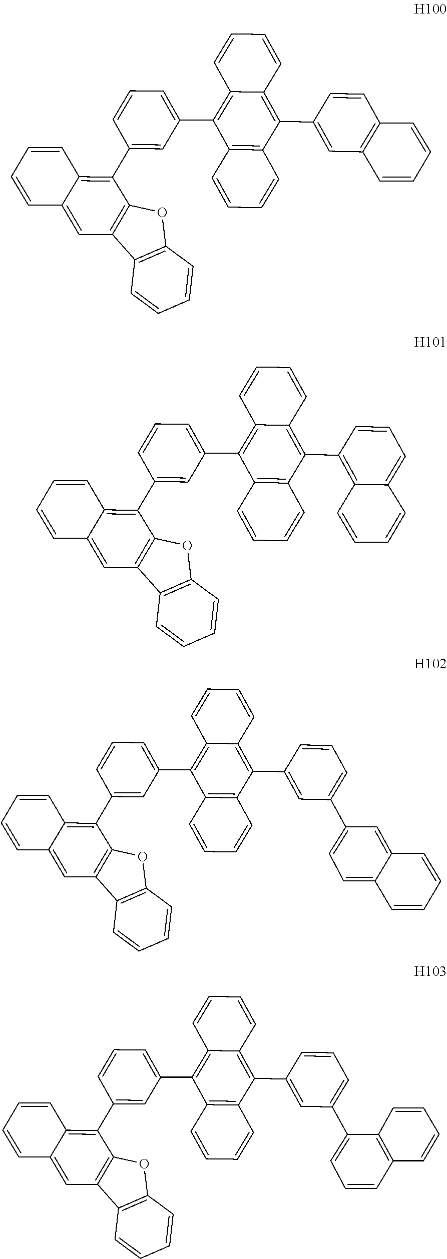 [SMILES: c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1]

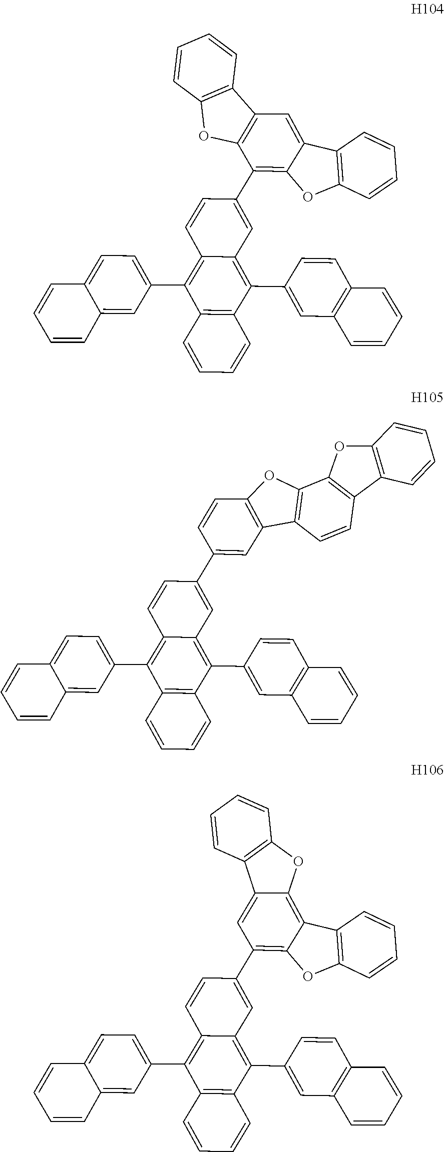 [SMILES: c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1]

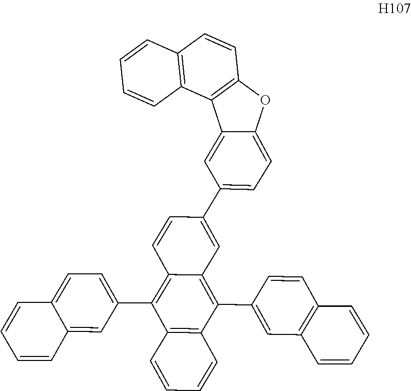 [SMILES: c1ccc2cc(-c3c4ccccc4c(-c4ccc5ccccc5c4)c4cc(-c5ccc6oc7ccc8ccccc8c7c6c5)ccc34)ccc2c1]

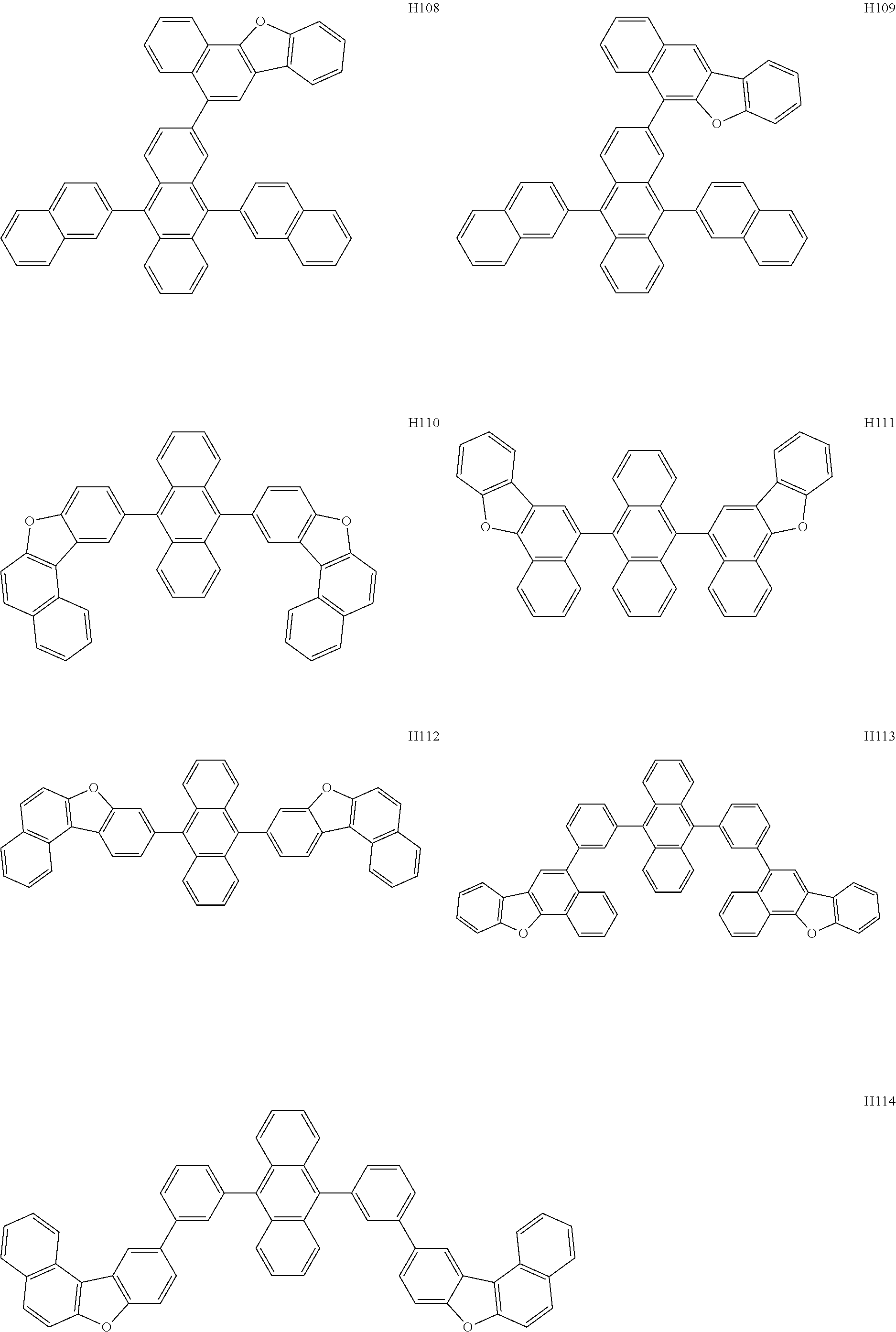

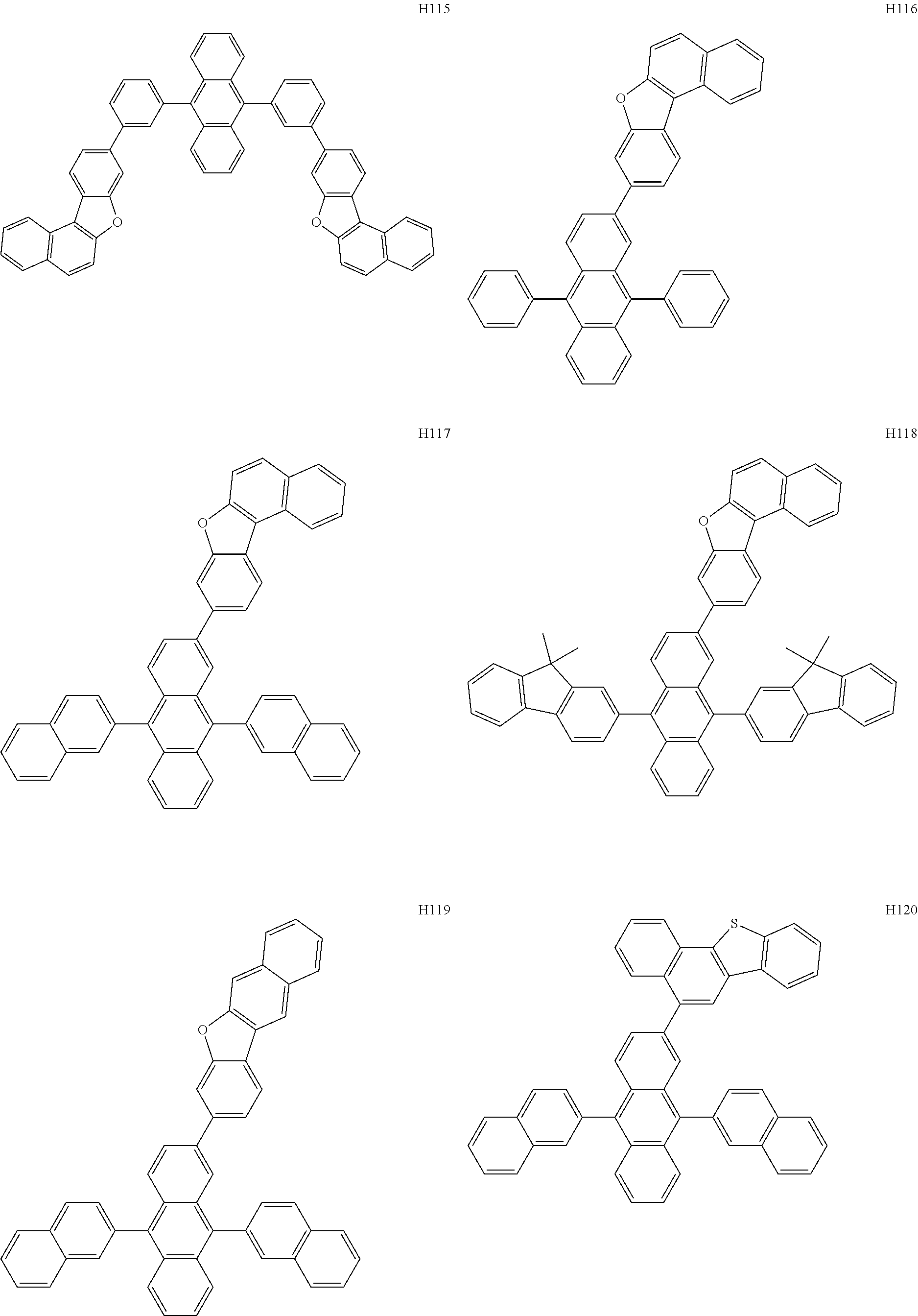

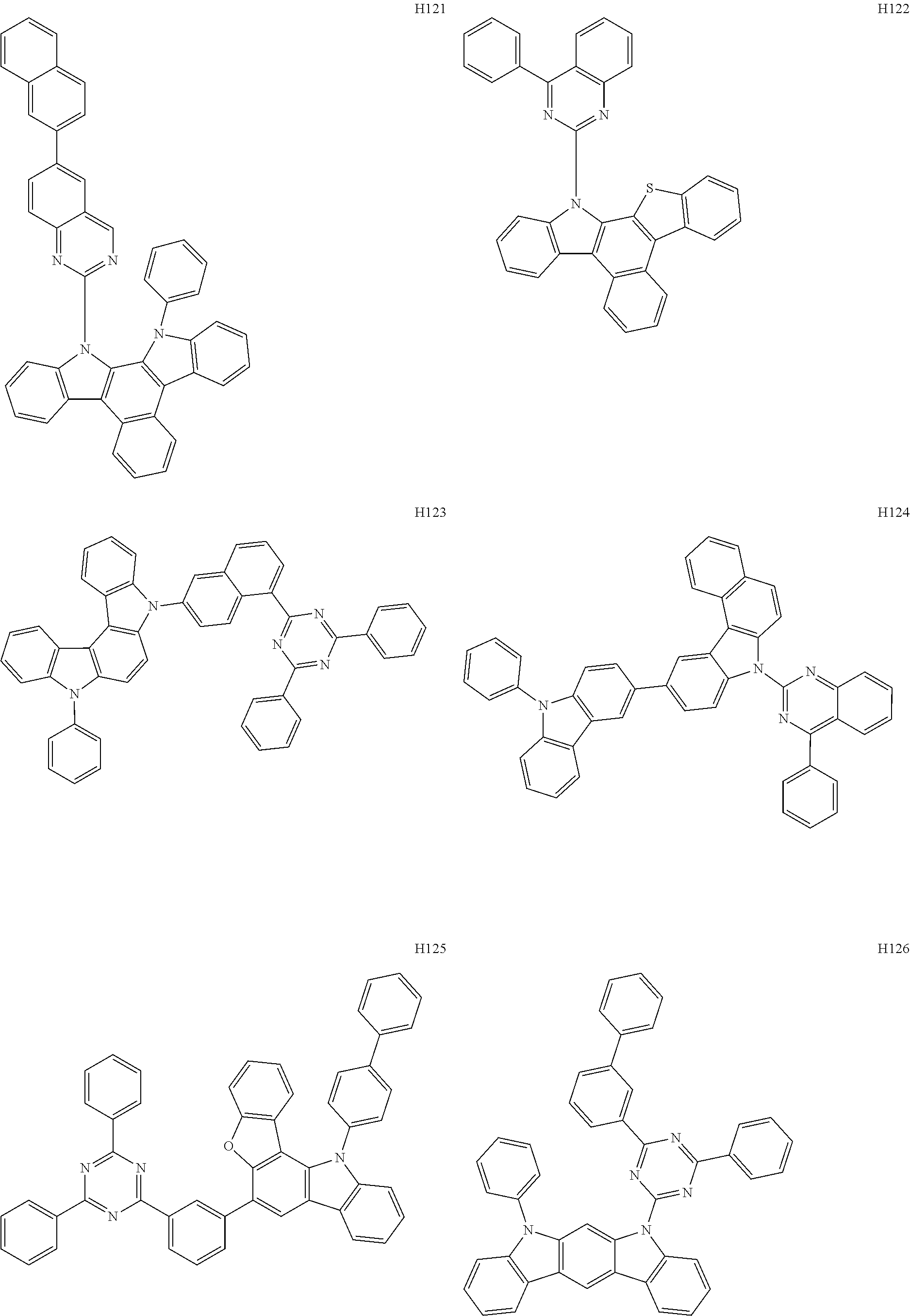

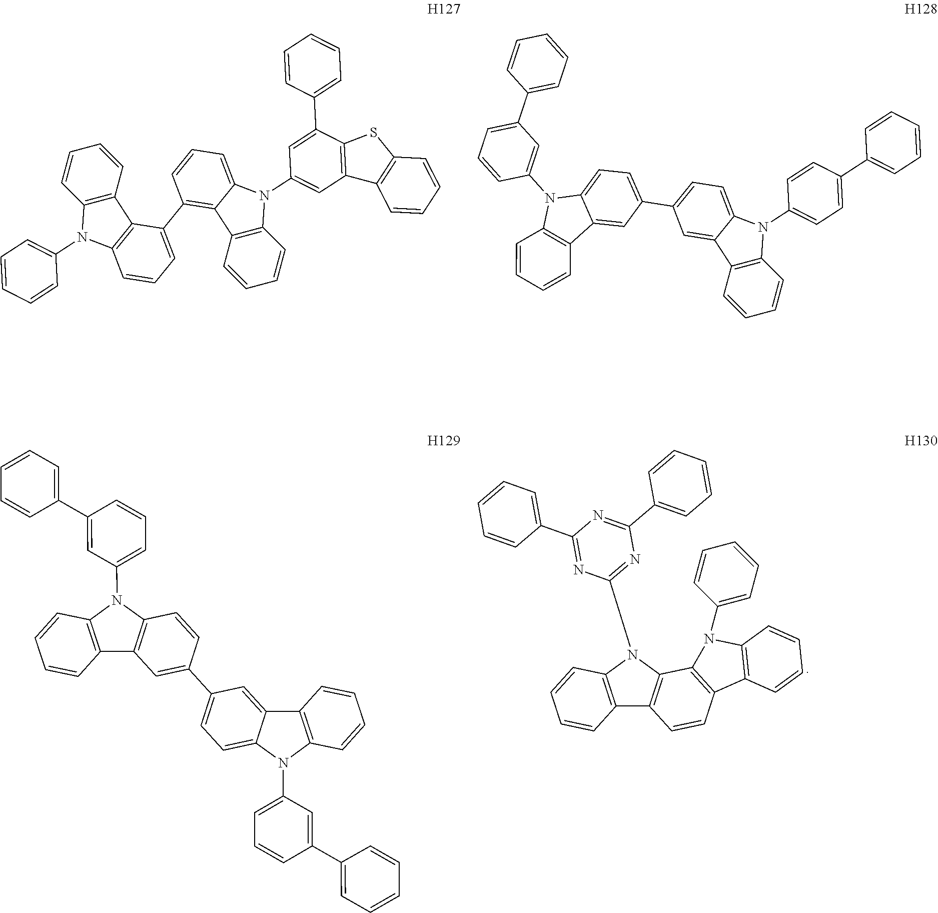 [SMILES: c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2nc(-c3ccccc3)nc(-n3c4ccccc4c4ccc5c6ccccc6n(-c6ccccc6)c5c43)n2)cc1]

### Phosphorescent Dopant

The phosphorescent dopant may include the aforementioned dopant.

In one or more embodiments, an amount of the dopant may be about 1.0 wt % to about 30 wt % (based on 100 parts by weight of total compounds in the emission layer). When the amount of the dopant is within this range, the light-emitting device may have excellent efficiency and lifespan.

The dopant may include, for example, one of Compounds PD26 to PD40, but embodiments are not limited thereto:

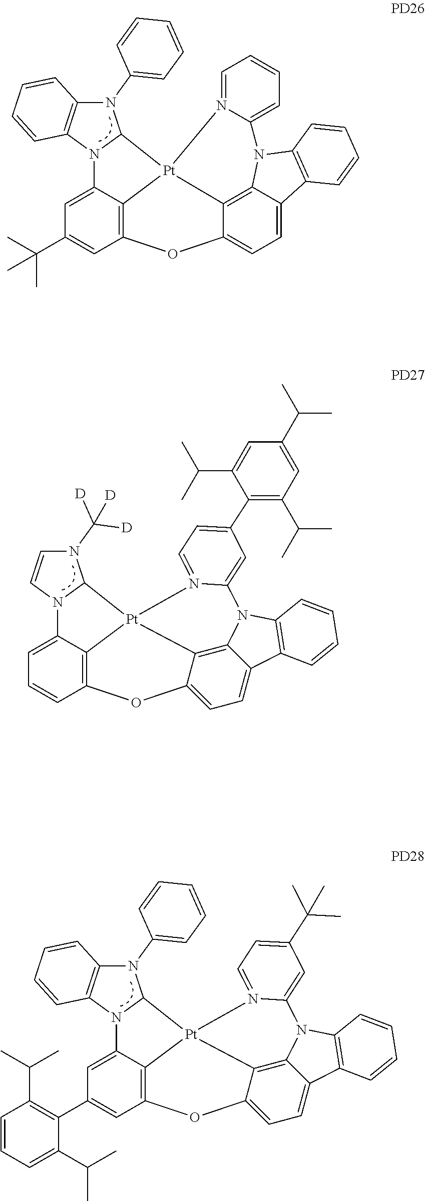

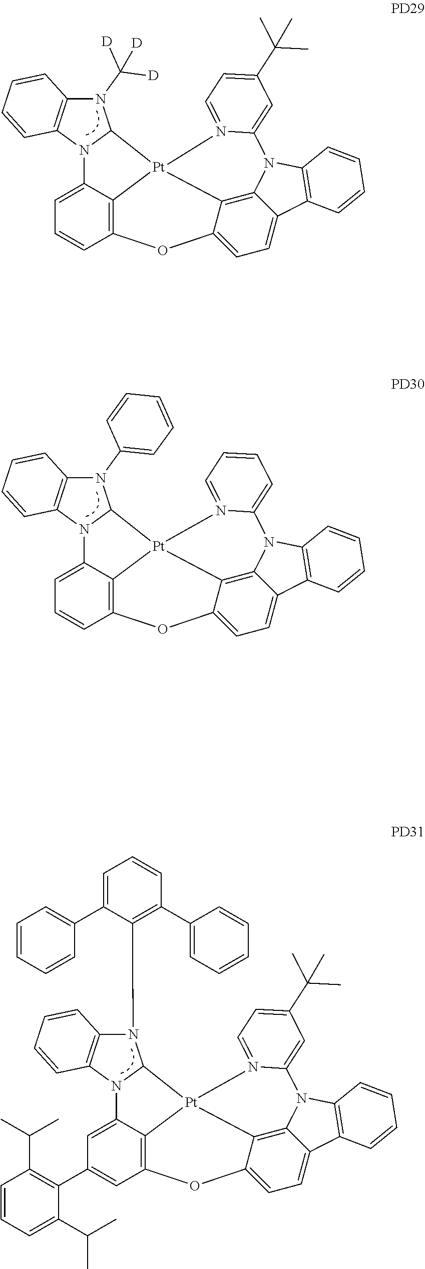

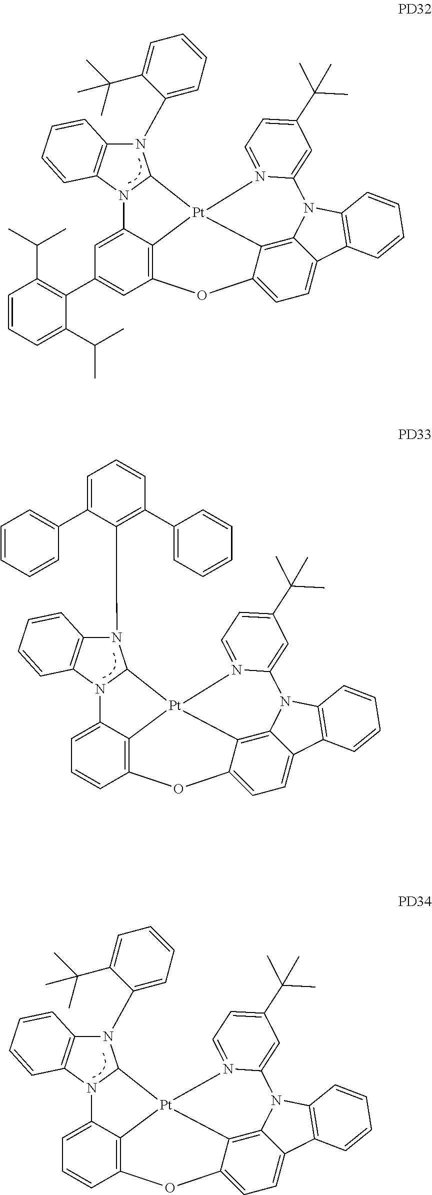

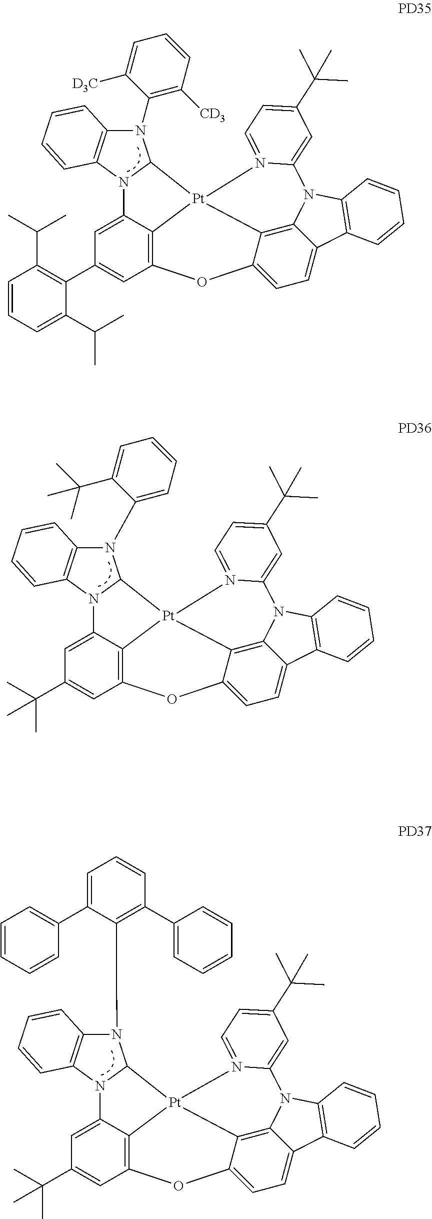

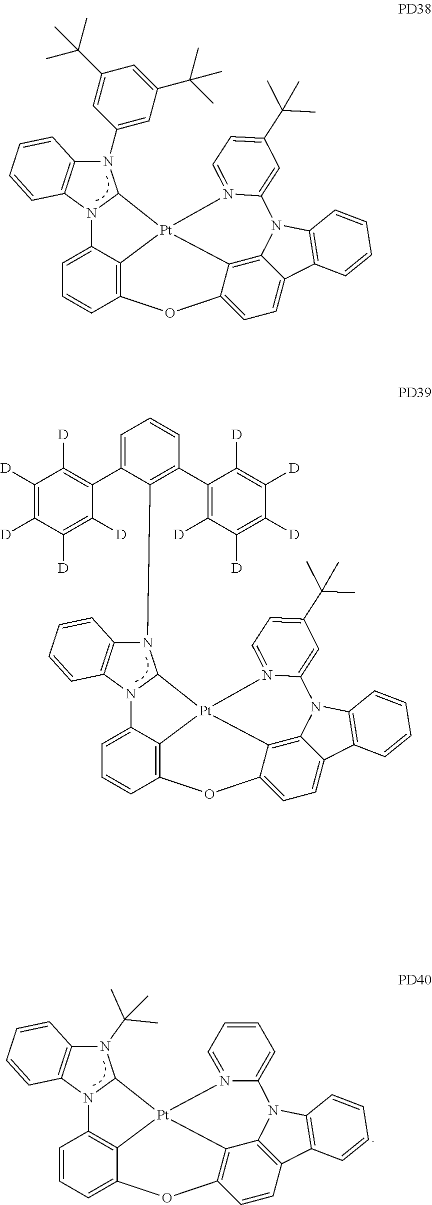

### Electron Transport Region in Interlayer 130

The electron transport region may have: i) a single-layer structure consisting of a single layer consisting of a single material, ii) a single-layer structure consisting of a single layer consisting of multiple materials that are different from each other, or iii) a multi-layer structure including multiple layers including multiple materials that are different from each other.

The electron transport region may include an electron injection layer, a hole-blocking layer, an electron transport layer, or a combination thereof.

In one or more embodiments, the electron transport region may be arranged between the emission layer and the charge generation layer, and may include a hole-blocking layer, an electron transport layer, an electron injection layer, or a combination thereof. For example, the electron transport region may have an electron transport auxiliary layer/electron transport layer structure, an electron transport auxiliary layer/electron transport layer/electron injection layer structure, or the like, wherein constituent layers of each structure are sequentially stacked in the stated order from the emission layer.

In one or more embodiments, the electron transport region (e.g., the hole-blocking layer or the electron transport layer in the electron transport region) may include a metal-free compound including at least one Tr electron-deficient nitrogen-containing C₁-C₆₀ cyclic group.

In one or more embodiments, the electron transport region may include a compound represented by Formula 601:

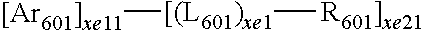

wherein, in Formula 601,
Ar₆₀₁ and L₆₀₁ may each independently be a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₆₀₁)(Q₆₀₂)(Q₆₀₃), —C(═O)(Q₆₀₁), —S(═O)(Q₆₀₁), —S(═O)₂(Q₆₀₁), —P(Q₆₀₁)(Q₆₀₂), or —P(═O)(Q₆₀₁)(Q₆₀₂),
Q₆₀₁ to Q₆₀₃ may each be as described herein in connection with Q₁,
xe21 may be 1, 2, 3, 4, or 5, and
at least one of Ar₆₀₁, L₆₀₁, and R₆₀₁ may each independently be a π electron-deficient nitrogen-containing C₁-C₆₀ cyclic group that is unsubstituted or substituted with at least one R₁₀ₐ.

In one or more embodiments, when xe11 in Formula 601 is 2 or more, two or more of Ar₆₀₁ may be linked together via a single bond.

In one or more embodiments, Ar₆₀₁ in Formula 601 may be a substituted or unsubstituted anthracene group.

In one or more embodiments, the electron transport region may include a compound represented by Formula 601-1:

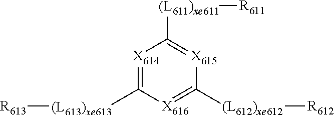

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ may each be as described herein in connection with L₆₀₁,
xe611 to xe613 may each be as described herein in connection with xe1,
R₆₁₁ to R₆₁₃ may each be as described herein in connection with R₆₀₁, and
R₆₁₄ to R₆₁₆ may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group that is unsubstituted or substituted with at least one R₁₀ₐ, a C₁-C₂₀ alkoxy group that is unsubstituted or substituted with at least one R₁₀ₐ, a C₁-C₂₀ alkylthio group that is unsubstituted or substituted with at least one R₁₀ₐ, a C₃-C₆₀ carbocyclic group that is unsubstituted or substituted with at least one R₁₀ₐ, or a C₁-C₆₀ heterocyclic group that is unsubstituted or substituted with at least one R₁₀ₐ.

In one or more embodiments, xe1 and xe611 to xe613 in Formulae 601 and 601-1 may each independently be 0, 1, or 2.

In another embodiment, the electron transport region may include one of Compounds ET1 to ET46; 2,9-dimethyl-4,7-diphenyl-1,10-phenanthroline (BCP); 4,7-diphenyl-1,10-phenanthroline (Bphen); tris(8-hydroxy-quinolinato)aluminum (Alq₃); bis(2-methyl-8-quinolinolato-N1,O8)-(1,1′-biphenyl-4-olato)aluminum (BAIq); 3-(4-biphenylyl)-4-phenyl-5-tert-butylphenyl-1,2,4-triazole (TAZ); 4-(naphthalen-1-yl)-3,5-diphenyl-4H-1,2,4-triazole (NTAZ); or a combination thereof, but embodiments are not limited thereto:

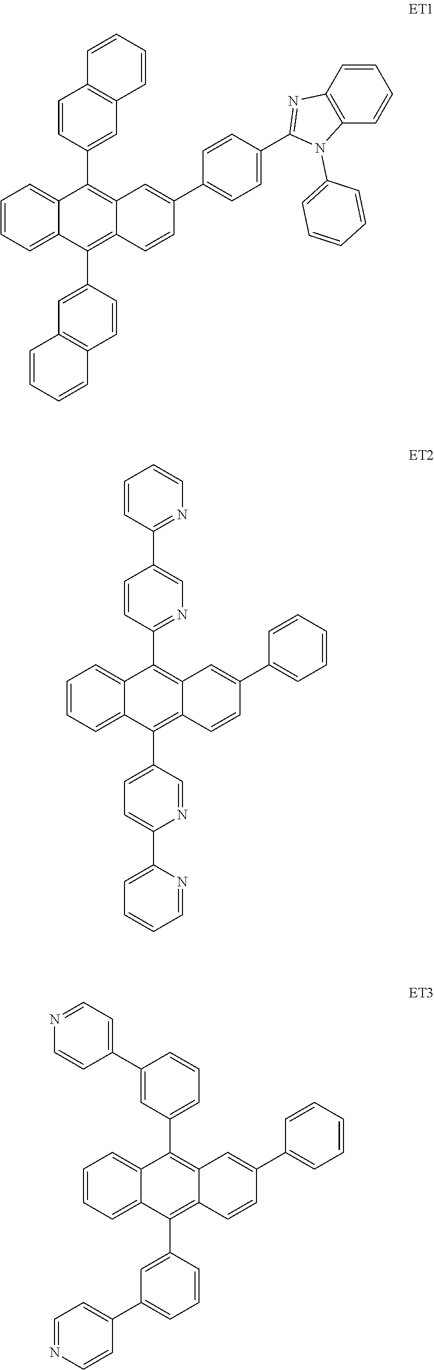 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)cn4)c3c2)cc1 ; c1ccc(-c2ccc3c(-c4cccc(-c5ccncc5)c4)c4ccccc4c(-c4cccc(-c5ccncc5)c4)c3c2)cc1]

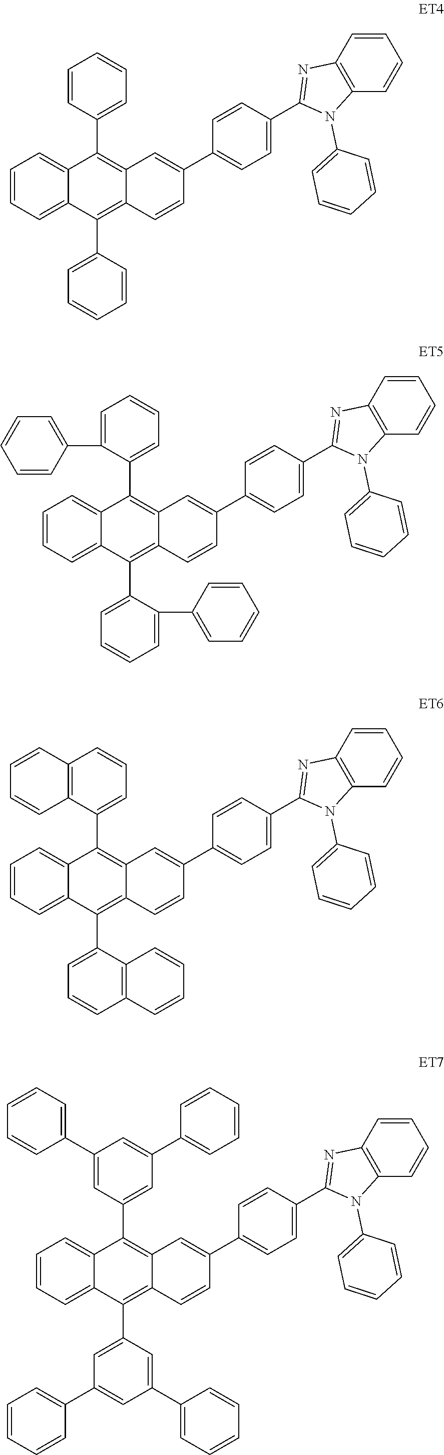 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

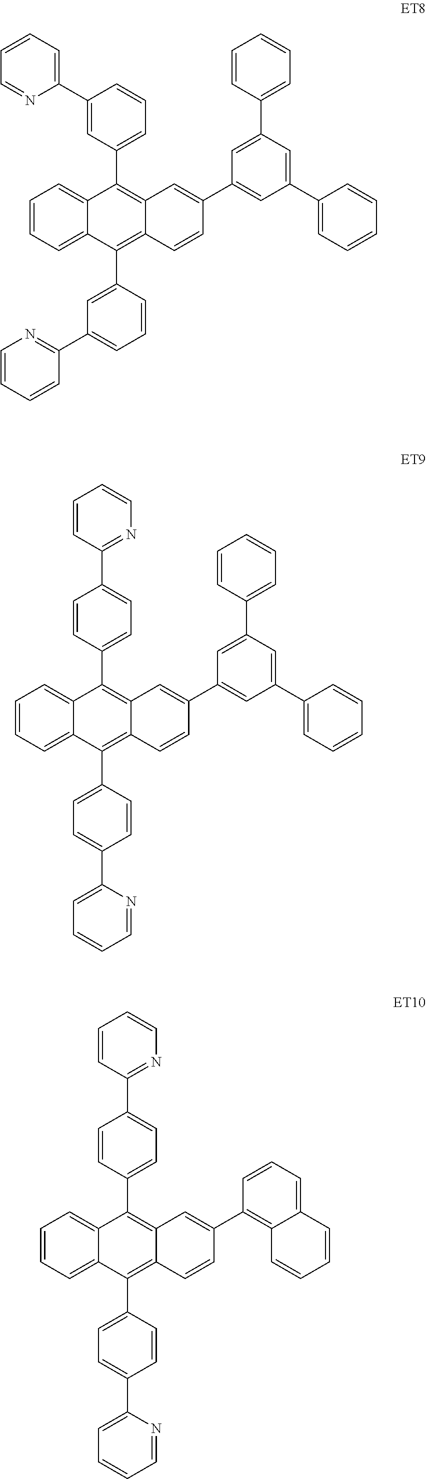 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

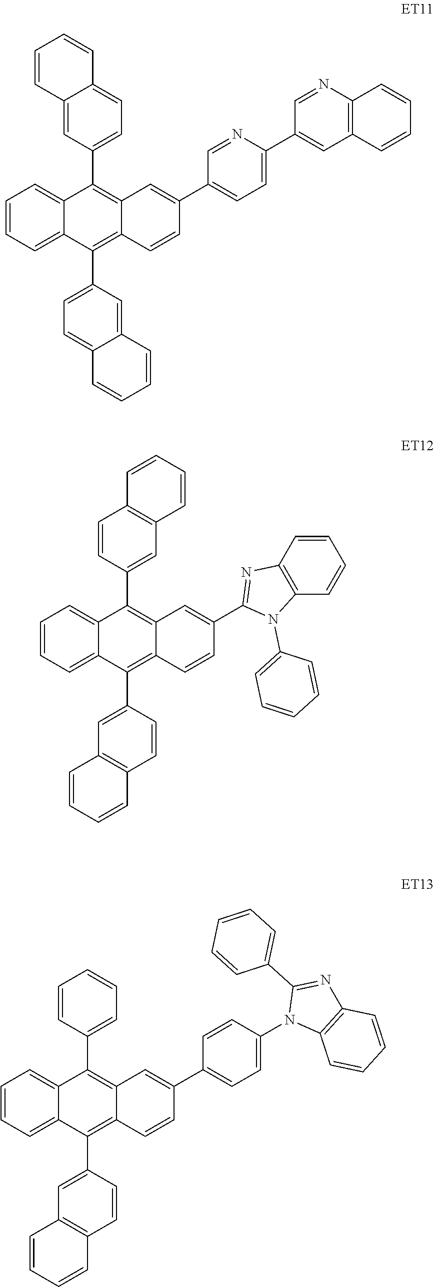 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

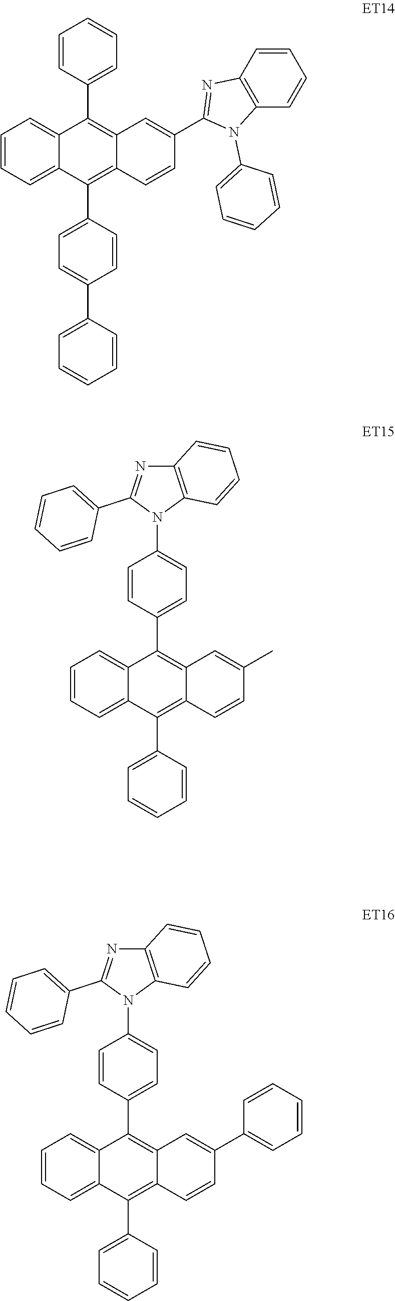 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

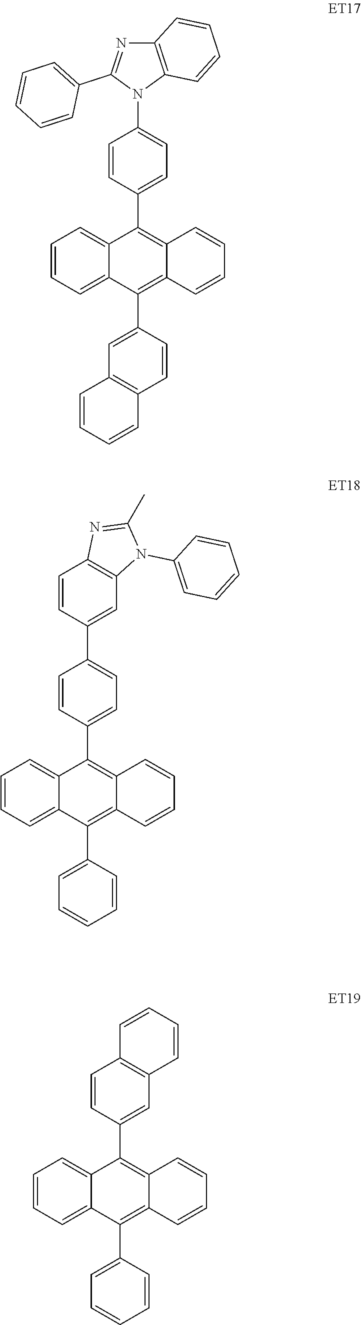

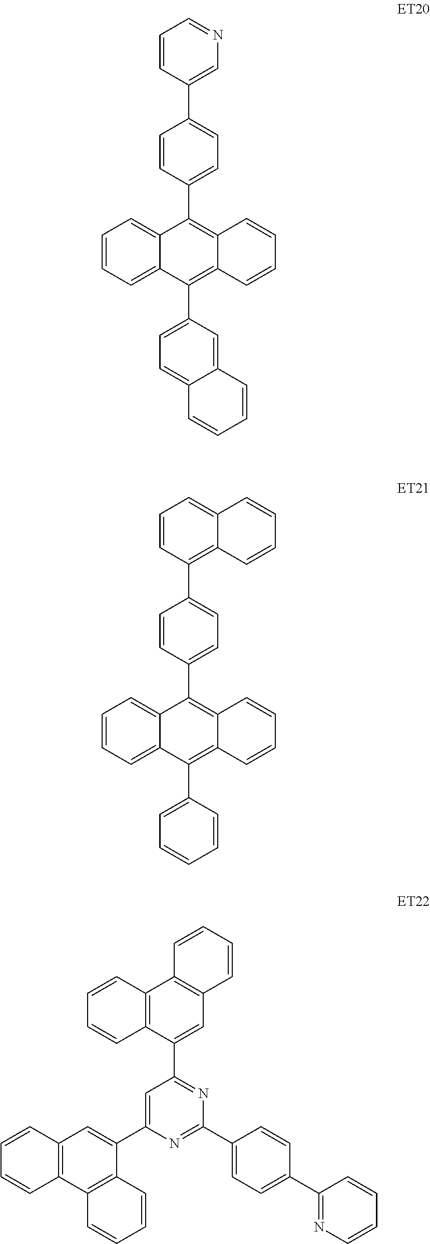

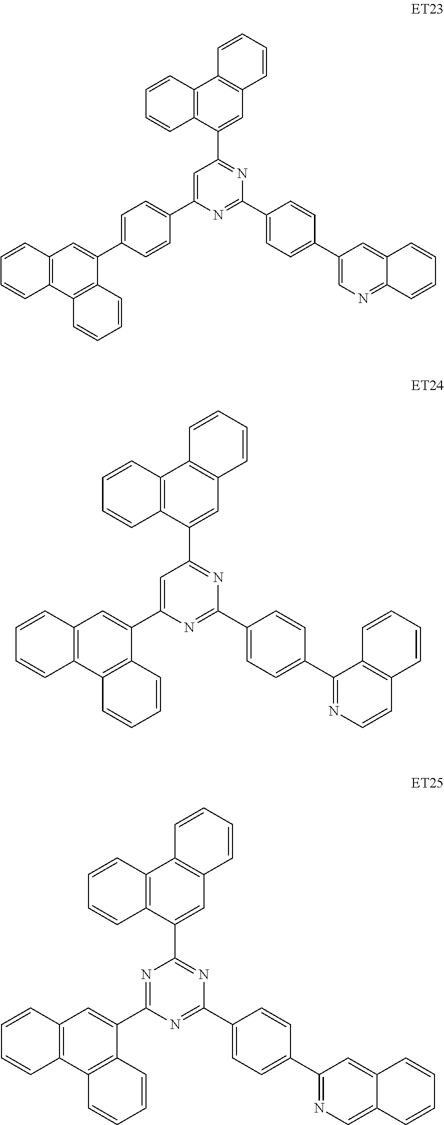 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2cc(-c3ccc(-c4nc(-c5cc6ccccc6c6ccccc56)nc(-c5cc6ccccc6c6ccccc56)n4)cc3)ncc2c1]

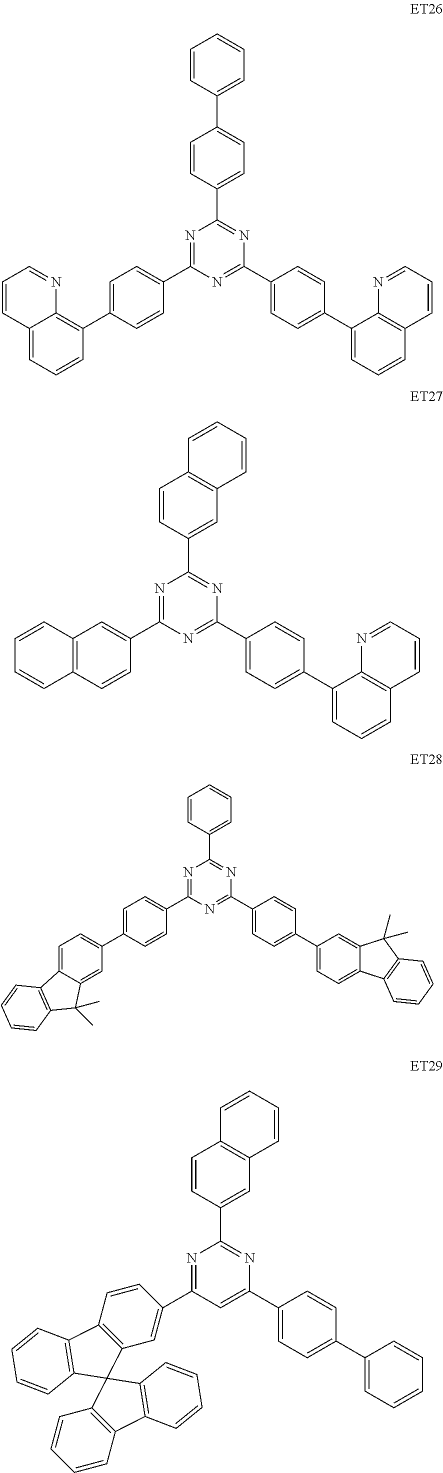 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

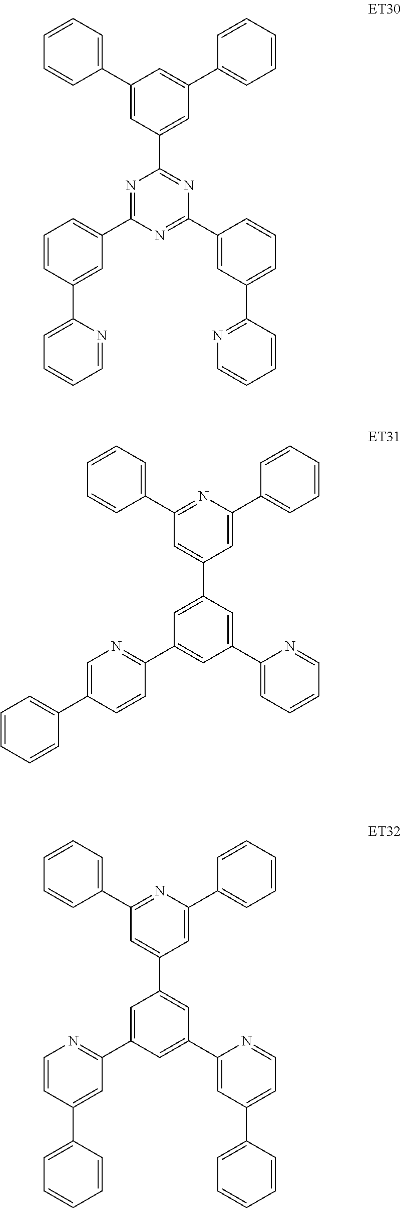 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

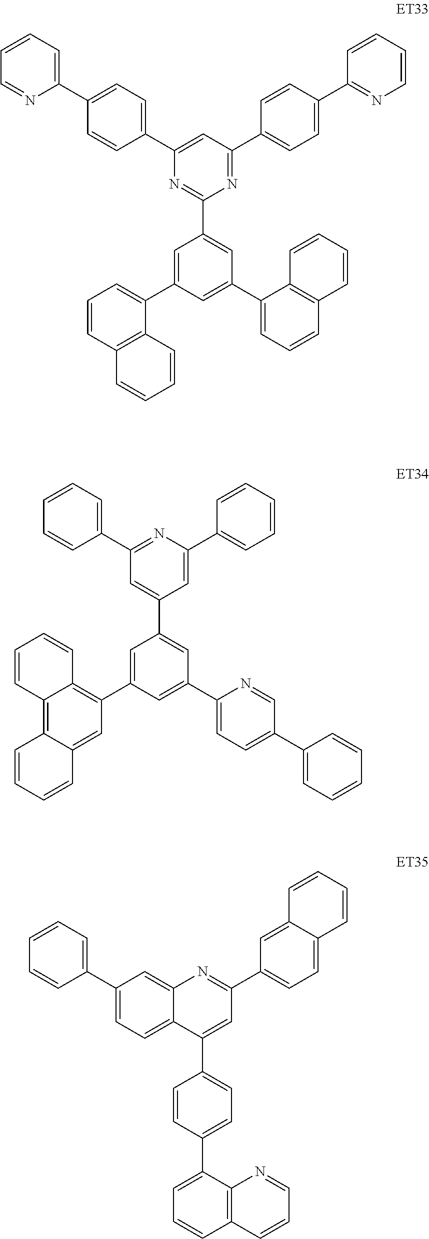 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1]

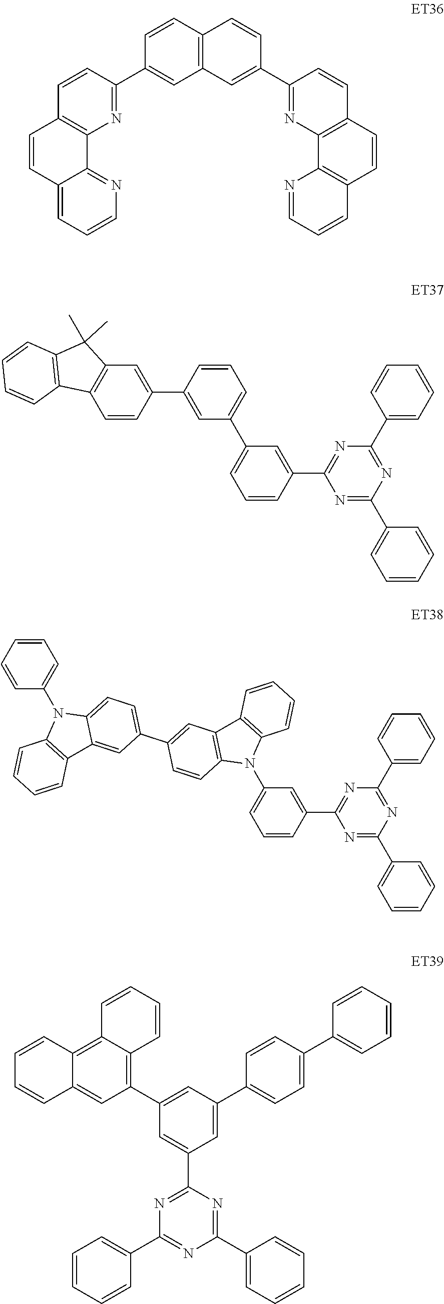 [SMILES: c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

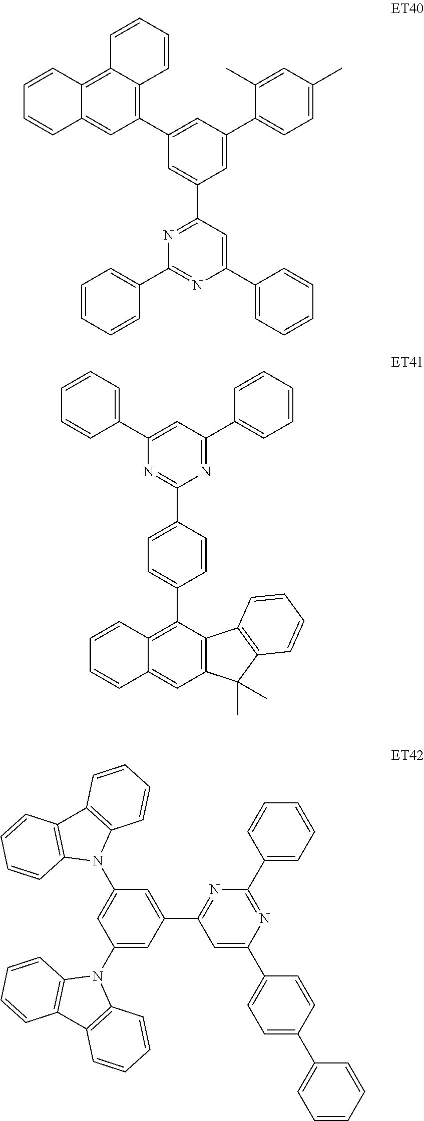

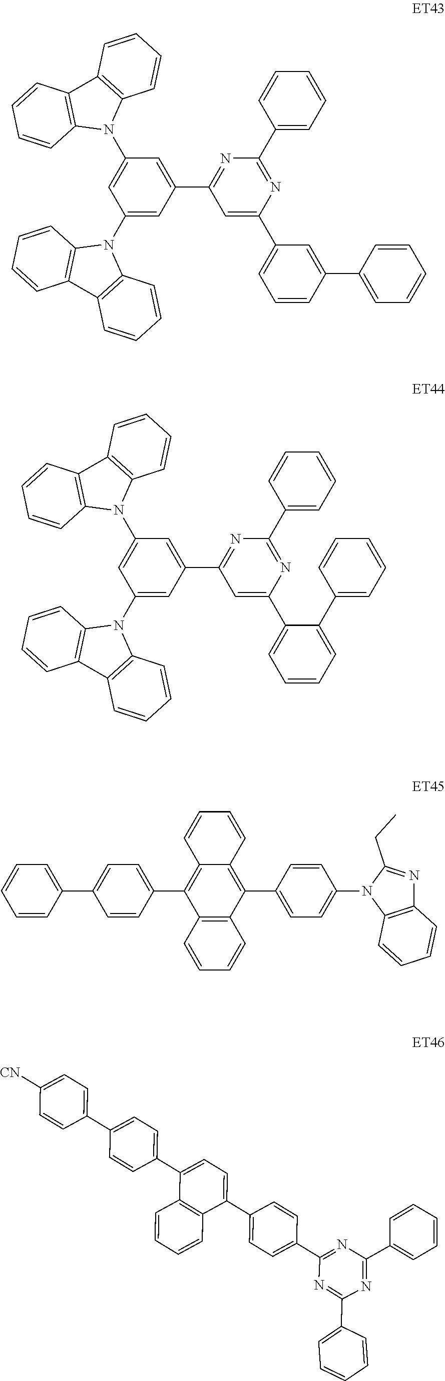

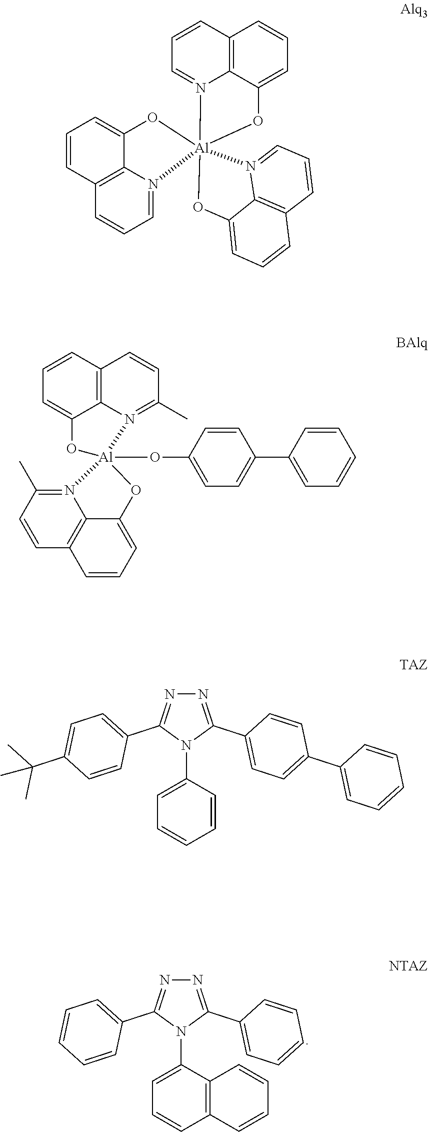

A thickness of the electron transport region may be about 100 Å to about 5,000 Å, for example, about 160 Å to about 4,000 Å. When the electron transport region includes the hole-blocking layer, the electron transport layer, or a combination thereof, a thickness of the hole-blocking layer or electron transport layer may each independently be about 20 Å to about 1,000 Å, for example, about 30 Å to about 300 Å, and the thickness of the electron transport layer may be about 100 Å to about 1,000 Å, for example, about 150 Å to about 500 Å. When the thicknesses of the hole-blocking layer and/or the electron transport layer are within these ranges, satisfactory electron transporting characteristics may be obtained without a substantial increase in driving voltage.

The electron transport region (e.g., an electron transport layer in the electron transport region) may further include, in addition to the aforementioned materials, a metal-containing material.

The metal-containing material may include an alkali metal complex, an alkaline earth metal complex, or a combination thereof. A metal ion of the alkali metal complex may be a Li ion, a Na ion, a K ion, a Rb ion, a Cs ion, or a combination thereof; and a metal ion of the alkaline earth metal complex may be a Be ion, a Mg ion, a Ca ion, a Sr ion, a Ba ion, or a combination thereof. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or a combination thereof.

In one or more embodiments, the metal-containing material may include a Li complex. The Li complex may include, for example, Compound ET-D1 (UQ) or ET-D2, but embodiments are not limited thereto:

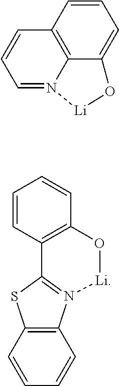

The electron transport region may include an electron injection layer that facilitates the injection of electrons from the second electrode 150. The electron injection layer may directly contact the second electrode 150.

The electron injection layer may have: i) a single-layer structure consisting of a single layer consisting of a single material, ii) a single-layer structure consisting of a single layer consisting of multiple layers that are different from each other, or iii) a multi-layer structure including multiple layers including multiple materials that are different from each other.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a combination thereof.

The alkali metal may include Li, Na, K, Rb, Cs, or a combination thereof. The alkaline earth metal may include Mg, Ca, Sr, Ba, or a combination thereof. The rare earth metal may include Sc, Y, Ce, Tb, Yb, Gd, or a combination thereof.

The alkali metal-containing compound, the alkaline earth metal-containing compound, and the rare earth metal-containing compound may include oxides, halides (for example, fluorides, chlorides, bromides, iodides, or the like), or tellurides of the alkali metal, the alkaline earth metal, and the rare earth metal, or a combination thereof.

The alkali metal-containing compound may include alkali metal oxides, such as Li₂O, Cs₂O, K₂O, or the like, or a combination thereof; alkali metal halides, such as LiF, NaF, CsF, KF, Lil, Nal, Csl, KI, or the like, or a combination thereof; or a combination thereof. The alkaline earth metal-containing compound may include an alkaline earth metal compound, such as BaO, SrO, CaO, BaxSr₁₋ₓO (x is a real number satisfying 0<x<1), BaₓCa₁₋ₓO (x is a real number satisfying 0<x<1), or the like, or a combination thereof. The rare earth metal-containing compound may include YbF₃, ScF₃, Sc₂O₃, Y₂O₃, Ce₂O₃, GdF₃, TbF₃, YbI₃, SCI₃, TbI₃, or the like, or a combination thereof. In one or more embodiments, the rare earth metal-containing compound may include a lanthanide metal telluride. Non-limiting examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, or the like, or a combination thereof.

The alkali metal complex, the alkaline earth-metal complex, and the rare earth metal complex may include i) one of ions of the alkali metal, the alkaline earth metal, and the rare earth metal, and ii) a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or a combination thereof.

In one or more embodiments, the electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a combination thereof, as described above. In another embodiment, the electron injection layer may further include an organic material (e.g., the compound represented by Formula 601).

In another embodiment, the electron injection layer may consist of i) an alkali metal-containing compound (e.g., an alkali metal halide), or ii) a) an alkali metal-containing compound (e.g., an alkali metal halide), and b) an alkali metal, an alkaline earth metal, a rare earth metal, or a combination thereof. In one or more embodiments, the electron injection layer may be a KI:Yb co-deposited layer, an Rbl:Yb co-deposited layer, a UF:Yb co-deposited layer, or the like, but embodiments are not limited thereto.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or a combination thereof may be uniformly or non-uniformly dispersed in a matrix including the organic material.

A thickness of the electron injection layer may be about 1 Å to about 100 Å, and, for example, about 3 Å to about 90 Å. When the thickness of the electron injection layer is within this range, satisfactory electron injection characteristics may be obtained without a substantial increase in driving voltage.

### Second Electrode 150

The second electrode 150 is arranged on the interlayer 130. The second electrode 150 may be a cathode, which is an electron injection electrode, and as a material for forming the second electrode 150, a metal, an alloy, an electrically conductive compound, or a combination thereof, each having a low-work function, may be used.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or a combination thereof. The second electrode 150 may be a transmissive electrode, a transflective electrode, or a reflective electrode.

The second electrode 150 may have a single-layer structure or a multilayer structure including a plurality of layers.

### Capping Layer

A first capping layer (not shown in FIG. 1) may be arranged outside the first electrode 110, and/or a second capping layer (not shown in FIG. 1) may be arranged outside the second electrode 150. In particular, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in the emission layer of the interlayer 130 of the light-emitting device 10 may be extracted toward the outside through the first electrode 110 which is a transflective electrode or a transmissive electrode, and the first capping layer. Light generated in the emission layer of the interlayer 130 of the light-emitting device 10 may be extracted toward the outside through the second electrode 150 which is a transflective electrode or a transmissive electrode, and the second capping layer.

The first capping layer and the second capping layer may increase external emission efficiency according to the principle of constructive interference. Accordingly, the light extraction efficiency of the light-emitting device 10 is increased, such that the luminescence efficiency of the light-emitting device 10 may be increased.

Each of the first capping layer and the second capping layer may include a material having a refractive index of about 1.6 or more (at 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer or the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or a combination thereof. The carbocyclic compound, the heterocyclic compound, and the amine group-containing compound may optionally be substituted with a substituent including O, N, S, Se, Si, F, Cl, Br, I, or a combination thereof.

In one or more embodiments, at least one of the first capping layer and the second capping layer may each independently include an amine group-containing compound.

In another embodiment, at least one of the first capping layer or the second capping layer may each independently include a compound represented by Formula 201, a compound represented by Formula 202, or a combination thereof.

In another embodiment, at least one of the first capping layer or the second capping layer may each independently include one of Compounds HT28 to HT33, one of Compounds CP1 to CP6, β-NPB, or a combination thereof:

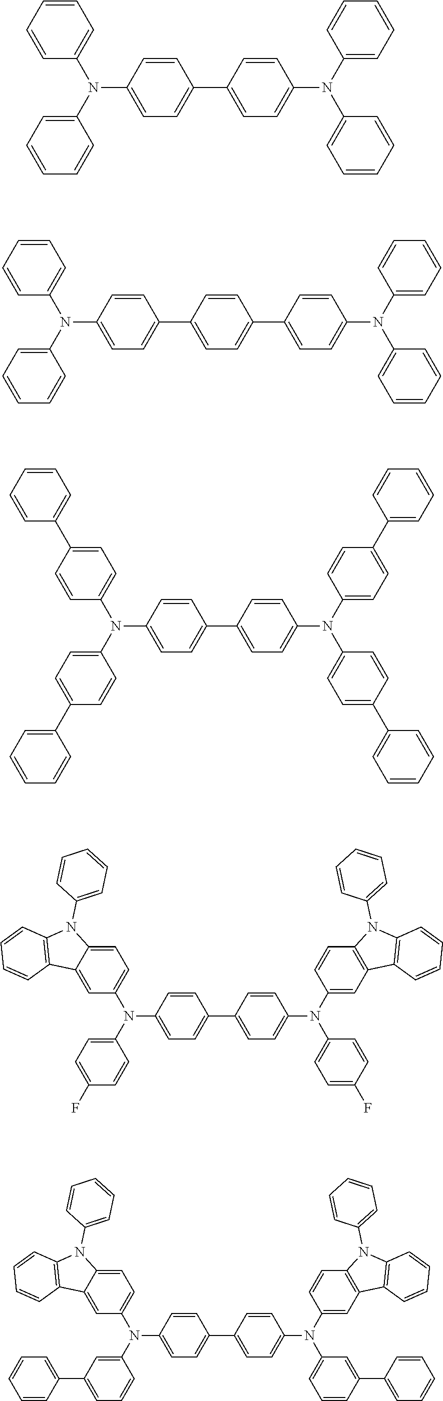 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

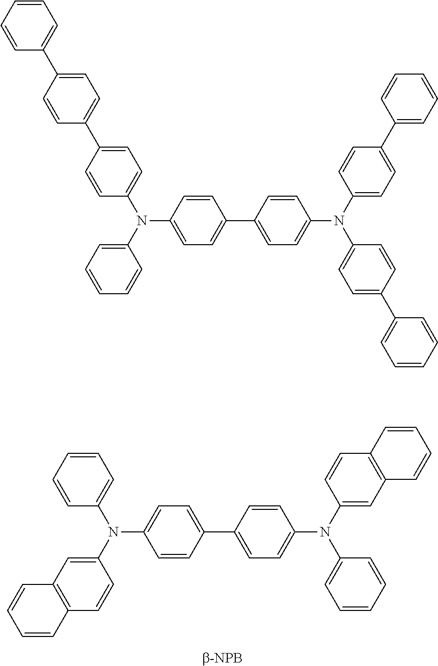 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Electronic Apparatus

The light-emitting device may be included in various electronic apparatuses. For example, the electronic apparatus including the light-emitting device may be a light-emitting apparatus, an authentication apparatus, or the like.

The electronic apparatus (e.g., a light-emitting apparatus) may further include i) a color filter, ii) a color conversion layer, or iii) both a color filter and a color conversion layer, in addition to the light-emitting device. The color filter and/or the color conversion layer may be arranged in at least one traveling direction of light emitted from the light-emitting device. For example, the light emitted from the light-emitting device may be a blue light. A description of the light-emitting device may be referred to the description above. In one or more embodiments, the color conversion layer may include quantum dots.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The plurality of color filter areas (or the plurality of color conversion areas) may include a first area emitting a first color light, a second area emitting a second color light, and/or a third area emitting a third color light, wherein the first color light, the second color light, and/or the third color light may have different maximum emission wavelengths. In one or more embodiments, the first color light may be a red light, the second color light may be a green light, and the third color light may be a blue light. In one or more embodiments, the plurality of color filter areas (or the plurality of color conversion areas) may include quantum dots. In detail, the first area may include red quantum dots, the second area may include green quantum dots, and the third area may not include quantum dots. The first area, the second area, and/or the third area may each further include a scatterer.

In one or more embodiments, the light-emitting device may emit a first light, the first area may absorb the first light to emit a first-1 color light, the second area may absorb the first light to emit a second-1 color light, and the third area may absorb the first light to emit a third-1 color light. In this case, the first-1 color light, the second-1 color light, and the third-1 color light may have different maximum emission wavelengths. In detail, the first light may be a blue light, the first-1 color light may be a red light, the second-1 color light may be a green light, and the third-1 color light may be a blue light.

The electronic apparatus may further include a thin-film transistor, in addition to the light-emitting device as described herein. The thin-film transistor may include a source electrode, a drain electrode, and an active layer, wherein any one of the source electrode or the drain electrode may be electrically connected to any one of the first electrode or the second electrode of the light-emitting device.

The thin-film transistor may further include a gate electrode, a gate insulating film, or the like.

The active layer may include crystalline silicon, amorphous silicon, an organic semiconductor, an oxide semiconductor, or the like.

The electronic apparatus may further include a sealing portion for sealing the light-emitting device. The sealing portion may be arranged between the color filter and/or the color conversion layer and the light-emitting device. The sealing portion allows light from the light-emitting device to be extracted to the outside, and simultaneously prevents ambient air and/or moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

Various functional layers may be additionally arranged on the sealing portion, in addition to the color filter and/or the color conversion layer, according to the use of the electronic apparatus. Examples of the functional layers may include a touch screen layer and a polarizing layer. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, or the like).

The authentication apparatus may further include, in addition to the light-emitting device as described above, a biometric information collector.

The electronic apparatus may be applied to various displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, endoscope displays, or the like), fish finders, various measuring instruments, meters (for example, meters for a vehicle, an aircraft, and a vessel), projectors, or the like.

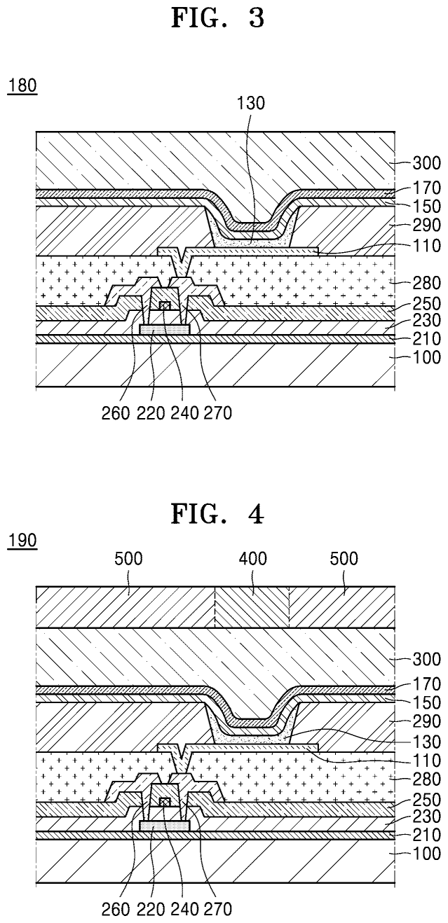

### Description of FIGS. 3 and 4

FIG. 3 is a cross-sectional view of an electronic apparatus 180 according to one or more embodiments.

The electronic apparatus of FIG. 3 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be arranged on the substrate 100. The buffer layer 210 may prevent penetration of impurities through the substrate 100 and may provide a flat surface on the substrate 100.

A TFT may be arranged on the buffer layer 210. The TFT may include an active layer 220, a gate electrode 240, a source electrode 260, and a drain electrode 270.

The active layer 220 may include an inorganic semiconductor, such as silicon or polysilicon, an organic semiconductor, an oxide semiconductor, or the like, and may include a source region, a drain region, and a channel region.

A gate insulating film 230 for insulating the active layer 220 from the gate electrode 240 may be arranged on the active layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 and between the gate electrode 240 and the drain electrode 270, to insulate these electrodes from one another.

The source electrode 260 and the drain electrode 270 may be arranged on the interlayer insulating film 250. The interlayer insulating film 250 and the gate insulating film 230 may be formed to expose the source region and the drain region of the active layer 220, and the source electrode 260 and the drain electrode 270 may be arranged in contact with the exposed portions of the source region and the drain region of the active layer 220.

The TFT may be electrically connected to a light-emitting device to drive the light-emitting device, and may be covered and protected by a passivation layer 280. The passivation layer 280 may include an inorganic insulating film, an organic insulating film, or a combination thereof. A light-emitting device may be provided on the passivation layer 280. The light-emitting device may include the first electrode 110, the interlayer 130, and the second electrode 150.

The first electrode 110 may be arranged on the passivation layer 280. The passivation layer 280 may be arranged to expose a portion of the drain electrode 270, not fully covering the drain electrode 270, and the first electrode 110 may be arranged to be connected to the exposed portion of the drain electrode 270.

A pixel-defining film 290 including an insulating material may be arranged on the first electrode 110. The pixel-defining film 290 may expose a certain region of the first electrode 110, and the interlayer 130 may be formed in the exposed region of the first electrode 110. The pixel-defining film 290 may be a polyimide-containing organic film or a polyacrylic organic film. Although not shown in FIG. 4, at least some layers of the interlayer 130 may extend beyond the upper portion of the pixel defining layer 290 and may thus be located in the form of a common layer.

The second electrode 150 may be arranged on the interlayer 130, and a capping layer 170 may be additionally formed on the second electrode 150. The capping layer 170 may be formed to cover the second electrode 150.

The encapsulation portion 300 may be located on the capping layer 170. The encapsulation portion 300 may be disposed on a light-emitting device to protect the light-emitting device from moisture and/or oxygen. The encapsulation portion 300 may include an inorganic film including silicon nitride (SiNₓ), silicon oxide (SiOₓ), indium tin oxide, indium zinc oxide, or the like, or a combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, or the like), an epoxy-containing resin (for example, aliphatic glycidyl ether (AGE), or the like), or a combination thereof; or a combination of the inorganic film and the organic film.

FIG. 4 is a cross-sectional view of an electronic apparatus 190 according to another embodiment.

The electronic apparatus of FIG. 4 is the same as the electronic apparatus of FIG. 3, except that a light-shielding pattern 500 and a functional region 400 are additionally arranged on the encapsulation portion 300. The functional region 400 may be i) a color filter area, ii) a color conversion area, or iii) a combination of the color filter area and the color conversion area. In one or more embodiments, the light-emitting device included in the light-emitting apparatus of FIG. 4 may be a tandem light-emitting device.

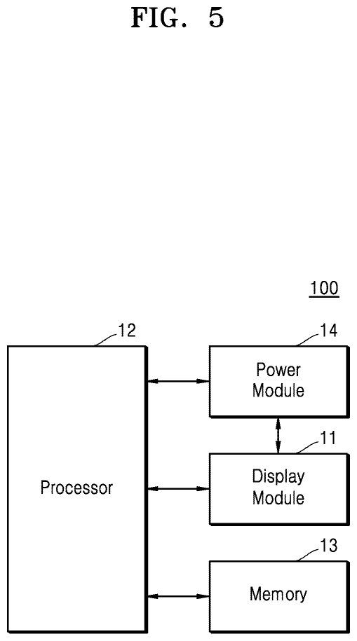

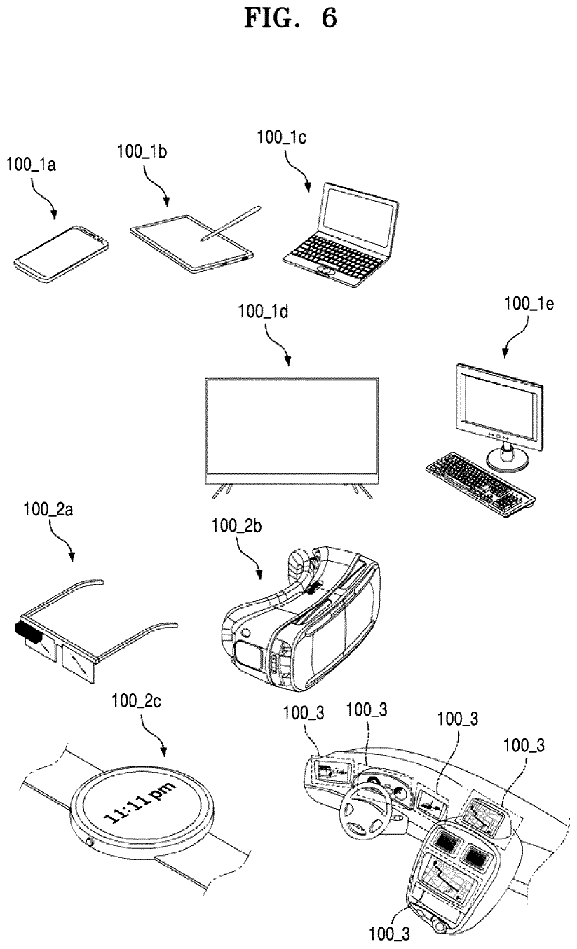

### Descriptions of FIGS. 5 and 6

The light-emitting device according to one or more embodiments may be applied to various electronic apparatuses. The electronic apparatus according to one or more embodiments includes the aforementioned light-emitting device, and may further include a module or apparatus having additional functions, in addition to the light-emitting device.

FIG. 5 is a block view of an electronic apparatus 100 according to one or more embodiments. Referring to FIG. 5, the electronic apparatus 100 according to one or more embodiments may include a display module 11, a processor 12, a memory 13, and a power module 14.

The processor 12 may include at least one of a central processing unit (CPU), an application processor (AP), a graphic processing unit (GPU), a communication processor (CP), an image signal processor (ISP), or a controller.

Data information necessary for the operation of the processor 12 or the display module 11 may be stored in the memory 13. When the processor 12 executes an application stored in the memory 13, an image data signal and/or an input control signal is transmitted to the display module 11, and the display module 11 may process the received signal and output image information through a display screen.

The power module 14 may include: a power supply module, such as a power adapter or a battery device; and a power conversion module that converts power supplied by the power supply module to generate power required for the operation of the electronic apparatus 100.

At least one of components of the electronic apparatus 100 may be included in the light-emitting device according to the aforementioned embodiments. In addition, some of individual modules functionally included in a single module may be included in the light-emitting device, and others may be provided separately from the light-emitting device. For example, the light-emitting device may include the display module 11, and the processor 12, the memory 13, and the power module 14 may be provided in the form of an apparatus other than the light-emitting device within the electronic apparatus 100.

FIG. 6 is a schematic view of an electronic apparatus according to various embodiments.

Referring to FIG. 6, various electronic apparatuses to which the light-emitting device according to embodiments is applied may include not only an electronic apparatus for displaying images, such as a smartphone 100_1a, a tablet PC 100_1b, a laptop 100_1c, a TV 100_1d, a desk monitor 100_1e, or the like, but also a wearable electronic apparatus including a light-emitting module, such as smart glasses 100_2a, a head-mounted display 100_2b, a smart watch 100_2c, or the like, and an electronic apparatus including a display module 100_3 including an automobile dashboard, a center fascia, a center information display (CID) on a dashboard, a room mirror display, or the like, but embodiments are not limited thereto.

### Manufacturing Method

Layers constituting the hole transport region, the emission layer, and the layers constituting the electron transport region may be formed in a certain region by using various methods such as vacuum deposition, spin coating, casting, Langmuir-Blodgett (LB) deposition, ink-jet printing, laser-printing, laser-induced thermal imaging, or the like.

When the layers constituting the hole transport region, the emission layer, and the layers constituting the electron transport region are formed by vacuum deposition, the deposition may be performed at a deposition temperature of about 100° C. to about 500° C., at a vacuum degree of about 10⁻⁸ torr to about 10⁻³ torr, and at a deposition speed of about 0.01 angstroms per second (Å/sec) to about 100 Å/sec, depending on a material to be included in a layer to be formed and the structure of a layer to be formed.

When layers constituting the hole transport region, an emission layer, and layers constituting the electron transport region are formed by spin coating, the spin coating may be performed at a coating speed of about 2,000 revolutions per minute (rpm) to about 5,000 rpm, and at a heat treatment temperature of about 80° C. to about 200° C. by taking into account a material to be included in a layer to be formed and the structure of a layer to be formed.

### Definition of Terms

The term “C₃-C₆₀ carbocyclic group” as used herein refers to a cyclic (ring) group consisting of carbon atoms as the only ring-forming atoms and having three to sixty carbon atoms, and the term “C₁-C₆₀ heterocyclic group” as used herein refers to a cyclic (ring) group that has one to sixty carbon atoms and further includes, in addition to a carbon atom as ring-forming atom(s), at least one heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. In one or more embodiments, the number of ring-forming atoms of the C₁-C₆₀ heterocyclic group may be 3 to 61.

The “cyclic group” as used herein may include both the C₃-C₆₀ carbocyclic group and the C₁-C₆₀ heterocyclic group.

The term “π electron-rich C₃-C₆₀ cyclic group” as used herein refers to a cyclic group that has three to sixty carbon atoms as ring-forming atoms and does not include *—N═*′ as a ring-forming moiety, and the term “π electron-deficient nitrogen-containing C₁-C₆₀ cyclic group” as used herein refers to a heterocyclic group that has one to sixty carbon atoms as ring-forming atoms, and includes *—N═*′ as a ring-forming moiety.

In one or more embodiments,
the C₃-C₆₀ carbocyclic group may be i) Group T1, or ii) a condensed cyclic group in which two or more of Group T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, an indenoanthracene group, or the like),
the C₁-C₆₀ heterocyclic group may be i) Group T2, ii) a condensed cyclic group in which two or more of Group T2 are condensed with each other, or iii) a condensed cyclic group in which at least one of Group T2 and at least one of Group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, or the like),
the π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one of Group T3 and at least one of Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, or the like),
the π electron-deficient nitrogen-containing C₁-C₆₀ cyclic group may be i) Group T4, ii) a condensed cyclic group in which two or more of Group T4 are condensed with each other, iii) a condensed cyclic group in which at least one of Group T4 and at least one of Group T1 are condensed with each other, iv) a condensed cyclic group in which at least one of Group T4 and at least one of Group T3 are condensed with each other, or v) a condensed cyclic group in which at least one of Group T4, at least one of Group T1, and at least one of Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, or the like),
Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,
Group T2 may be a furan group, a thiophene group, a 1H-pyrrole group, a silole group, a borole group, a 2H-pyrrole group, a 3H-pyrrole group, an imidazole group, a pyrazole group, a trazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyidine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
Group T3 may be a furan group, a thiophene group, a 1H-pyrrole group, a silole group, or a borole group, and
Group T4 may be a 2H-pyrrole group, a 3H-pyrrole group, an imidazole group, a pyrazole group, a trazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group”, “C₃-C₆₀ carbocyclic group”, “C₁-C₆₀ heterocyclic group”, “Tr electron-rich C₃-C₆₀ cyclic group”, or “Tr electron-deficient nitrogen-containing C₁-C₆₀ cyclic group” as used herein may refer to a group condensed to any cyclic group, a monovalent group, or a polyvalent group (for example, a divalent group, a trivalent group, a tetravalent group, or the like) according to the structure of a formula for which the corresponding term is used. In one or more embodiments, “a benzene group” may be a benzo group, a phenyl group, a phenylene group, or the like, which may be easily understand by one of ordinary skill in the art according to the structure of a formula including the “benzene group.”

Non-limiting examples of a monovalent C₃-C₆₀ carbocyclic group and a monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and non-limiting examples of a divalent C₃-C₆₀ carbocyclic group and a divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has one to sixty carbon atoms, and non-limiting examples thereof include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, or the like. The term “C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and non-limiting examples thereof include an ethenyl group, a propenyl group, a butenyl group, or the like. The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and non-limiting examples thereof include an ethynyl group, a propynyl group, or the like. The term “02-C₆₀ alkynylene group” as used herein refers to a divalent group having the same structure as the 02-Co alkynyl group.

The term “C₁-C₆₀ alkoxy group” as used herein refers to a monovalent group represented by —OA₁₀₁ (wherein A₁₀₁ is the C₁-C₆₀ alkyl group), and non-limiting examples thereof include a methoxy group, an ethoxy group, an isopropyloxy group, or the like.

The term “C₁-C₆₀ alkylthio group” as used herein refers to a monovalent group represented by —SA_{101′} (wherein A_{101′} is the C₁-C₆₀ alkyl group).

The term “C₃-C₁₀ cycloalkyl group” as used herein refers to a monovalent saturated hydrocarbon group including 3 to 10 carbon atoms. Non-limiting examples of the C₃-C₁₀ cycloalkyl group include a cyclopropyl group, a cyclobutyl group, a cyclopentyl group, a cyclohexyl group, a cycloheptyl group, a cyclooctyl group, an adamantyl group, a norbornyl (bicyclo[2.2.1]heptyl) group, a bicyclo[1.1.1]pentyl group, a bicyclo[2.1.1]hexyl group, a bicyclo[2.2.2]octyl group, or the like. The term “C₃-C₁a cycloalkylene group” as used herein refers to a divalent group having the same structure as the C₃-C₁₀ cycloalkyl group.

The term “C₁-C₁₀ heterocycloalkyl group” as used herein refers to a monovalent cyclic group of 1 to 10 carbon atoms, further including, in addition to carbon atoms as ring-forming atom(s), at least one heteroatom as ring-forming atoms, and non-limiting examples thereof may include a 1,2,3,4-oxatriazolidinyl group, a tetrahydrofuranyl group, a tetrahydrothienyl group, or the like. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent cyclic group that has three to ten carbon atoms as ring-forming atoms and at least one carbon-carbon double bond in the ring thereof and no aromaticity, and non-limiting examples thereof include a cyclopentenyl group, a cyclohexenyl group, a cycloheptenyl group, or the like. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group that has one to ten carbon atoms as ring-forming atom(s), further includes, in addition to the carbon atoms, at least one heteroatom as a ring-forming atom, and has at least one double bond in the ring thereof. Non-limiting examples of the C₁-C₁₀ heterocycloalkenyl group may include a 4,5-dihydro-1,2,3,4-oxatriazolyl group, a 2,3-dihydrofuranyl group, a 2,3-dihydrothienyl group, or the like. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic ring system of six to sixty carbon atoms, and the term “C₆-C₆₀ arylene group” as used herein refers to a divalent group having a carbocyclic aromatic ring system of six to sixty carbon atoms. Non-limiting examples of the C₆-C₆₀ aryl group include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, or the like. When the C₆-C₆₀ aryl group and the C₆-C₆₀ arylene group each include two or more rings, the two or more rings may be condensed with each other.

The term “C₁-C₆₀ heteroaryl group” as used herein refers to a monovalent group having a heterocyclic aromatic ring system that has one to sixty carbon atoms as ring-forming atom(s) and further includes, in addition to the carbon atom(s), at least one heteroatom as a ring-forming atom. The term “C₁-C₆₀ heteroarylene group” as used herein refers to a divalent group having a heterocyclic aromatic ring system that has one to sixty carbon atoms and further includes, in addition to the carbon atom(s), at least one heteroatom as a ring-forming atom. Non-limiting examples of the C₁-C₆₀ heteroaryl group include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, or the like. When the C₁-C₆₀ heteroaryl group and the C₁-C₆₀ heteroarylene group each include two or more rings, the two or more rings may be condensed with each other.

The term “monovalent non-aromatic condensed polycyclic group” as used herein refers to a monovalent group having two or more rings condensed with each other, only carbon atoms (for example, eight to sixty carbon atoms) as ring-forming atoms, and no aromaticity in its molecular structure when considered as a whole. Non-limiting examples of the monovalent non-aromatic condensed polycyclic group include an indenyl group, a fluorenyl group, a spiro-bifluorenyl group, a benzofluorenyl group, an indenophenanthrenyl group, an indeno anthracenyl group, or the like. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group that has two or more rings condensed with each other, further includes, in addition to carbon atoms (for example, one to sixty carbon atoms), at least one heteroatom as a ring-forming atom, and has no aromaticity in its molecular structure when considered as a whole. Non-limiting examples of the monovalent non-aromatic condensed heteropolycyclic group include an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, or the like. The term “divalent non-aromatic condensed heteropolycyclic group” as used herein refers to a divalent group having the same structure as the monovalent non-aromatic condensed heteropolycyclic group.

The term “C₆-C₆₀ aryloxy group” as used herein refers to —OA₁₀₂ (wherein A₁₀₂ is the C₆-C₆₀ aryl group), and the term “C₆-C₆₀ arylthio group” as used herein refers to —SA₁₀₃ (wherein A₁₀₃ is the C₆-C₆₀ aryl group).

The term “C₁-C₆₀ heteroaryloxy group” as used herein refers to —OA_{102′} (wherein A_{102′} is the C₁-C₆₀ heteroaryl group), and the term “C₁-C₆₀ heteroarylthio group” as used herein refers to —SA_{103′} (wherein A_{103′} is the C₁-C₆₀ heteroaryl group).

The term “C₇-C₆₀ aryl alkyl group” as used herein refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroaryl alkyl group” as used herein refers to -A₁₀₆A₁₀₇ (wherein A₁₀₆ is a C₁-C₅₉ alkylene group, and A₁₀₇ is a C₁-C₅₉ heteroaryl group).

Unless otherwise defined, the term “R₁₀ₐ” as used herein refers to:
deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, or a phosphoric acid group or a salt thereof;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)(Q₁₁), —S(═O)₂(Q₁₁), —P(Q₁₁)(Q₁₂), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ aryl alkyl group, or a C₂-C₆₀ heteroaryl alkyl group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)(Q₂₁), —S(═O)₂(Q₂₁), —P(Q₂₁)(Q₂₂), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)(Q₃₁), —S(═O)₂(Q₃₁), —P(Q₃₁)(Q₃₂), or —P(═O)(Q₃₁)(Q₃₂).

Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, or
a C₃-C₆₀ carbocyclic group, a C₁-C₆₀ heterocyclic group, a C₇-C₆₀ aryl alkyl group, a C₂-C₆₀ heteroarylalkyl group, a C₆-C₆₀ aryloxy group, a C₆-C₆₀ arylthio group, a C₁-C₆₀ heteroaryloxy group, or a C₁-C₆₀ heteroarylthio group,
each unsubstituted or substituted with deuterium, —F, cyano group, a C₁-C₆₀ alkyl group, a C₁-C₆₀ alkoxy group, a C₁-C₆₀ alkylthio group, a phenyl group, a biphenyl group, or a combination thereof.

The term “heteroatom” as used herein refers to any atom other than a carbon atom. Examples of the heteroatom include O, S, N, P, Si, B, Ge, Se, or a combination thereof.

The term “transition metal” as used herein may include titanium (Ti), cobalt (Co), copper (Cu), zinc (Zn), zirconium (Zr), ruthenium (Ru), rhodium (Rh), palladium (Pd), rhenium (Re), hafnium(Hf), tantalum (Ta), tungsten (W), osmium (Os), iridium (Ir), platinum (Pt), or gold (Au).

The term “Ph” as used herein refers to a phenyl group, the term “Me” as used herein refers to a methyl group, the term “Et” as used herein refers to an ethyl group, the term “ter-Bu” or “Bu^{t}” as used herein refers to a tert-butyl group, and the term “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

The number of carbon atoms in the substituent definition is exemplary. For example, in the C₁-C₆₀ alkyl group, the number of carbon atoms, 60, is an exemplary, and the definition for the alkyl group is equally applied to the C₁-C₂₀ alkyl group. The other cases are the same.

Any hydrogen in the compound structures described herein may optionally be substituted with deuterium.

* and *′ as used herein, unless defined otherwise, each refer to a binding site to a neighboring atom in a corresponding formula.

Hereinafter, a light-emitting device according to embodiments will be described in further detail with reference to Examples.

### EXAMPLES

### Synthesis Example 1 (Compound 37)

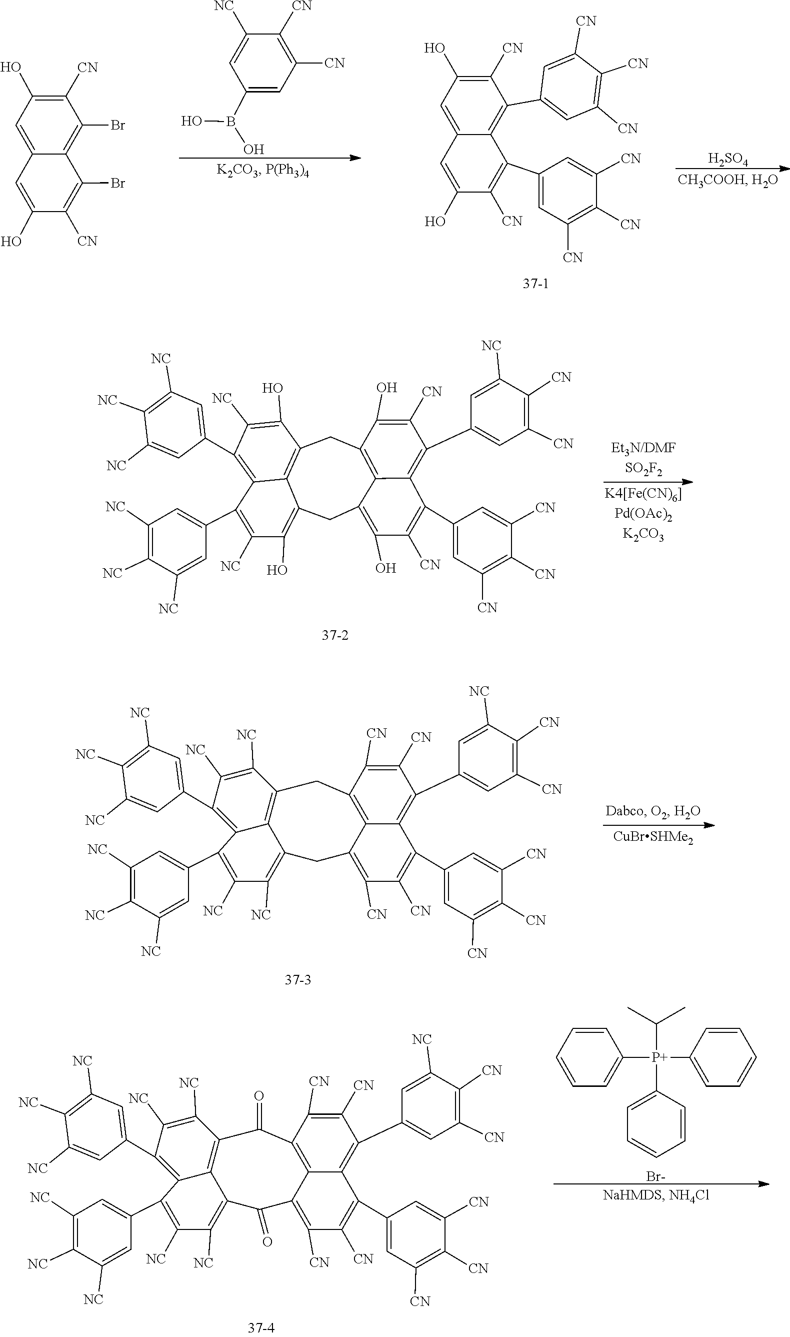

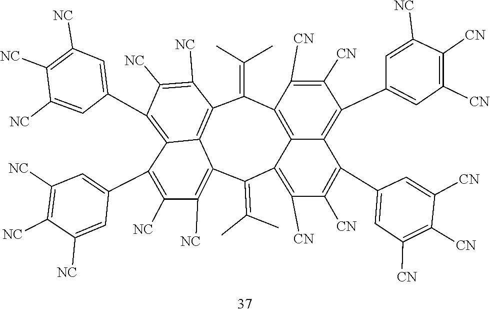 [SMILES: CC(C)=c1c2c(C#N)c(C#N)c(-c3cc(C#N)c(C#N)c(C#N)c3)c3c(-c4cc(C#N)c(C#N)c(C#N)c4)c(C#N)c(C#N)c(c(=C(C)C)c4c(C#N)c(C#N)c(-c5cc(C#N)c(C#N)c(C#N)c5)c5c(-c6cc(C#N)c(C#N)c(C#N)c6)c(C#N)c(C#N)c1c54)c32]

Synthesis Example 1-1) Synthesis of Intermediate 37-1: 1,8-dibromo-3,6-dihydroxynaphthalene-2,7-dicarbonitrile (1 equivalent (eq.)), (3,4,5-tricyanophenyl)boronic acid (2.2 eq.), potassium carbonate (20 eq.), and tetrakis(triphenylphosphine)palladium(0) (Pd(PPh₃)₄) (0.2 eq.) were added to 150 milliliters (mL) of toluene, 40 mL of ethanol, and 15 mL of H₂O, and the mixed solution was stirred at 90° C. for 12 hours. After completion of the reaction, followed by an extraction process, the resulting product was purified by column chromatography to obtain 4.12 g of Intermediate 37-1. (Yield: 59.2%)

Synthesis Example 1-2) Synthesis of Intermediate 37-2: 37-1 (1 eq.) and paraformaldehyde (1.5 eq.) were added to 30 mL of acetic acid and 0.2 mL of sulfuric acid, and the contents were reacted at room temperature for 12 hours. The precipitates were filtered, and the filtrate was washed with acetic acid and H₂O, and then dried to obtain 2.35 g of Intermediate 37-2. (Yield: 40%).

Synthesis Example 1-3) Synthesis of Intermediate 37-3: Intermediate 37-2 (1 eq.) and Et₃N (8 eq.) were dissolved in 20 mL of dimethylformamide (DMF) in a flask, and a balloon filled with SO₂F₂ (20 eq.) gas was attached to the flask. K₄[Fe(CN)₆]·3H₂O (8 eq.), palladium acetate (Pd(OAc)₂) (0.2 eq), 2-(diphenylphosphino)-biphenyl (0.4 eq.), and K₂CO₃ (8 eq.) were additionally added to the flask, and the reaction mixture was heated at 100° C. for 12 hours under an inert gas atmosphere. After completion of the reaction, an extraction process was performed using H₂Oand ethyl acetate (EtOAc), the organic layer was separated, followed by removal of moisture with Na₂SO₄, and then removal of the solvent under reduced pressure. The resulting product was purified by column chromatography using pet-ether/EtOAc to obtain 2.54 g of Intermediate 37-3. (Yield: 61%)

Synthesis Example 1-4) Synthesis of Intermediate 37-4: Intermediate 37-3 (1 eq.) was dissolved in 150 mL of dimethylsulfoxide (DMSO), and CuBr—SMe₂ (0.3 eq.) and 1,4-diazabicyclo[2.2.2]octane (Dabco) (2 eq.) were added thereto, the contents were heated at 100° C. for 32 hours in an O₂ atmosphere. The reaction was completed by adding an ammonium buffer solution having a pH of 9, and an extraction process was performed thereon using EtOAc and brine, the organic layer was separated, and MgSO₄ was added thereto to remove moisture. Then, the solvent was removed under reduced pressure. The resulting product was purified by column chromatography using EtOAc and hexane at a ratio of 4:6 to obtain 1.65 g of Intermediate 37-4. (Yield: 64%).

Synthesis Example 1-5) Synthesis of Compound 37: Benzophenone, isopropyltriphenylphosphonium bromide (1.5 eq.), and sodium bis(trimethylsilyl)amide (NaHMDS) (1.5 eq.) were dissolved in 200 mL of tetrahydrofuran (THF), and the contents were held for 3 hours at 0° C. Then Intermediate 37-4 (1 eq.) dissolved in a small amount of THF was added thereto, and the contents were reacted at room temperature for 12 hours. The reaction was completed by adding an NH₄Cl solution, and an extraction process was performed thereon using EtOAc and H₂O. The filtrate was washed with brine, the organic layer was separated, and MgSO₄ was added thereto to remove moisture. Then, the solvent was removed under reduced pressure. The resulting product was purified by column chromatography to obtain 0.73 g of Compound 37. (Yield: 46.5%) Synthesis Example 2 (Compound 1-4)

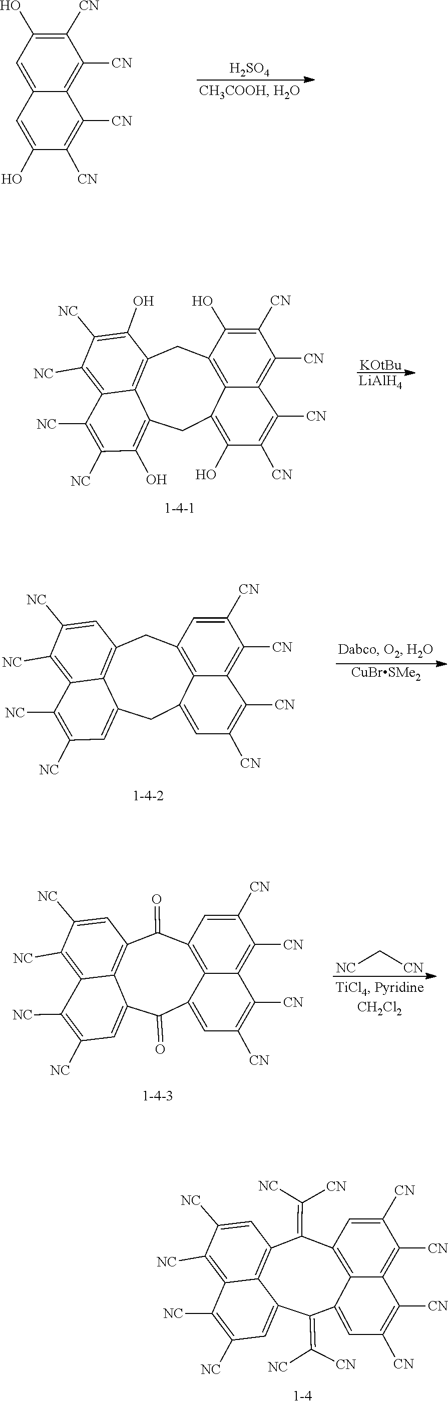

Synthesis Example 2-1) Synthesis of Intermediate 1-4-1: 3,6-dihydroxynaphthalene-1,2,7,8-tetracarbonitrile (1 eq.) and paraformaldehyde (1.5 eq.) were added to 30 mL of acetic acid and 0.2 mL of sulfuric acid, and the contents were reacted at room temperature for 12 hours. The precipitates were filtered, the filtrate was washed with acetic acid and H₂O, and then dried to obtain 5.6 g of Intermediate 1-4-1. (Yield: 54%)

Synthesis Example 2-2) Intermediate 1-4-1 (1 eq.) and KOtBu (10 eq.) were dissolved in 150 mL of anhydrous THF, and a solution containing LiAlH₄ (15 eq.) dissolved in a small amount of anhydrous THF was slowly added thereto, and the contents were reacted at 140° C. for 36 hours. After completion of the reaction, an extraction process was performed thereon using toluene, and the solvent was removed under reduced pressure/heating. The resulting product was dissolved in pet-ether and purified by flash column chromatography to obtain 3.72 g of Intermediate 1-4-2. (Yield: 84.3%)

Synthesis Example 2-3) Synthesis of Intermediate 1-4-3: Intermediate 1-4-2 (1 eq.) was dissolved in 150 mL of DMSO, and CuBr—SMe₂ (0.3 eq.) and Dabco (2 eq.) were added thereto, and the contents were reacted at 100° C. for 32 hours in an 02 atmosphere. The reaction was completed by adding an ammonium buffer solution having a pH of 9, and an extraction process was performed thereon using EtOAc and brine, the organic layer was separated, and MgSO₄ was added thereto to remove moisture. Then, the solvent was removed under reduced pressure. The resulting product was purified by column chromatography using EtOAc and hexane at a ratio of 3:7 to obtain 2.14 g of Intermediate 1-4-3. (Yield: 58%)

Synthesis Example 2-4) Synthesis of Compound 1-4: Intermediate 1-4-3 (1 eq.) and malononitrile (8 eq.) were added to 250 mL of anhydrous CH₂Cl₂, and the contents were reacted for 1 hour in an inert gas atmosphere. TiCl₄ (8 eq.) and pyridine (16 eq.) were sequentially added thereto, and the contents were additionally reacted for 2 hours. The reaction was completed by adding water, and an extraction process was performed thereon using CH₂Cl₂. The filtrate was washed with brine, the organic layer was separated, and MgSO₄ was added thereto to remove moisture. Then, the solvent was removed under reduced pressure. The resulting product was dissolved in a small amount of CH₂Cl₂ and purified using a silica filter to obtain 1.86 g of Compound 1-4. (Yield: 62.6%)

### Synthesis Example 3 (Compound 1-13)

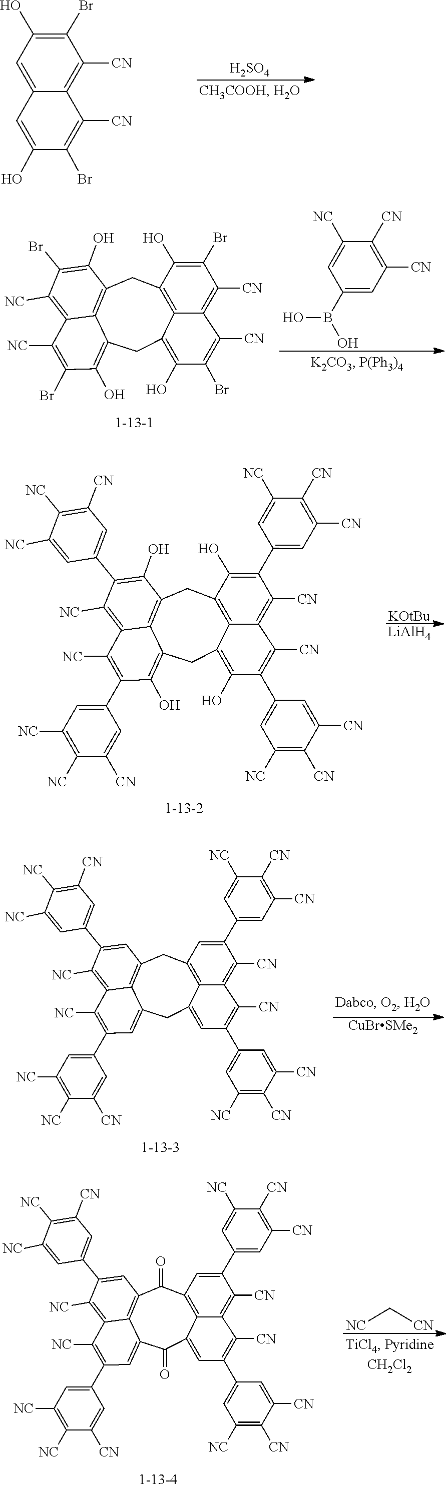

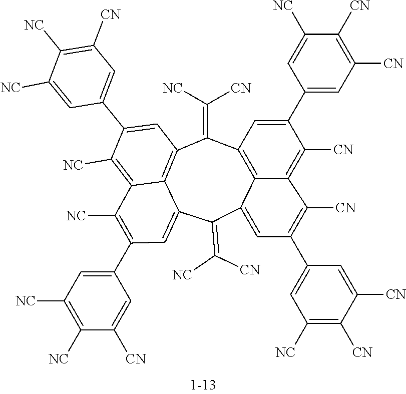 [SMILES: N#CC(C#N)=c1c2cc(-c3cc(C#N)c(C#N)c(C#N)c3)c(C#N)c3c(C#N)c(-c4cc(C#N)c(C#N)c(C#N)c4)cc(c(=C(C#N)C#N)c4cc(-c5cc(C#N)c(C#N)c(C#N)c5)c(C#N)c5c(C#N)c(-c6cc(C#N)c(C#N)c(C#N)c6)cc1c54)c32]

Synthesis Example 3-1) Synthesis of Intermediate 1-13-1: 2,7-dibromo-3,6-dihydroxynaphthalene-1,8-dicarbonitrile (1 eq.) and paraformaldehyde (1.5 eq.) were added to 30 mL of acetic acid and 0.2 mL of sulfuric acid, and the mixed solution was reacted at room temperature for 12 hours. The precipitates were filtered, the filtrate was washed with acetic acid and H₂O, and the product was dried to obtain 7.4 g of Intermediate 1-13-1. (Yield: 72%)

Synthesis Example 3-2) Synthesis of Intermediate 13-1-2: Intermediate 13-1-1 (1 eq.), (3,4,5-tricyanophenyl)boronic acid (2.2 eq.), potassium carbonate (20 eq.), and Pd(PPhs)₄ (0.2 eq.) were added to 150 mL of toluene, 40 mL of ethanol, and 15 mL of H₂O, and the mixed solution was stirred at 90° C. for 12 hours. After completion of the reaction, the resulting product was purified by flash column chromatography to obtain 6.0 g of Intermediate 13-1-2. (Yield: 87%)

Synthesis Example 3-3) Synthesis of Intermediate 13-1-3: Intermediate 13-1-2 (1 eq.) and KOtBu (10 eq.) were dissolved in 200 mL of anhydrous THF, and a solution containing LiAlH₄ (15 eq.) dissolved in a small amount of anhydrous THF was slowly added thereto, and the contents were reacted at 140° C. for 36 hours. After completion of the reaction, an extraction process was performed thereon using toluene, and the solvent of an organic layer obtained by the extraction process was removed under reduced pressure/heating. The resulting product was dissolved in pet-ether and purified by flash column chromatography to obtain 3.22 g of Intermediate 1-13-3. (Yield: 68%)

Synthesis Example 3-4) Synthesis of Intermediate 13-1-4: Intermediate 13-1-3 (1 eq.) was dissolved in 150 mL of DMSO, and CuBr·SMe₂ (0.3 eq.) and Dabco (2 eq.) were added thereto, and the contents were reacted at 100° C. for 32 hours in an 02 atmosphere. The reaction was completed by adding an ammonium buffer solution having a pH of 9, and an extraction process was performed thereon using EtOAc and brine, the organic layer was separated, and MgSO₄ was added thereto to remove moisture. Then, the solvent was removed under reduced pressure. The resulting product was purified by column chromatography using EtOAc and hexane at a ratio of 3:7 to obtain 1.8 g of Intermediate 1-13-4. (Yield: 58%)

Synthesis Example 3-5) Synthesis of Compound 13-1: Intermediate 13-1-4 (1 eq.) and malononitrile (8 eq.) were added to 200 mL of anhydrous CH₂Cl₂, and the contents were reacted for 1 hour in an inert gas atmosphere. TiCl₄ (8 eq.) and pyridine (16 eq.) were sequentially added thereto, and the contents were reacted for an additional 2 hours. The reaction was completed by adding water, and an extraction process was performed thereon using CH₂Cl₂. The filtrate was washed with brine, an organic layer was separated, and MgSO₄ was added thereto to remove moisture. Then, the solvent was removed under reduced pressure. The resulting product was dissolved in a small amount of CH₂Cl₂ and purified using a silica filter to obtain 1.55 g of Compound 1-13. (yield: 78%)

The proton nuclear magnetic resonance spectroscopy (¹H-NMR) and mass spectrometry (MS, fast atom bombardment (FAB)) data for the compounds above are shown in Table 1.

TABLE 1

| | | MS/FAB | |
|---|---|---|---|
| Compound | ¹H NMR (CDCl₃, 500 MHz) | found | calc. |
| 37 | 1.87 (s, 12H), 8.51 (s, 8H) | 1164.11 | 1165.09 |
| 1-4 | 7.56 (s, 4H) | 603.96 | 604.51 |
| 1-13 | 8.45 (s, 8H), 8.07 (m, 4H) | 1108.04 | 1108.98 |

For the compounds above and Compounds 101 and 102, highest occupied molecular orbital (HOMO) energy, lowest unoccupied molecular orbital (LUMO) energy, hole mobility, electron mobility, and glass transition temperature (T_{g}) were measured, and the results thereof are shown in Table 2.

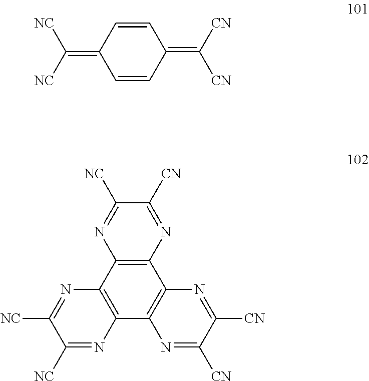 [SMILES: N#CC(C#N)=c1ccc(=C(C#N)C#N)cc1 ; N#Cc1nc2c3nc(C#N)c(C#N)nc3c3nc(C#N)c(C#N)nc3c2nc1C#N]

Evaluation method for HOMO energy level: By using cyclic voltammetry (CV) (electrolyte: 0.1 M Bu₄NPF₆/solvent: DMF/electrode: 3-electrode system (working electrode: GC, reference electrode: Ag/AgCl, and auxiliary electrode: Pt)), the potential (V)-current (A) graph of each compound was obtained and the HOMO energy level of each compound was calculated from the oxidation onset of the graph. The HOMO energy level is reported in electron volts (eV).

Evaluation method for LUMO energy level: By using cyclic voltammetry (CV) (electrolyte: 0.1 M Bu₄NPF₆/solvent: DMF/electrode: 3-electrode system (working electrode: GC, reference electrode: Ag/AgCl, and auxiliary electrode: Pt)), the potential (V)-current (A) graph of each compound was obtained and the LUMO energy level of each compound was calculated from the reduction onset of the graph. The LUMO energy level is reported in eV.

Evaluation methods for hole mobility and electron mobility: Referring to the document “Hole mobility of N,N′-bis(naphtanlen-1-yl)-N,N′-bis(phenyl)benzidine investigated by using space-charge-limited currents,” Appl. Phys. Lett. 90, 203512 (2007), the space-charge-limited current (SCLC) method was used for the evaluation. The hole mobility and the electron mobility are reported as square centimeters per volt second (cm²Ns).

Evaluation method for glass transition temperature: Glass transition temperature (T_{g}, Glass transition temperature) characteristics were analyzed by differential scanning calorimetry (DSC), and the analysis conditions were as follows: a sample was weighed (5 mg) and heated at a scanning rate of 10° C./min from room temperature to 300° C., cooled from 300° C. to 25° C., and heated again to 300° C. Here, the glass transition temperature was measured during the second heating, and obtained from the inflection point on a graph.

TABLE 2

| | | | | | Glass |
|---|---|---|---|---|---|
| | | | Hole | Electron | transition |
| | HOMO | LUMO | mobility | mobility | temperature |
| Compound | (eV) | (eV) | (cm²/Vs) | (cm²/Vs) | (° C.) |
| 37 | −8.51 | −5.10 | 2.87E−07 | 5.29E−05 | 197.8 |
| 1-4 | −8.60 | −5.30 | 4.65E−03 | 3.28E−03 | 140.8 |
| 1-13 | −8.56 | −5.12 | 5.70E−07 | 2.25E−05 | 109.9 |
| 101 | −7.58 | −5.02 | 7.71E−02 | 5.87E−02 | 101.8 |
| 102 | −9.05 | −4.81 | 9.96E−04 | 6.90E−03 | 108.7 |

The compound of Formula 1 according to one or more embodiments is a hole injection compound having a central molecular structure including an octagonal ring containing two naphthalene groups and two ethylene group. As the bulk characteristics of the compound are enhanced, the electron mobility decreases and the glass transition temperature increases, enabling the design of stable materials and reducing lateral leakage in the light-emitting device, thereby improving the device characteristics.

### Manufacture of Light-Emitting Device

### Example 1

As an anode, a glass substrate (Corning Inc.) with a 15 Ω/cm² (1,200 Å) ITO electrode formed thereon was cut to a size of 50 millimeters (mm)×50 mm×0.7 mm, sonicated by using isopropyl alcohol and deionized (DI) water each for 5 minutes, cleaned by irradiation of ultraviolet rays and exposure of ozone thereto for 30 minutes, and then mounted on a vacuum deposition apparatus.

Compound 37 and compound HT3 as p-dopants were vacuum-deposited on the anode at a weight ratio of 3:97 to form a hole injection layer having a thickness of 100 Å, and HT40 was deposited on the hole injection layer to form a hole transport layer having a thickness of 1,250 Å.

Compound H129 as a first host, Compound H130 as a second host, and Compound PD40 as a dopant were co-deposited on the hole transport layer at a weight ratio of 45:45:10 to form an emission layer having a thickness of 300 Å.

Subsequently, Compound ET37 was deposited on the emission layer to form a hole-blocking layer having a thickness of 50 Å, ET46 and LiQ were deposited on the hole-blocking layer at a weight ratio of 5:5 to form an electron transport layer having a thickness of 310 Å, Yb was deposited on the electron transport layer to form an electron injection layer having a thickness of 15 Å, and Ag and Mg were deposited on the electron injection layer at a weight ratio of 5:5 to form a cathode having a thickness of 1,000 Å, thereby completing the manufacture of a light-emitting device.

### Examples 2 and 3

Light-emitting devices were manufactured in the same manner as in Example 1, except that compounds shown in Table 3 were used as the p-dopant.

### Comparative Examples 1 and 2

Light-emitting devices were manufactured in the same manner as in Example 1, except that compounds shown in Table 3 were used as the p-dopant.

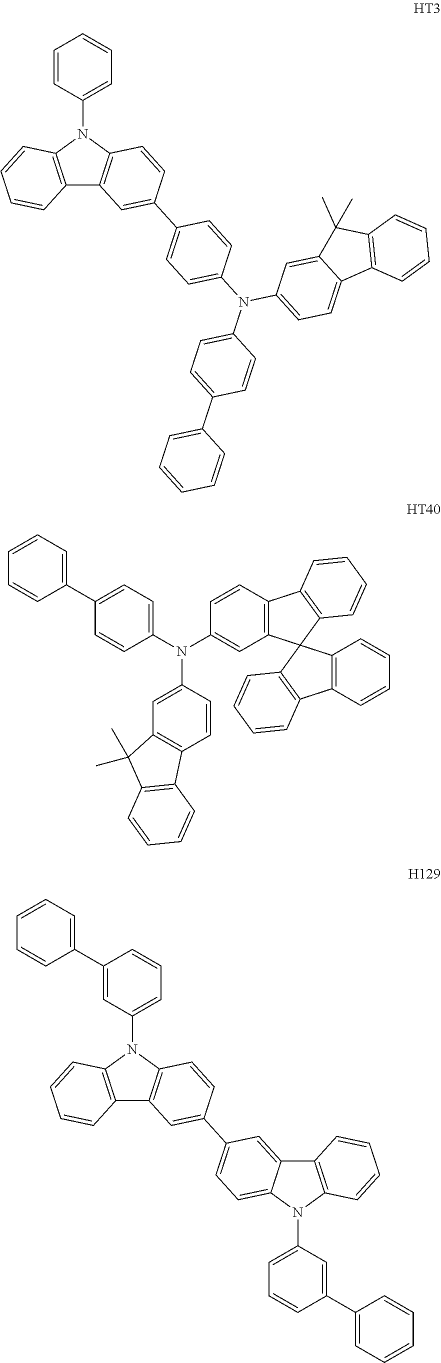 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2cccc(-n3c4ccccc4c4cc(-c5ccc6c(c5)c5ccccc5n6-c5cccc(-c6ccccc6)c5)ccc43)c2)cc1]

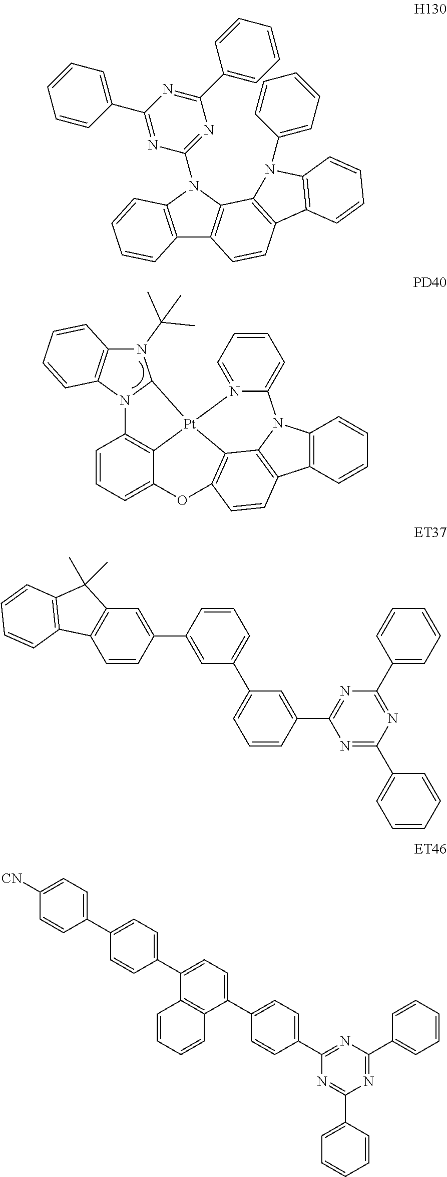

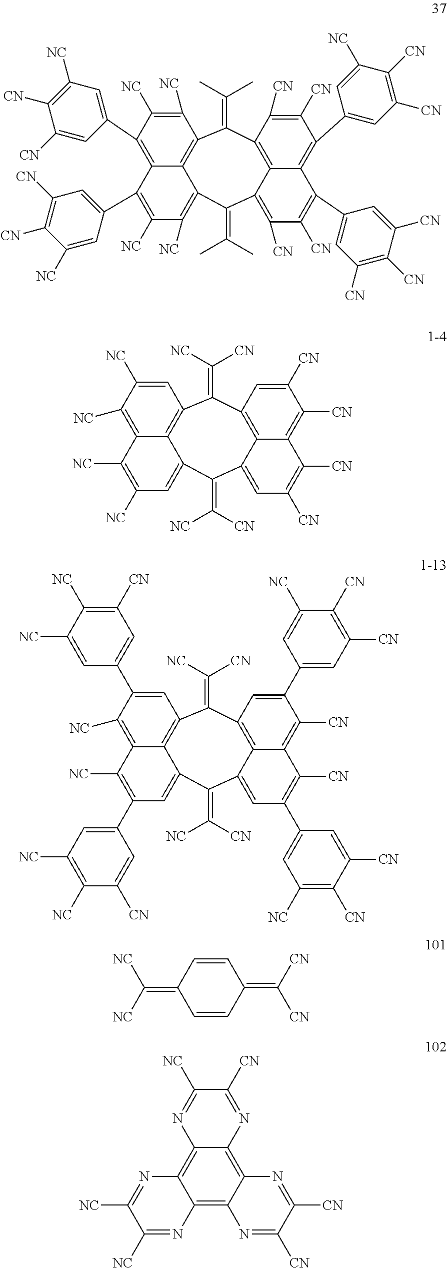

To evaluate the characteristics of the light-emitting devices, the driving voltage (V) at a luminance of 1,000 cd/m² and current efficiency were measured, and the results are shown in Table 3.

The driving voltage of the light-emitting devices was measured by using a source meter (Keithley Instrument, 2400 series), and the current efficiency of the organic light-emitting devices (candela per ampere, cd/A) was measured by using a luminance meter CS-2000 (Konica Minolta). In addition, to evaluate the device lifespan, values obtained by comparing a time taken to reach 95% of the initial luminance with a time measured in each of Examples 1 to 3 and Comparative Examples 1 and 2 were calculated. The lifespan results (%) are reported relative to Comparative Example 1.

TABLE 3

| | | Driving | Current | |
|---|---|---|---|---|
| | | voltage | efficiency | Relative |
| | Compound | (V) | (cd/A) | lifespan |
| Example 1 | 37 | 3.8 | 16.9 | 119% |
| Example 2 | 1-4 | 3.4 | 16.9 | 122% |
| Example 3 | 1-13 | 3.7 | 16.9 | 120% |
| Comparative | 101 | 5.4 | 16.7 | 100% |
| Example 1 | | | | |
| Comparative | 102 | 4.2 | 16.8 | 115% |
| Example 2 | | | | |

Referring to Table 3, it was confirmed that the light-emitting devices of Examples 1 to 3 had excellent efficiency and driving voltage, and long lifespan compared to the light-emitting devices of Comparative Examples 1 and 2.

According to the one or more embodiments, at least one compound represented by Formula 1 may be used for forming a hole injection region, and due to its suitability with a HOMO energy level of a hole transport layer and its suitable electron mobility, a light-emitting device including the compound may have improved characteristics in terms of driving voltage, efficiency, lifespan, or the like.

It should be understood that exemplary embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A light-emitting device comprising:
a first electrode;
a second electrode facing the first electrode; and
an interlayer arranged between the first electrode and the second electrode, wherein the interlayer comprises an emission layer,
wherein the interlayer further comprises at least one compound represented by Formula 1: 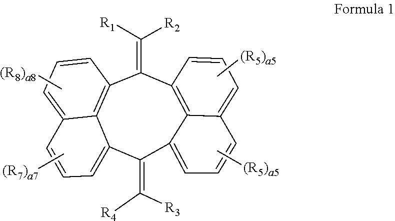
wherein, in Formula 1,
R₁ to R₈ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryl group unsubstituted or substituted with at least one R₁₀ₐ, a C₇-C₆₀ alkyl aryl group unsubstituted or substituted with at least one R₁₀ₐ, a C₇-C₆₀ aryl alkyl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₂-C₆₀ alkyl heteroaryl group unsubstituted or substituted with at least one R₁₀ₐ, a C₂-C₆₀ heteroaryl alkyl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a C₈-C₆ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)(Q₁), —S(═O)₂(Q₁), —P(Q₁)(Q₂), or —P(═O)(Q₁)(Q₂),
a5 to a8 are each independently an integer from 1 to 3, wherein, when a5 is 2 or more, each R₅ is identical to or different from the others, when a6 is 2 or more, each R₆ is identical to or different from the others, when a7 is 2 or more, each R₇ is identical to or different from the others, and when a8 is 2 or more, each R₈ is identical to or different from the others,
R₁₀ₐ is:
hydrogen, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, or a phosphoric acid group or a salt thereof;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)(Q₁₁), —S(═O)₂(Q₁₁), —P(Q₁₁)(Q₁₂), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ aryl alkyl group, or a C₂-C₆₀ heteroaryl alkyl group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)(Q₂₁), —S(═O)₂(Q₂₁), —P(Q₂₁)(Q₂₂), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)(Q₃₁), —S(═O)₂(Q₃₁), —P(Q₃₁)(Q₃₂), or —P(═O)(Q₃₁)(Q₃₂),
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group; or
a C₃-C₆₀ carbocyclic group, a C₁-C₆₀ heterocyclic group, a C₇-C₆₀ aryl alkyl group, a C₂-C₆₀ heteroaryl alkyl group, a C₆-C₆₀ aryloxy group, a C₆-C₆₀ arylthio group, a C₁-C₆₀ heteroaryloxy group, or a C₁-C₆₀ heteroarylthio group,
each unsubstituted or substituted with deuterium, —F, a cyano group, a C₁-C₆₀ alkyl group, a C₁-C₆₀ alkoxy group, a C₁-C₆₀ alkylthio group, a phenyl group, a biphenyl group, or a combination thereof, and
wherein Formula 1 comprises an electron-withdrawing group (EWG).

2. The light-emitting device of claim 1, wherein the EWG comprises —F, —Cl, a cyano group, a nitro group, a carbonyl group, or a combination thereof.

3. The light-emitting device of claim 1, wherein
the first electrode is an anode,
the second electrode is a cathode,
the interlayer further comprises a hole transport region arranged between the first electrode and the emission layer, wherein the hole transport region comprises a hole injection layer, a hole transport layer, an electron-blocking layer, an emission auxiliary layer, or a combination thereof; and/or
the interlayer further comprises an electron transport region arranged between the second electrode and the emission layer, wherein the electron transport region comprises a hole-blocking layer, an electron transport layer, an electron injection layer, or a combination thereof.

4. The light-emitting device of claim 3, wherein the hole transport region comprises the at least one compound represented by Formula 1.

5. The light-emitting device of claim 3, wherein the hole injection layer comprises the at least one compound represented by Formula 1.

6. The light-emitting device of claim 1, wherein
the emission layer comprises a first host, a second host, and a dopant, and
the dopant comprises a ligand comprising a metal and an imidazole moiety.

7. The light-emitting device of claim 6, wherein the first host is a hole-transporting host.

8. The light-emitting device of claim 6, wherein the second host is an electron-transporting host.

9. The light-emitting device of claim 6, wherein the dopant comprises at least one compound represented by Formula 401: 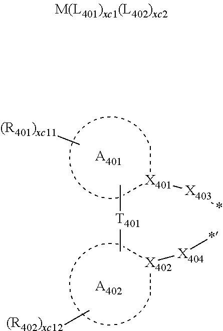
wherein, in Formulae 401 and 402,
M is titanium (Ti), cobalt (Co), copper (Cu), zinc (Zn), zirconium (Zr), ruthenium (Ru), rhodium (Rh), palladium (Pd), rhenium (Re), platinum (Pt), gold (Au), osmium (Os), iridium (Ir), or rhenium (Re),
L₄₀₁ is a ligand represented by Formula 402,
xc1 is 1, 2, or 3, and when xc1 is 2 or more, two or more of L₄₀₁ are identical to or different from each other, two of ring A₄₀₁ are optionally linked to each other via a linking group T₄₀₁, and two of ring A₄₀₂ are optionally linked to each other via a linking group T₄₀₃,
L₄₀₂ is an organic ligand,
xc2 is 0, 1, 2, 3, or 4, and when xc2 is 2 or more, two or more of L₄₀₂ are identical to or different from each other,
X₄₀₁ and X₄₀₂ are each independently nitrogen or carbon,
ring A₄₀₁ and ring A₄₀₂ are each independently a C₃-C₆₀ carbocyclic group or a C₁-C₆₀ heterocyclic group,
T₄₀₁ to T₄₀₃ are each independently a single bond, —O—, —S—, —C(═O)—, —N(Q₄₁₁)-, —C(Q₄₁₁)(Q₄₁₂)-, —C(Q₄₁₁)═C(Q₄₁₂)-, —C(Q₄₁₁)═, or ═C═,
X₄₀₃ and X₄₀₄ are each independently a chemical bond, —O—, —S—, —N(Q₄₁₃)—, —B(Q₄₁₃)-, —P(Q₄₁₃)-, —C(Q₄₁₃)(Q₄₁₄)-, or —Si(Q₄₁₃)(Q₄₁₄)-,
R₄₀₁ and R₄₀₂ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group that is unsubstituted or substituted with at least one R₁₀ₐ, a C₁-C₂₀ alkoxy group that is unsubstituted or substituted with at least one R₁₀ₐ, a C₁-C₂₀ alkylthio group unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₇-C₆₀ alkyl aryl group unsubstituted or substituted with at least one R₁₀ₐ, a C₇-C₆₀ aryl alkyl group unsubstituted or substituted with at least one R₁₀ₐ, a C₂-C₆₀ alkyl heteroaryl group unsubstituted or substituted with at least one R₁₀ₐ, a C₂-C₆₀ heteroaryl alkyl group unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, —Si(Q₄₀₁)(Q₄₀₂)(Q₄₀₃), —N(Q₄₀₁)(Q₄₀₂), —B(Q₄₂₁)(Q₄₀₂), —C(═O)(Q₄₀₁), S(═O)(Q₄₀₁), —S(═O)₂(Q₄₀₁), —P(Q₄₀₁)(Q₄₀₂), or —P(═O)(Q₄₀₁)(Q₄₀₂),
optionally, R₄₀₁ and R₄₀₂ are linked to each other to form a ring,
R₁₀ₐ is:
deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, or a phosphoric acid group or a salt thereof;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, a C₁-C₆₀ heteroaryloxy group, a C₁-C₆₀ heteroarylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)(Q₁₁), —S(═O)₂(Q₁₁), —P(Q₁₁)(Q₁₂), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, a C₁-C₆₀ heteroaryloxy group, a C₁-C₆₀ heteroarylthio group, a C₇-C₆₀ aryl alkyl group, or a C₂-C₆₀ heteroaryl alkyl group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, a C₁-C₆₀ heteroaryloxy group, a C₁-C₆₀ heteroarylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)(Q₂₁), —S(═O)₂(Q₂₁), —P(Q₂₁)(Q₂₂), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)(Q₃₁), —S(═O)₂(Q₃₁), —P(Q₃₁)(Q₃₂), or —P(═O)(Q₃₁)(Q₃₂),
Q₁₁ to Q₁₃, Q₂₁ to Q₂₃, Q₃₁ to Q₃₃, Q₄₁₁ to Q₄₁₄, and Q₄₀₁ to Q₄₀₃ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₇-C₆₀ aryl alkyl group, a C₂-C₆₀ heteroaryl alkyl group, a C₆-C₆₀ aryloxy group, a C₆-C₆₀ arylthio group, a C₁-C₆₀ heteroaryloxy group, or a C₁-C₆₀ heteroarylthio group, each unsubstituted or substituted with deuterium, —F, a cyano group, a C₁-C₆₀ alkyl group, a C₁-C₆₀ alkoxy group, a C₁-C₆₀ alkylthio group, a phenyl group, a biphenyl group, or a combination thereof;
xc11 and xc12 are each independently an integer from 0 to 10,
* and *′ in Formula 402 each indicate a binding site to M in Formula 401, and
one of ring A₄₀₁ or ring A₄₀₂ includes an imidazole moiety.

10. The light-emitting device of claim 1, wherein the at least one compound of Formula 1 is symmetrical.

11. The light-emitting device of claim 1, wherein R₁ to R₄ in Formula 1 are each independently a C₁-C₆₀ alkyl group unsubstituted or substituted with at least one R₁₀ₐ.

12. The light-emitting device of claim 1, wherein R₅ to R₈ in Formula 1 are each independently a C₆-C₆₀ aryl group unsubstituted or substituted with at least one R₁₀ₐ.

13. The light-emitting device of claim 1, wherein R₁ to R₄ in Formula 1 each comprises the EWG.

14. The light-emitting device of claim 1, wherein the at least one compound represented by Formula 1 comprises one of the following compounds:

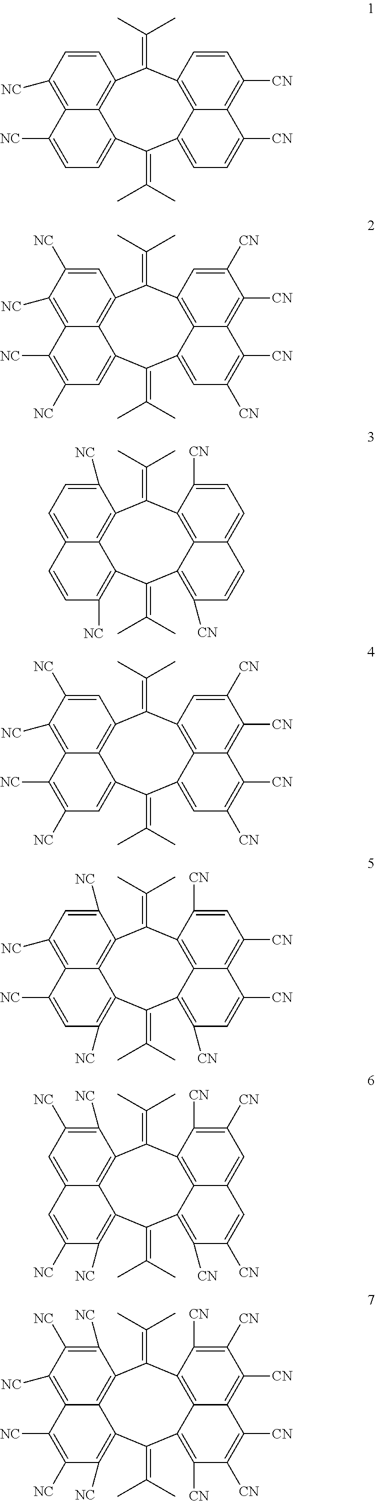

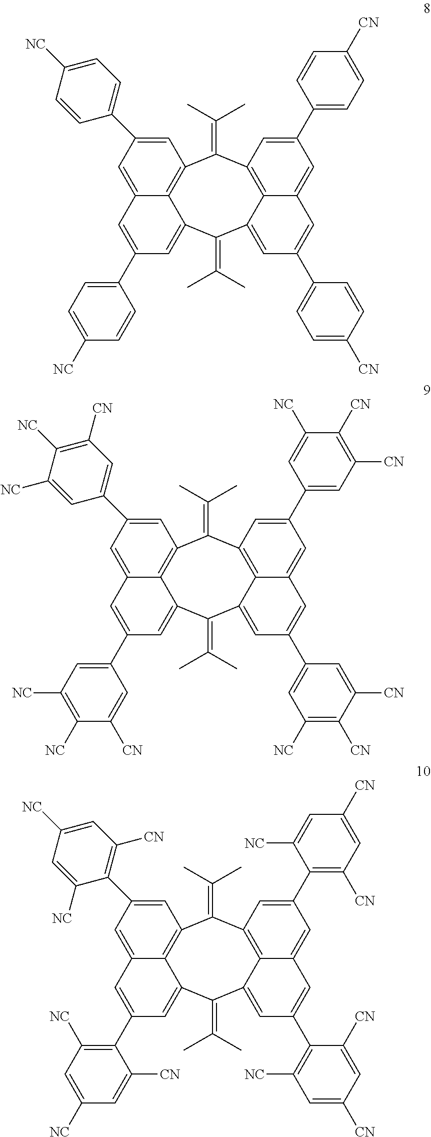

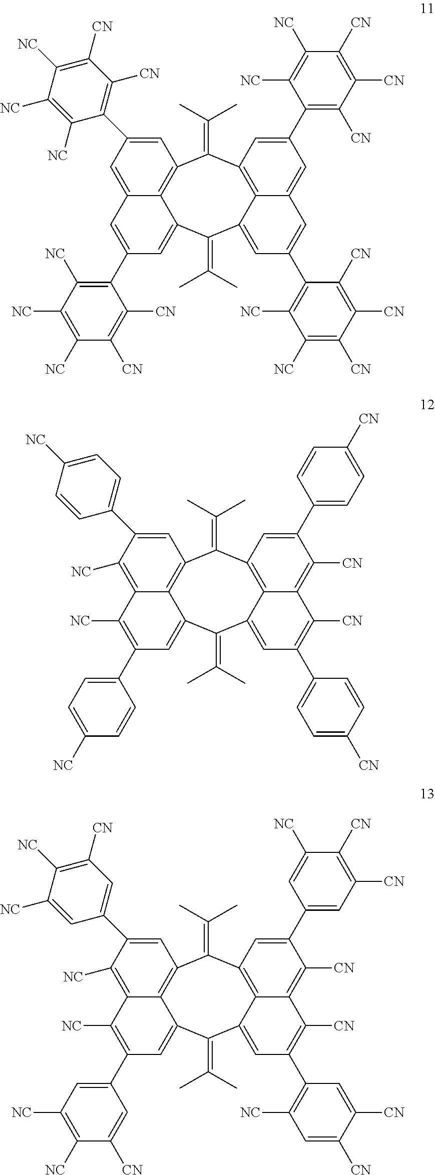 [SMILES: CC(C)=c1c2cc(-c3c(C#N)c(C#N)c(C#N)c(C#N)c3C#N)cc3cc(-c4c(C#N)c(C#N)c(C#N)c(C#N)c4C#N)cc(c(=C(C)C)c4cc(-c5c(C#N)c(C#N)c(C#N)c(C#N)c5C#N)cc5cc(-c6c(C#N)c(C#N)c(C#N)c(C#N)c6C#N)cc1c54)c32 ; CC(C)=c1c2cc(-c3ccc(C#N)cc3)c(C#N)c3c(C#N)c(-c4ccc(C#N)cc4)cc(c(=C(C)C)c4cc(-c5ccc(C#N)cc5)c(C#N)c5c(C#N)c(-c6ccc(C#N)cc6)cc1c54)c32 ; CC(C)=c1c2cc(-c3cc(C#N)c(C#N)c(C#N)c3)c(C#N)c3c(C#N)c(-c4cc(C#N)c(C#N)c(C#N)c4)cc(c(=C(C)C)c4cc(-c5cc(C#N)c(C#N)cc5C#N)c(C#N)c5c(C#N)c(-c6cc(C#N)c(C#N)c(C#N)c6)cc1c54)c32]

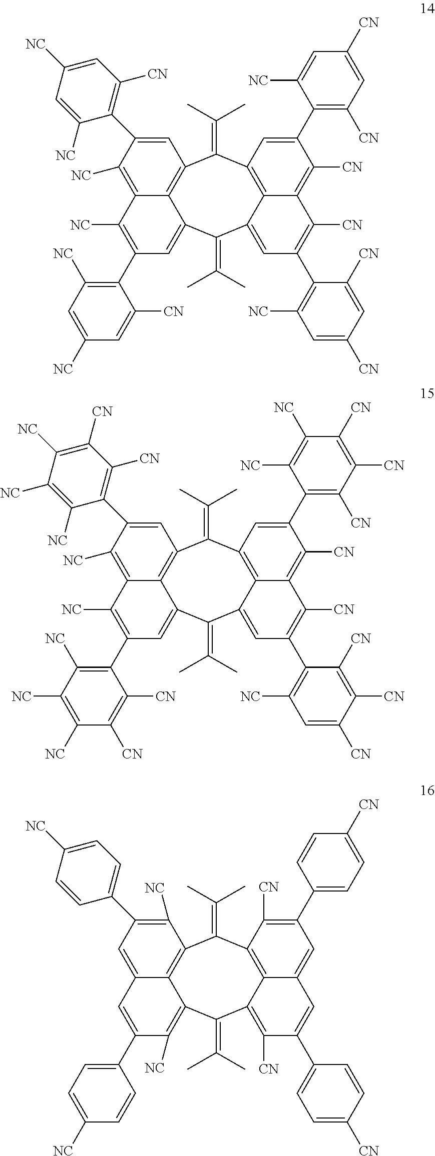

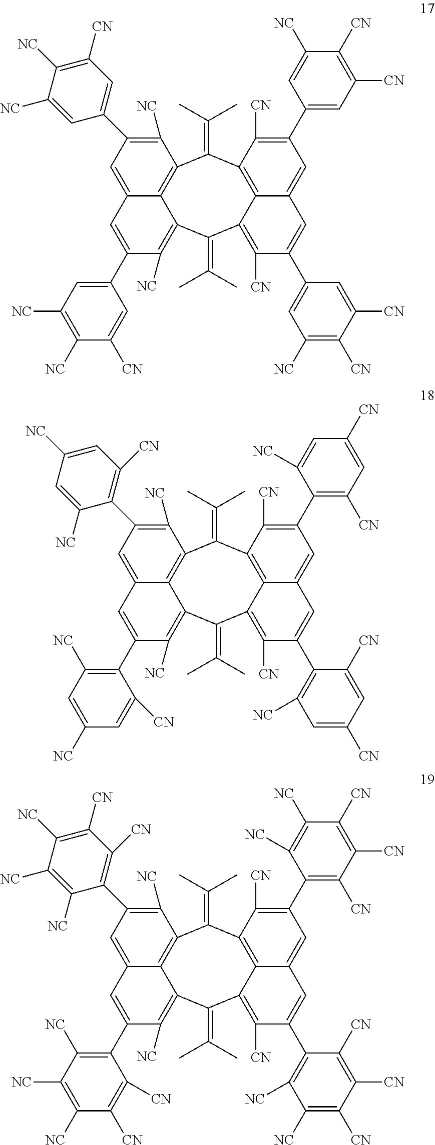

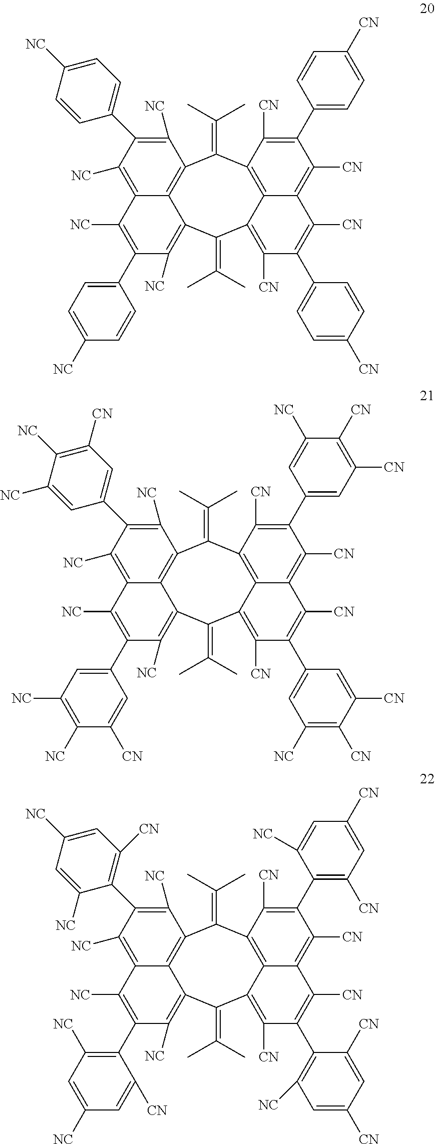

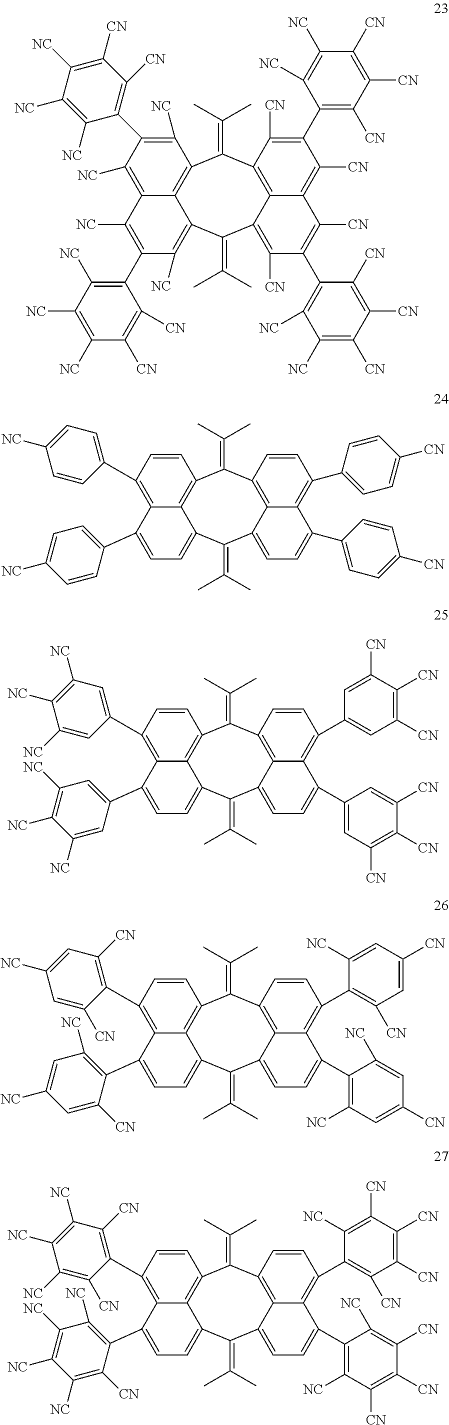

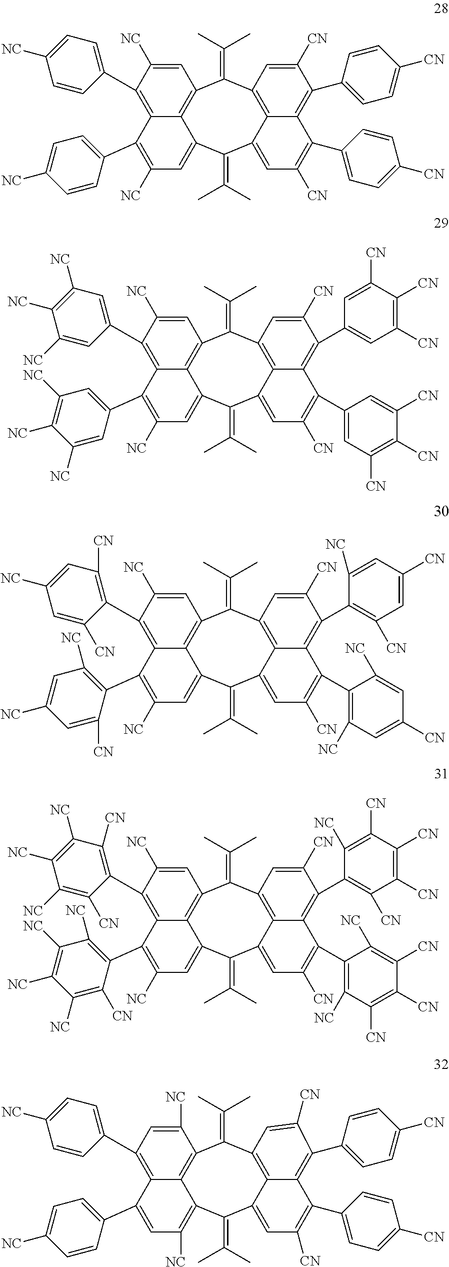

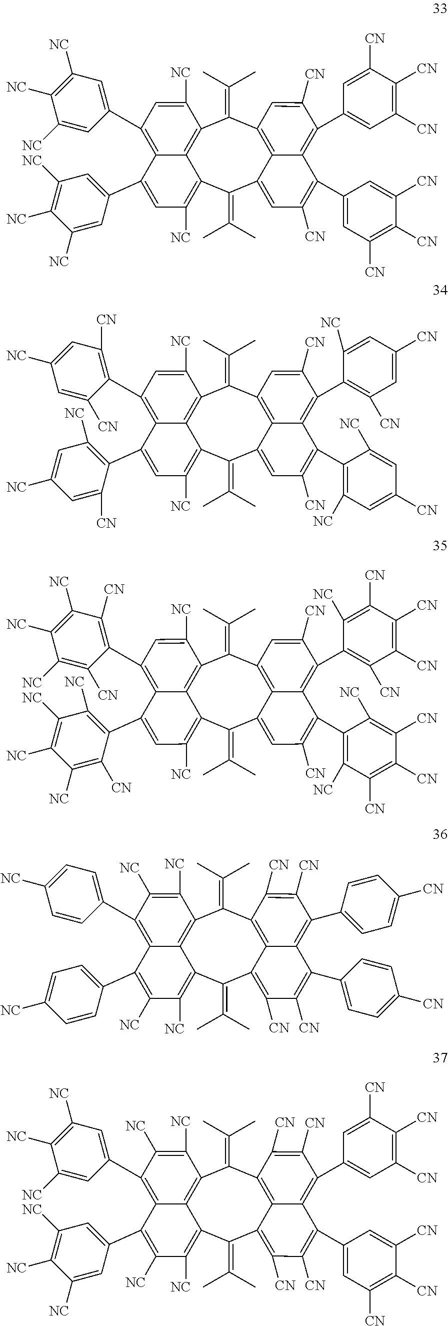

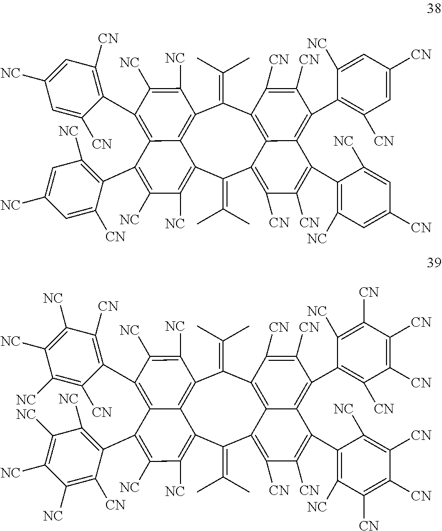 [SMILES: CC(C)=c1c2c(C#N)c(C#N)c(-c3c(C#N)cc(C#N)cc3C#N)c3c(-c4c(C#N)cc(C#N)cc4C#N)c(C#N)c(C#N)c(c(=C(C)C)c4c(C#N)c(C#N)c(-c5c(C#N)cc(C#N)cc5C#N)c5c(-c6c(C#N)cc(C#N)cc6C#N)c(C#N)c(C#N)c1c54)c32 ; CC(C)=c1c2c(C#N)c(C#N)c(-c3c(C#N)c(C#N)c(C#N)c(C#N)c3C#N)c3c(-c4c(C#N)c(C#N)c(C#N)c(C#N)c4C#N)c(C#N)c(C#N)c(c(=C(C)C)c4c(C#N)c(C#N)c(-c5c(C#N)c(C#N)c(C#N)c(C#N)c5C#N)c5c(-c6c(C#N)c(C#N)c(C#N)c(C#N)c6C#N)c(C#N)c(C#N)c1c54)c32]

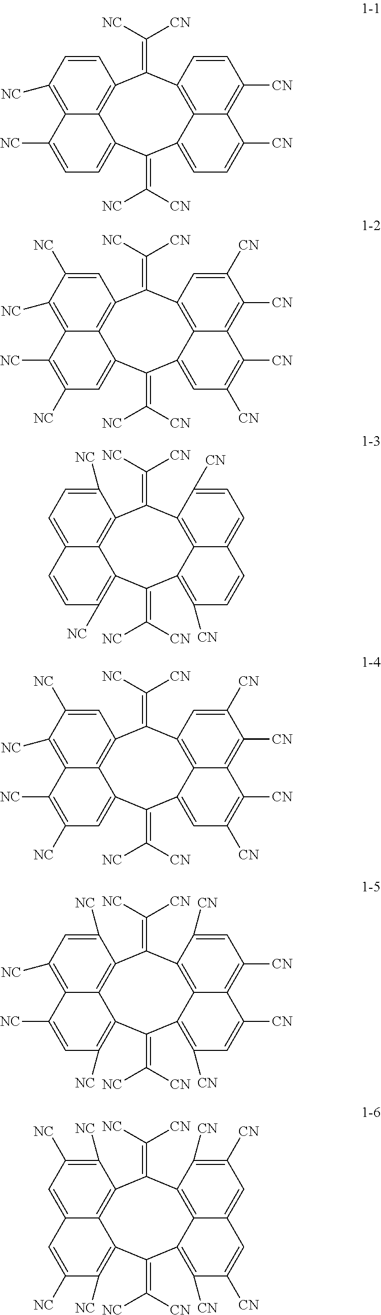

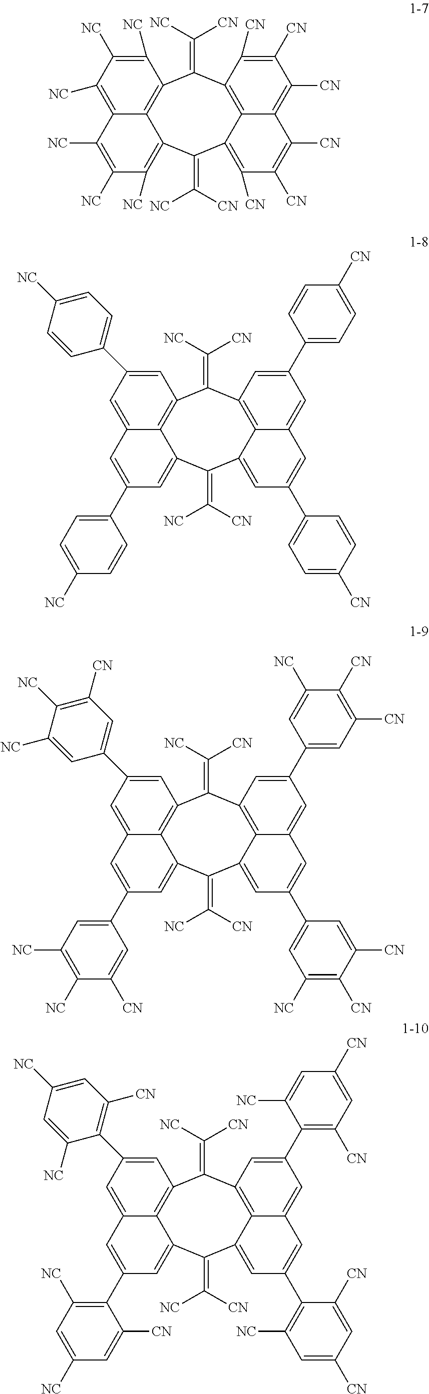

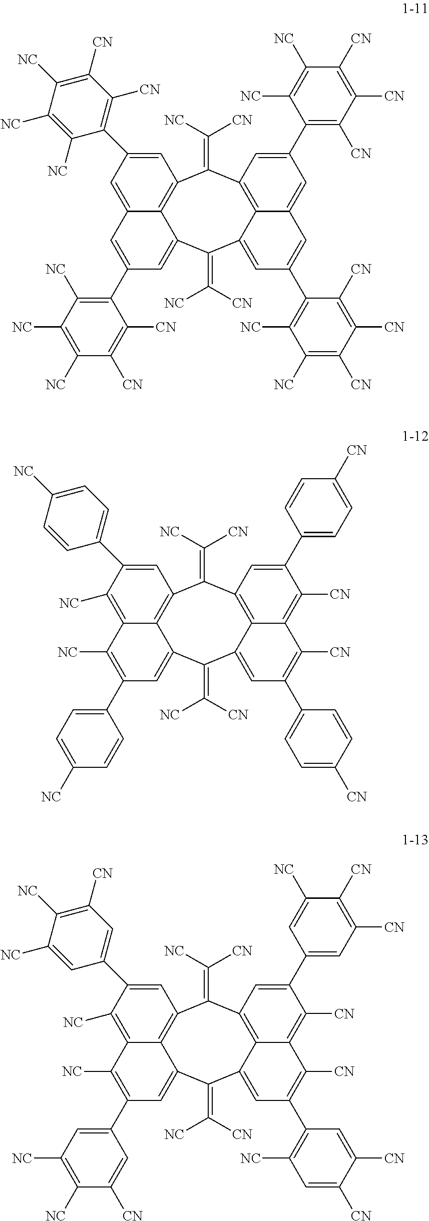

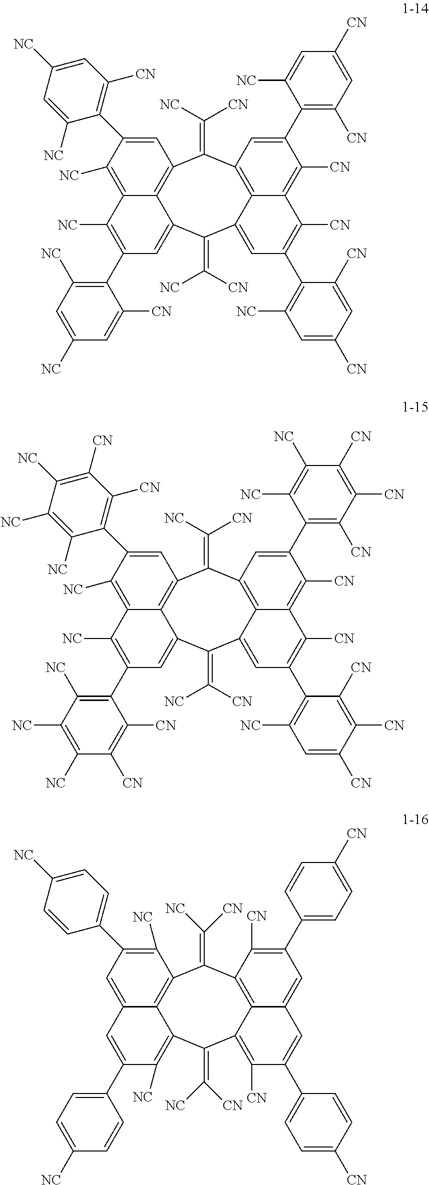

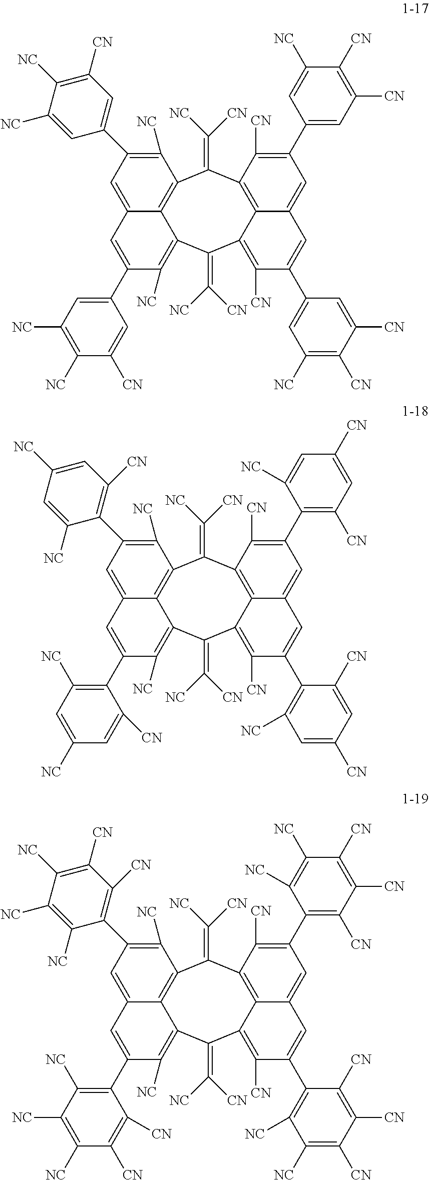

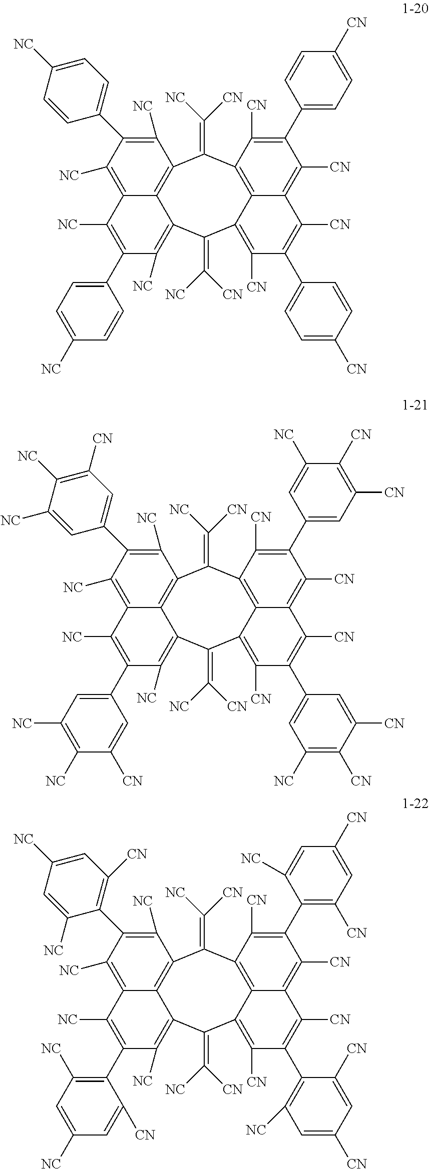

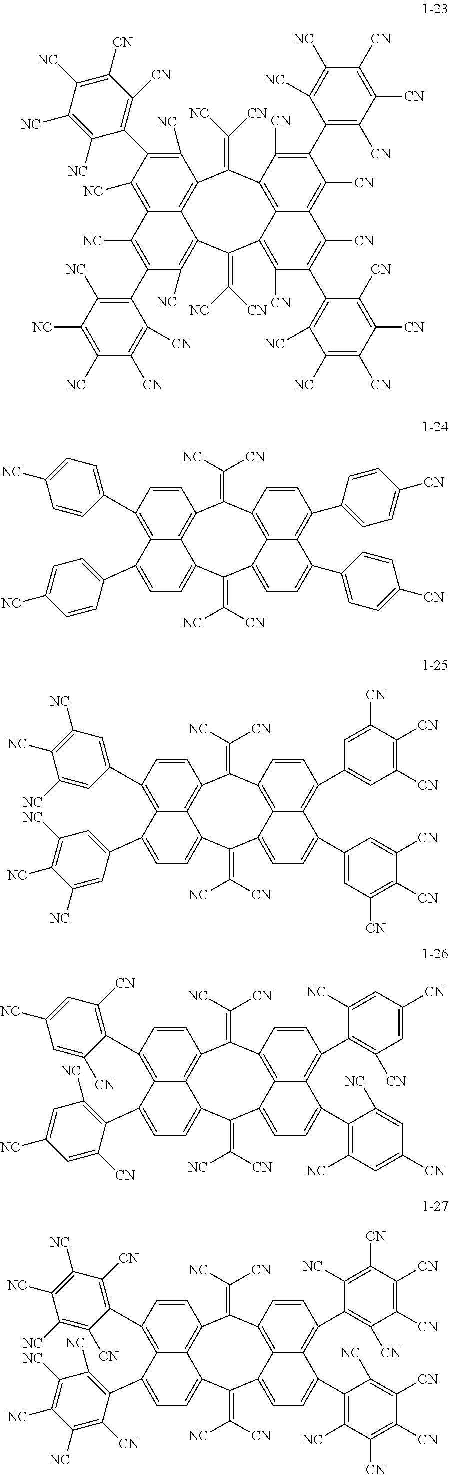

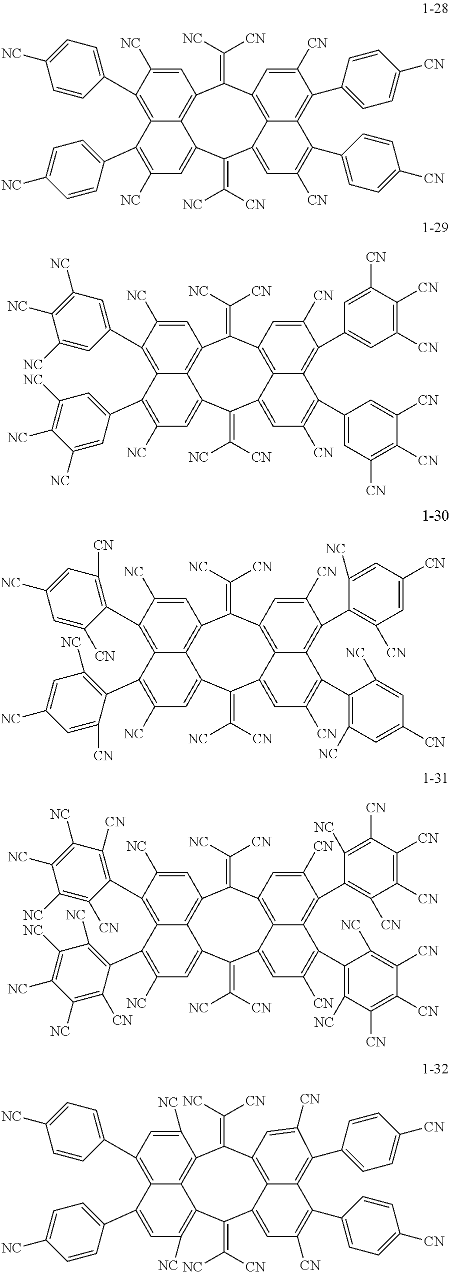

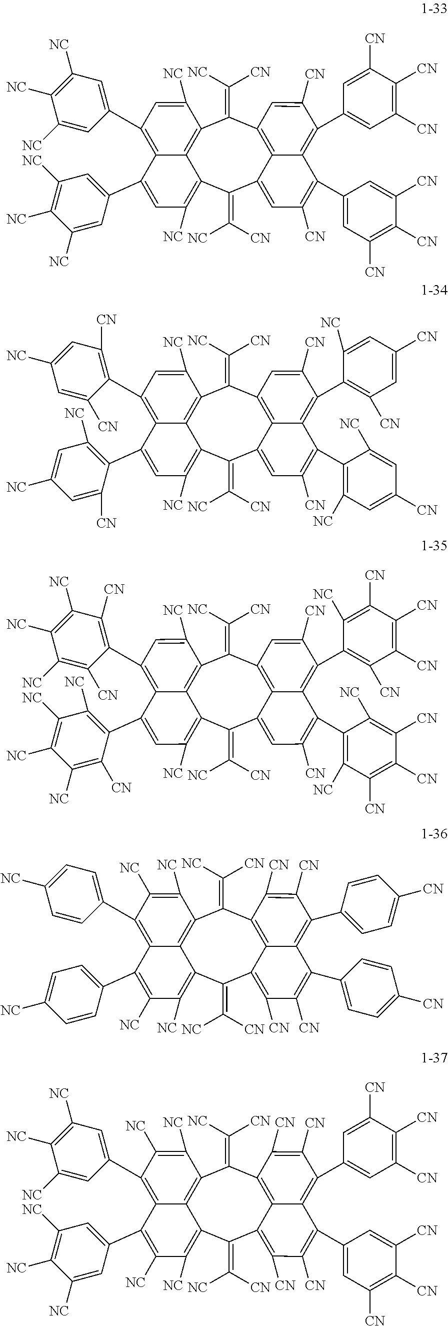

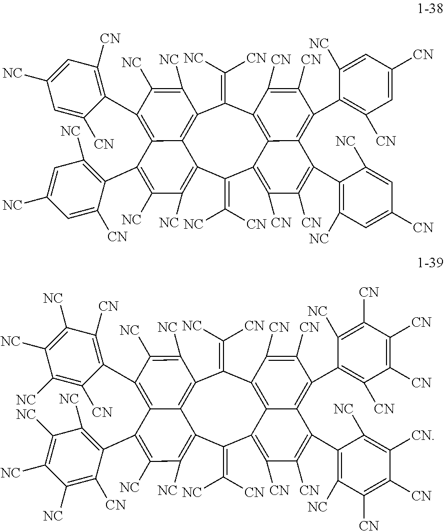 [SMILES: N#CC(C#N)=c1c2c(C#N)c(C#N)c(-c3c(C#N)cc(C#N)cc3C#N)c3c(-c4c(C#N)cc(C#N)cc4C#N)c(C#N)c(C#N)c(c(=C(C#N)C#N)c4c(C#N)c(C#N)c(-c5c(C#N)cc(C#N)cc5C#N)c5c(-c6c(C#N)cc(C#N)cc6C#N)c(C#N)c(C#N)c1c54)c32 ; N#CC(C#N)=c1c2c(C#N)c(C#N)c(-c3c(C#N)c(C#N)c(C#N)c(C#N)c3C#N)c3c(-c4c(C#N)c(C#N)c(C#N)c(C#N)c4C#N)c(C#N)c(C#N)c(c(=C(C#N)C#N)c4c(C#N)c(C#N)c(-c5c(C#N)c(C#N)c(C#N)c(C#N)c5C#N)c5c(-c6c(C#N)c(C#N)c(C#N)c(C#N)c6C#N)c(C#N)c(C#N)c1c54)c32]

15. An electronic apparatus comprising the light-emitting device of claim 1.

16. The electronic apparatus of claim 15, further comprising:
a thin-film transistor,
wherein the thin-film transistor comprises a source electrode and a drain electrode, and
the first electrode of the light-emitting device is electrically connected to at least one of the source electrode or the drain electrode.

17. The electronic apparatus of claim 15, further comprising a display module, a processor, a memory, and a power module.

18. The electronic apparatus of claim 15, wherein the electronic apparatus comprises an electronic apparatus for displaying images, a wearable electronic apparatus, or an electronic apparatus for a vehicle.

19. The electronic apparatus of claim 15, wherein the electronic apparatus comprises one of a smartphone, a tablet PC, a laptop, a TV, a desk monitor, smart glasses, a head-mounted display, a smart watch, a vehicle dashboard, a center fascia, a center information display (CID) on a vehicle dashboard, or a room mirror display.

20. A compound represented by Formula 1: 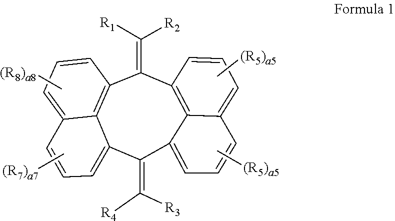
wherein, in Formula 1,
R₁ to R₈ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryl group unsubstituted or substituted with at least one R₁₀ₐ, a C₇-C₆₀ alkyl aryl group unsubstituted or substituted with at least one R₁₀ₐ, a C₇-C₆₀ aryl alkyl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₂-C₆₀ alkyl heteroaryl group unsubstituted or substituted with at least one R₁₀ₐ, a C₂-C₆₀ heteroaryl alkyl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a C₈-C₆ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)(Q₁), —S(═O)₂(Q₁), —P(Q₁)(Q₂), or —P(═O)(Q₁)(Q₂),
a5 to a8 are each independently an integer from 1 to 3, wherein, when a5 is 2 or more, each R₅ is identical to or different from the others, when a6 is 2 or more, each R₆ is identical to or different from the others, when a7 is 2 or more, each R₇ is identical to or different from the others, and when a8 is 2 or more, each R₈ is identical to or different from the others,
R₁₀ₐ is:
hydrogen, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, or a phosphoric acid group or a salt thereof;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₆₀ carbocyclic group, a C₁-C₆₀ heterocyclic group, a C₁-C₆₀ aryloxy group, a C₆-C₆₀ arylthio group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O)(Q₁₁), —S(═O)(Q₁₁), —S(═O)₂(Q₁₁), —P(Q₁₁)(Q₁₂), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, or a C₁-C₆₀ heteroarylthio group, each unsubstituted or substituted with deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O)(Q₂₁), —S(═O)(Q₂₁), —S(═O)₂(Q₂₁), —P(Q₂₁)(Q₂₂), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)(Q₃₁), —S(═O)₂(Q₃₁), —P(Q₃₁)(Q₃₂), or —P(═O)(Q₃₁)(Q₃₂),
Q₁ to Q₃, Q₁₁ to Q₁₃, Q₂₁ to Q₂₃, and Q₃₁ to Q₃₃ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group; or
a C₃-C₆₀ carbocyclic group, a C₁-C₆₀ heterocyclic group, a C₇-C₆₀ aryl alkyl group, a C₂-C₆₀ heteroarylalkyl group, a C₆-C₆₀ aryloxy group, a C₆-C₆₀ arylthio group, a C₁-C₆₀ heteroaryloxy group, or a C₁-C₆₀ heteroarylthio group,
each unsubstituted or substituted with deuterium, —F, a cyano group, a C₁-C₆₀ alkyl group, a C₁-C₆₀ alkoxy group, a C₁-C₆₀ alkylthio group, a phenyl group, a biphenyl group, or a combination thereof, and
Formula 1 comprises an electron-withdrawing group (EWG).
